SEQUENCE LISTING (60 sequences)

SEQ ID NO: 1 - protein, 10 aa, Human immunodeficiency virus
HIGPGRAFYT

SEQ ID NO: 2 - protein, 10 aa, Human immunodeficiency virus
SIGPGRAFGC

SEQ ID NO: 3 - protein, 4 aa, Human immunodeficiency virus
GPGR

SEQ ID NO: 4 - protein, 18 aa, Human immunodeficiency virus
RKSIRIQRGPGRAFVTIG

SEQ ID NO: 5 - protein, 6 aa, Human immunodeficiency virus
GPGRAF

SEQ ID NO: 6 - protein, 9 aa, Human immunodeficiency virus
CKRIHIGPG

SEQ ID NO: 7 - protein, 11 aa, Human immunodeficiency virus
IHIGPGRAFYT

SEQ ID NO: 8 - protein, 10 aa, Human immunodeficiency virus
KRIHIGPGRA

SEQ ID NO: 9 - protein, 5 aa, Human immunodeficiency virus
KRIHI

SEQ ID NO: 10 - protein, 14 aa, Human immunodeficiency virus
AVVFQTKRSKQVCA

SEQ ID NO: 11 - protein, 4 aa, Human immunodeficiency virus
GRAF

SEQ ID NO: 12 - protein, 4 aa, Human immunodeficiency virus
RAFY

SEQ ID NO: 13 - protein, 23 aa, Human immunodeficiency virus
YNKRKRIHIGPGRAFYTTKNIIG

SEQ ID NO: 14 - protein, 18 aa, Human immunodeficiency virus
KSIRIQRGPGRAFVTIGK

SEQ ID NO: 15 - protein, 5 aa, Human immunodeficiency virus
KSIRI

SEQ ID NO: 16 - protein, 5 aa, Human immunodeficiency virus
FVTIG

SEQ ID NO: 17 - protein, 14 aa, Human immunodeficiency virus
KSIHIGPGRAFYTT

SEQ ID NO: 18 - protein, 18 aa, Artificial Sequence
  Synthetic Peptide
CRKSIHCGPGRCFYTTGC

SEQ ID NO: 19 - protein, 18 aa, Artificial Sequence
  Synthetic Peptide
GCKSICIGPGRACYTTCG

SEQ ID NO: 20 - protein, 14 aa, Human immunodeficiency virus
KRIHIGPGRAFYTT

SEQ ID NO: 21 - protein, 12 aa, Homo sapiens
LLYSQNKPKAAE

SEQ ID NO: 22 - protein, 14 aa, Homo sapiens
GVIFLTKRSRQVCA

SEQ ID NO: 23 - protein, 14 aa, Homo sapiens
AVVFVTRKNRQVCA

SEQ ID NO: 24 - protein, 16 aa, Human immunodeficiency virus
KSIRIQRGPGRAFVTI

SEQ ID NO: 25 - protein, 14 aa, Homo sapiens
IVARLKNNNRQVCI

SEQ ID NO: 26 - protein, 8 aa, Human immunodeficiency virus
KRIHIGPG

SEQ ID NO: 27 - protein, 11 aa, Human immunodeficiency virus
RIHIGPGRAFY

SEQ ID NO: 28 - protein, 17 aa, Human immunodeficiency virus
KSIRIQRGPGRAFVTIG

SEQ ID NO: 29 - protein, 12 aa, Artificial Sequence
  Synthetic Peptide
IXXGPGXXXYXT

SEQ ID NO: 30 - protein, 18 aa, Artificial Sequence
  Synthetic Peptide
CRKSIHIGPGRAFYTTGC

SEQ ID NO: 31 - protein, 15 aa, Human immunodeficiency virus
KRIHIGPGRAFYTTV

SEQ ID NO: 32 - protein, 13 aa, Human immunodeficiency virus
KRIHIGPGRAFYT

SEQ ID NO: 33 - protein, 10 aa, Human immunodeficiency virus
IHIGPGRAFY

SEQ ID NO: 34 - protein, 15 aa, Artificial Sequence
  Synthetic Peptide
XXXXXGPGXAFYTTV

SEQ ID NO: 35 - protein, 7 aa, Artificial Sequence
  Synthetic Peptide
XXXXXXX

SEQ ID NO: 36 - protein, 6 aa, Artificial Sequence
  Synthetic Peptide
XXXXXX

SEQ ID NO: 37 - protein, 20 aa, Human immunodeficiency virus
TRKSIRIQRGPGRAFVTIGK

SEQ ID NO: 38 - protein, 8 aa, Artificial Sequence
  Synthetic Peptide
XXXXXXXX

SEQ ID NO: 39 - protein, 10 aa, Human immunodeficiency virus
IHIGPGRAFY

SEQ ID NO: 40 - protein, 16 aa, Artificial Sequence
  Synthetic Peptide
CKRIHIGPGRAFYTTC

SEQ ID NO: 41 - protein, 10 aa, Human immunodeficiency virus
KRIHIGPGRA

SEQ ID NO: 42 - protein, 5 aa, Human immunodeficiency virus
IRIHI

SEQ ID NO: 43 - protein, 4 aa, Human immunodeficiency virus
FVTI

SEQ ID NO: 44 - protein, 4 aa, Human immunodeficiency virus
FYTT

SEQ ID NO: 45 - protein, 18 aa, Artificial Sequence
  Synthetic Peptide
TRCSIHCGPGRACYTTCE

SEQ ID NO: 46 - protein, 17 aa, Artificial Sequence
  Synthetic Peptide
TRCSIHIGPGRAFYTTC

SEQ ID NO: 47 - protein, 8 aa, Human immunodeficiency virus
IHIGPGRA

SEQ ID NO: 48 - protein, 8 aa, Artificial Sequence
  Synthetic Peptide
XIXXGRXX

SEQ ID NO: 49 - protein, 30 aa, Homo sapiens
DCYCRIPACIAGERRYGTCIYQGRLWAFCC

SEQ ID NO: 50 - protein, 26 aa, Helicobacter pylori
ITNALIVDYTGIYKADIGIKDGKIAG

SEQ ID NO: 51 - protein, 10 aa, Human immunodeficiency virus
IRIQRGPGRA

SEQ ID NO: 52 - protein, 8 aa, Artificial Sequence
  Synthetic Peptide
XXIXXGXX

SEQ ID NO: 53 - protein, 34 aa, Homo sapiens
DGTLGFVRGDQYAVALALIENGKVLLGVLGCPNY

SEQ ID NO: 54 - protein, 6 aa, Human immunodeficiency virus
HIGPGR

SEQ ID NO: 55 - protein, 61 aa, Glycine max
  MISC_FEATURE (12)..(12): Xaa can be Thr or His
YSKPCCDLCMCXXXXXXQCSCEDRINSCHSDCKSCMCTRSQPGQCRCLDTNDFCYKPCKSR

SEQ ID NO: 56 - protein, 61 aa, Glycine max
YSKPCCDLCMCTRSMPPQCSCEDRINSCHSDCKSCMCTRSQPGQCRCLDTNDFCYKPCKSR

SEQ ID NO: 57 - protein, 61 aa, Glycine max
YSKPCCDLCMCHIGPGRQCSCEDRINSCHSDCKSCMCTRSQPGQCRCLDTNDFCYKPCKSR

SEQ ID NO: 58 - protein, 5 aa, Human immunodeficiency virus
GPGRA

SEQ ID NO: 59 - protein, 20 aa, Human immunodeficiency virus
NNTRKSIHIGPGRAFYTTGE

SEQ ID NO: 60 - protein, 8 aa, Artificial Sequence
  Synthetic peptide
XXXXXXXX

(19) United States
(12) Patent Application Publication
(10) Pub. No.: US 2008/0206264 A1
(43) Pub. Date: Aug. 28, 2008

(54) Constrained Hiv V3 Loop Peptides as Novel Immunogens and Receptor Antagonists

(75) Inventors: Jacob Anglister, Rehovot (IL); Michal Sharon, Nes Ziona (IL); Matthieu Schapira, Lyon (FR); Susan Zolla-Pazner, New York, NY (US); Osnat Rosen, Yavne (IL)

Correspondence Address: MCKENNA LONG & ALDRIDGE LLP, 1900 K STREET, NW, WASHINGTON, DC, 20006, US

(73) Assignee: New York University, New York, NY (US)

(21) Appl. No.: 10/544,399

(22) PCT Filed: Feb. 4, 2004
(86) PCT No.: PCT/US04/03304

§ 371 Date: Feb. 13, 2006

Related U.S. Application Data
(60) Provisional application No. 60/444,682, filed on Feb. 4, 2003.

(51) Int. Cl. A61K 39/00; C07K 2/00; C07K 16/46; A61P 31/18; G06F 17/50; C12N 5/06; G01N 33/68

(52) U.S. Cl. 424/184.1; 530/300; 530/317; 530/391.7; 436/86; 435/375; 703/1; 702/19

(57) ABSTRACT

The present invention provides constrained peptides and other organic molecules, that mimic the three dimensional characteristics of the HIV-1 V3 loop peptide when bound by a highly potent human neutralizing monoclonal antibody specific for a V3 conformational epitope, which structure is determined by NMR. Methods for screening for, and designing such molecules are disclosed. These molecules are useful as immunogens for inducing broadly-neutralizing antibodies against HIV-1 as well as antagonists for inhibiting the binding of HIV-1 to the relevant co-receptors, and may therefore be used in method of preventing or treating HIV-1 infection and disease.

### BACKGROUND OF THE INVENTION

1. Field of the Invention

The present invention in the fields of structural chemistry, immunology and medicine relates to novel molecules including constrained peptides and other organic molecules, that mimic the three dimensional (3D) characteristics of the HIV-1 V3 loop peptide when bound by a highly potent human neutralizing monoclonal antibody (mAb) specific for a V3 conformational epitope. These novel molecules are useful as immunogens for inducing neutralizing antibodies to HIV-1 as well as antagonists for inhibiting the binding of HIV-1 to the relevant co-receptors.

2. Description of the Background Art

The binding of the human immunodeficiency-virus type-1 (HIV-1) to its target cells is mediated primarily by the envelope glycoprotein (gp120) of the virus. Binding of gp120 to CD4, a molecule found on the surface of both T cells and macrophages triggers conformational changes in gp120 that expose a binding site for either the CCR5 (“R5”) or the CXCR4 (“X4”) chemokine receptor. Only after binding to chemokine receptors can the virus penetrate into the target cell. The third hypervariable region of gp120 (V3 loop, residues 303-340) is directly involved in the binding to the chemokine receptors (Trkola, A et al. (1996) Nature 384:184-187; Wu, L et al. (1996) Nature 384:179-183). The V3 loop (also referred to as “V3”) sequence determines whether a virus (a) binds to the R5 co-receptor (and is designated as an “R5 virus”) and therefore infects macrophages, or (b) binds to X4 co-receptor (and is designated as an “X4 virus”) and infects T cells (Moore, J P et al. (1997) Curr. Opin. Immunol. 9:551-562 and references therein). A single mutation in the V3 loop, D329R (Asp to Arg at position 329), converted an R5 virus into an X4 virus. A double mutation of S313R and either D329Q or D333N caused the same phenotypic conversion (De Jong, J J et al. (1992) J. Virol. 66:6777-6780). Amino acid position numbering used throughout this document, with the exception of Example VIII, is based on the sequence of the HIV BH10 isolate (Ratner, L. et al. (1985) Nature 313, 277-284). Thus, small changes in the V3 sequence are sufficient to switch the virus's receptor selectivity.

Many HIV-1-neutralizing antibodies in infected individuals or in immunized animals are directed against the V3 loop, which was accordingly designated the principal neutralizing determinant of HIV-1 (Rusche, J R et al. (1988) Proc. Natl. Acad. Sci. USA 85: 3198-3202).

HIV-neutralizing antibodies against V3 are thought to prevent the binding of gp120 to either R5 or X4, thus abolishing fusion of the virus with its target cell.

Kwong and his colleagues have solved the structure of the gp120 core of both an X4 laboratory-adapted virus and of an R5 primary isolate in complex with a CD4 fragment and the Fab fragment of a gp120-specific antibody (Kwong, P D et al. (2000) J Virol 74:1961-1972; Kwong et al. (1998) Nature 393:648-659). However, crystals could be obtained only for gp120 lacking the first three variable loops, V1/V2 and V3, and the structure of V4 and V5 has not been defined. Despite the dramatic antigenic differences between the laboratory adapted X4 virus and the primary R5 isolates, the structures of their gp120 core is very similar (Kwong et al., 2000, supra). These findings, together with results of chimeric substitution and sequence analysis, led to a conclusion that the virus's choice of co-receptor and its neutralization resistance are determined by the major variable loops, V1/V2 and V3.

As an alternative to studying the V3 conformation in the context of the intact gp120 molecule, some have studied complexes of V3 peptides with antibodies that were elicited against peptides of gp120 V3 loop. Profy and Wilson, WO 94/118232 (1994) disclosed methods based on X-ray crystallography for identifying molecules which will act as antigens capable of eliciting broadly neutralizing anti-MV antibodies, and methods for producing recombinant, broadly neutralizing anti-HIV antibodies. WO 94/118232 describes the molecular structure assumed by (a) the antigenic peptide HIGPGRAFYT (termed RP142) [SEQ ID NO:1] when bound to the Fab fragment of mAb 59.1, a broadly neutralizing anti-V3 antibody, and (b) a cyclic peptide “AS” (cyclized peptide of the sequence SIGPGRAFGC [SEQ ID NO:2] which is shown below with its organic linker chain)

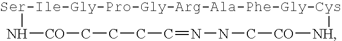

when it was bound to the Fab of antibody 58.2, a second broadly neutralizing antibody. Other publications from Wilson's group described the crystal structures of V3_{MN} peptides bound to three murine mAbs generated against a cyclic 40-residue V3 peptide comprising the entire V3 loop (Ghiara, J B et al. (1994) Science 264, 82-85; Rini, J M et al. (1993) Proc. Natl. Acad. Sci. USA 90, 6325-6329; Stanfield et al. (1999) Struct. Fold. Des. 7, 131-142). An extended conformation and multiple turn conformations were observed, respectively, for the N- and C-terminal segments of the V3 loop flanking the central GPGR [SEQ ID NO:3] sequence. The GPGR segment itself was found to adopt dual conformations. However, the short epitopes recognized by these antibodies which had been induced using synthetic peptides did not permit the determination of the global conformation of the V3_{MN} loop.

Nuclear magnetic resonance (NMR) spectroscopy is a powerful tool for protein structure analysis. In the absence of diffraction-quality crystals, NMR offers the most precise method available for determining protein structure, and provides information on the nature of protein structure most relevant to a solution state. Multi-dimensional NMR techniques have been successfully applied to proteins with molecular weight up to about 50 kDa, using a variety of pulse sequences (Kelly et al., Proc. Natl. Acad. Sci. USA 98:13025 (2001); Garrett et al., Nature Struct. Biol. 6:166 (1999). Techniques which have been exploited extensively to determine details about protein structure utilize nuclear Overhauser enhancement effects which can provide information about interatomic distances and through-bond coupling parameters which can reveal dihedral angles between coupled atoms. Vicinal spin-spin coupling constants ³J_{HNHα} provide a reliable basis for confirming secondary structures suggested by interproton distance maps (K. Wüthrich, NMR of Proteins and Nucleic Acids, Wiley-Interscience, New York, Chap. 9, 1986).

While a number of NMR studies of V3 peptides in solution have been carried out, only one reported analysis of a V3 peptide bound to an antibody fragment (the Fv fragment). A strain-specific HIV-1-neutralizing murine mAb named “0.5β” raised against the gp120_{IIIB} protein recognizes a significantly longer epitope in a V3_{IIIB} peptide (RKSIRIQRGPGRAFVTIG [SEQ ID NO:4]) than that recognized by the anti-peptide antibodies noted above. The peptide bound to this antibody formed a β-hairpin with an irregular turn around GPGR (Tugarinov, V et al. (1999) Nature Struct. Biol. 6:331-335) that was not observed in the X-ray studies noted above. The HIV-1_{IIIB} strain includes a two residue insertion, QR, near the tip of the V3 loop; this “minority” sequence is found in less than 10% of HIV-1 isolates. Moreover, this insertion is not found in the HIV-1 MN strain (HIV_{MN} or just “MN”), which is representative of subtype B viruses common in Europe and North America (Myers, G et al. (1996 or updates) Human retroviruses and AIDS: a compilation and analysis of nucleic acid and amino sequences (Los Alamos National Lab database found at the Worldwide web site with the URL “hiv.lanl.gov”). Indeed, the present invention is based in part on a study of a V3 peptide derived from the MN strain bound to the 447-52D mAb. It is noteworthy that extensive NMR studies of isolated V3 peptides do not indicate any stable structure in solution, although transient turns were found around the GPGR region (Catasti, P et al. 1996, J Biol Chem 271:8236; Catasti, P. et al. (1995) J. Biol. Chem. 270, 2224-2232; Chandrasekhar, K et al. (1991) Biochemistry 30:9187-9194; de Lorimier, R et al. (1994) Biochemistry 33, 2055-2062; Dettin, M et al. (1993) Biochem. Biophys. Res. Commun. 191, 364-370; Dettin, M. et al. (1997) J. Pept. Sci. 3, 15-30; Ghiara, J. B. et al. (1997) J. Mol. Biol. 266, 31-39; Gupta, G. et al. (1993) J. Biomol. Struct. Dyn. 11, 345-366; Huang, X et al. (1997). Biochemistry 36, 10846-10856; Huang, X. et al. (1996). FEBS Lett. 393, 280-286; Markert, R L et al. (1996) Eur. J. Biochem. 237, 188-204; Sarma, A V et al. (1997) Biochem. Biophys. Meth. 34, 83-98; Vranken, W F et al. (1996) Eur. J. Biochem. 236, 100-108; Vu, H M et al. (1996) Biochemistry 35, 5158-5165; Vu, H M et al. (1999) J. Virol. 73, 746-750; Zvi, A et al. (1992) Biochemistry 31, 6972-6979). Addition of 20% TFE usually stabilizes an α-helical conformation immediately C-terminal to GPGR (Catasti et al., supra; Chandrasekhar et al., supra; Vranken et al., supra; Zvi et al., supra). NMR studies on peptides modified by cyclization (Cabezas, E et al. (2000) Biochemistry 39:14377-14391; Chandrasekhar et al., supra; Gupta et al., supra; Huisman, J G et al. (2000) Biochemistry 39, 10866-10876; Tolman, R L et al. (1993) Int. J. Pept. Protein Res. 41, 455-466; Vranken et al., supra; Vranken, W F et al., (1995) FEBS Lett. 374, 117-121; Vranken, W F (2001) Eur. J. Biochem. 268, 2620-2628) by replacement of an Ala residue (Ala316) with the conformationally-restricted residue α-aminoisobutyric acid (Cabezas et al., supra; Ghiara et al., supra) by glycosylation (Huang et al., supra; Markert et al., supra) through attachment to resin beads (Jelinek, R et al. (1997) J. Magn. Reson. 125, 185-187) through attachment to a bacteriophage viral coat protein (Jelinek, R. et al. (1997) J. Mol. Biol. 266, 649-655) and through attachment to carrier proteins, such as BPTI (Wu, G et al. (2000) J. Biol. Chem. 275, 36645-36652) and MUC1 (Fontenot, J D et al. (1995) Proc. Natl. Acad. Sci. USA 92, 315-319) all show an increased β-turn propensity around GPGRAF [SEQ ID NO:5]. V3 peptides attached to filamentous bacteriophage fd viral coat protein pVIII (Jelinek et al., supra) adopted a double-turn structure similar to that observed in the crystal structure of the Fab 59.1-peptide complex (Ghiara et al., 1994, 1997, supra).

Crystal structures have been determined for complexes of V3_{MN} peptides with four different neutralizing murine mAbs—50.1 (Rini et al., supra) 59.1 (Ghiara et al., supra), 58.2 (Stanfield et al., supra) and 83.1—which were made by first immunizing mice with V3 peptides. The conformation of V3 peptides bound to mAb Fab fragments 50.1 (CKRIHIGPG [SEQ ID NO:6]), 59.1 (IHIGPGRAFYT [SEQ ID NO:7]), and 83.1 (KRIHIGPGRA [SEQ ID NO:8]) are all highly similar, with residues KRIHI [SEQ ID NO:9] forming an extended β-strand, immediately followed by a β-turn around GPGR [SEQ ID NO:3] (type II for 50.1 and 59.1, type I for 83.1). The peptide bound to Fab 59.1 continues with a type-I/I double bend consisting of a type I turn around GRAF [SEQ ID NO:11] and a type I turn around RAFY [SEQ ID NO:12]. The GPGR [SEQ ID NO:3] turn in the Fab 58.2 complex differs from that in the other V3 peptides, largely due to different torsion angles for the first Gly, which cause the peptide backbone to change direction with respect to the structurally-conserved KRIHI [SEQ ID NO:9] β-strand. Residues GPGR in this latter complex form a type I turn, and GRAF a type VIa turn.

The human mAb 447-52D (also abbreviated 447 or 447D herein) (IgG3, λ) was originally isolated from a heterohybridoma derived from peripheral blood mononucleocytes from a clade B HIV-1 infected individual (Gorny, M K et al. (1993) J. Immunol. 150, 635-643). 447-52D is one of the most broadly neutralizing and most potent anti-V3 antibodies that have been studied to date. It binds to intact virions from clades A, B, C, D, F, G and H (Nyambi, P N et al. (1998) J. Virol. 72, 9384-9391) and neutralizes primary isolates from several clade, including both X4 and R5 type viruses (Cecilia, D et al. (1998) J. Virol. 72:6988-6996; Conley, A J et al. (1994) J. Virol. 68:6994-7000; Fouts, T R et al. (1997) J. Virol. 71:2779-2785; Gorny, M K et al. (2002) J. Virol. 76:9035-9045; Hioe, C E et al. (1997) Int. Immunol. 9:1281-1290; Nyambi et al., supra; Verrier, F et al. (2001) J. Virol. 75:9177-9186). 447 recognizes the V3 loop; its core epitope has been mapped with overlapping peptides to the highly conserved V3 crown GPxR (residues 319-322) (Gorny M K et al. (1992) J. Virol. 66:7538-7542; Gorny et al., 1993, supra). Unlike most V3 antibodies, 447-52D can neutralize both X4 and R5 primary viral isolates correlating with its ability to bind V3 peptides with a wide range of sequence variability (Zolla-Pazner, S et al. (1999) J Virol 73:4042-4051.

447 binds to different V3 peptides with association constants ranging between 2×10⁵ and 10⁸ M⁻¹, the highest of which is only one order of magnitude lower than its affinity for the corresponding (intact) gp120 protein (VanCott, T C et al. (1994) J. Immunol. 153:449-459). Since 447-52D was elicited during the course of a natural HIV-1 infection and neutralizes a broad spectrum of HIV-1 isolates, it is believed to recognize a native V3 conformation. Consequently, the present inventors hypothesized that the structure of 447 complexed with V3 peptides serves as a reliable model for understanding the interactions between gp120 and V3-specific anti-HIV antibodies, and for identifying features of the surface of the V3 loop that interact with the chemokine receptors on target cells. An understanding of how 447-52D is able to effect such unusually broad neutralization of V3 could-facilitate design of a V3-related immunogen that may serve as either a protective or therapeutic vaccine for HIV-1 disease. The present invention is directed to a definition of the structure of a V3 epitope or epitopes formed when this peptide binds to the 447-52D antibody.

### SUMMARY OF THE INVENTION

It is now appreciated that, though the sequence in V3 is variable, the V3 loop is characterized by a constant size of 30-35 amino acids, a conserved type II β-turn at its tip, a disulfide bond at its base and a net positive charge (Kwong et al., 2000, supra). These structural constraints on the V3 loop appear to be imposed by the requirement for V3/chemokine receptor interaction (Hill, C M et al., 1997. J Virol 71:6296; Trkola et al., supra). This suggested to the present inventors that V3 must have conserved conformational aspects despite the sequence variation. This is borne out by reports that conserved elements in the V3 crown and stem are mandatory elements for coreceptor interaction (Wang, W K et al. (1999) Proc Natl Acad Sci USA 96:4558-62; Suphaphiphat, P et al., 2003, J Virol 77:3832; Cormier, E G et al., 2002, J Virol 76:8953). Cast in this new light, the present inventors predicted that antibodies to constrained V3 conformational epitopes would have potent and broad neutralizing activity.

The present inventors approach was to study the conformation of V3 peptides as they bind to broadly neutralizing human anti-V3 mAbs induced by natural infection. These studies, described here in part, and are the first to illuminate the structure of the V3 loop as it appears to the immune system in vivo. As discussed in detail below, the results of this analysis suggested that the V3 loop is a molecular mimic for the β-hairpin structures that appear in the physiologic ligands of the R5 and X4 receptors; these results suggest that the critical function of the V3 loop in binding to chemokine receptors dictates that it possess a limited number of conserved conformations.

The V3 mimetic immunogens may be used in a prime/boost immunization schedule of a mammal preferably a human or for further analytical purposes for rabbits to focus the antibody response on this neutralizing epitope and induce antibodies that will inhibit V3/coreceptor binding. This approach will optimally induce high levels of these antibodies. One way to accomplish this is to administer the constrained peptide composition of the present invention as boosters after binding them to an immunogenic carrier molecule and eliciting a secondary antibody response to the V3 loop in subjects which had been primed with, for example, a gp120 DNA vaccine.

One goal of the present invention was to provide a method to identify, screen for, and/or design novel compounds that would serve as immunogens for stimulating the production of potent, broadly neutralizing antibodies against HIV-1 such as 447-52D. An important binding target for such antibodies is the V3 loop of the HIV-1 gp120 envelope glycoprotein.

Based on the crystal structure available at the time of this invention, one would expect difficulties in engineering a modified version of gp120 with a correctly folded V3 loop, while removing other epitopes. For example, at least 2 major β-hairpins would have to be deleted. Therefore, it is very likely that V3 would not remain correctly folded, since neighboring regions, or even more distant regions due to folding, may be necessary to preserve its structural integrity. This prompted the present inventors to employ a different approach, that of understanding the three-dimensional (3D) structure of V3 when it was bound to, and constrained by, a potent, broadly neutralizing human mAb.

The present inventors and their colleagues discovered the 3D structure of a V3_{MN} peptide (³⁰⁸YNKRKRIHI--GPGRAFYTTKNIIG³³² [SEQ ID NO:13] as it is recognized and bound by the HIV-1 neutralizing human mAb 447-52D, abbreviated herein as 447D or 447, or, more specifically bound to its Fv fragment (“447Fv”). (The “--” in the sequence above represents the position of a two residue insertion in the V3 loop of HIV-1 strain IIIB or V3_{IIIB}). Subsequently, they solved the 3D solution structure of V3_{IIIB} peptide (³¹⁰⁻³²⁸gp120_{IIIB}) bound to the 447-52D Fv. That peptide molecule had the sequence TRKSIRIQRGPGRAFVTIGK [SEQ ID NO:37]

The backbone of the V3_{MN} 447-bound peptide forms a β-hairpin with two anti-parallel β-strands linked by an inverse γ-turn. The N-terminal β-strand and four residues of the C-terminal β-strand contribute almost all the interactions between the V3_{MN} loop and the 447Fv, indicating that these residues are exposed, and able to participate in chemokine-receptor binding. The backbone of the bound V3_{IIIB} peptide also forms a β-hairpin with two anti-parallel β-strands each comprising 4 residues linked by a 7-residue loop. The N-terminal residues KSIRI [SEQ ID NO:15] of V3_{IIIB} and KRIHI [SEQ ID NO:9] of V3_{MN} adopt similar conformations. In both complexes the Lys and two Ile residues show extensive-interactions with the antibody and exhibit the same side-chain orientation. In contrast, the sequence of the V3_{IIIB} C-terminal β-strand FVTIG [SEQ ID NO:16] differs from the corresponding region of V3_{MN} in side chain orientation and in the residues that are involved in hydrogen bonding. However, despite these differences, both the V3_{MN} and the V3_{IIIB} β-hairpins are similar in conformation to a β-hairpin region of (a) CD8 and (b) the R5 chemokines MIP-1αMIP-1β and RANTES.

Notably, the β-hairpin conformation of a V3_{IIIB} peptide bound to a different mAb Fv fragment, 0.5β-Fv, solved by one of the present inventors and his coworkers (Tugarinov et al., supra) is, according to the present invention, different, resembling a 1-hairpin in the chemokine Stromal Cell-Derived Factor-1 (SDF-1)) which is a X4 ligand (Bleul, C C et al., Nature (1996) 382:829-833; Oberlin E, Nature (1996) 382:833-835). These results suggested that these two distinct β-hairpin conformations of the V3 loop are responsible for co-receptor selectivity.

The present invention is therefore directed to compositions that comprise a peptide or peptidomimetic compound that is constrained to mimic the 3D conformation of the V3 peptide as it is bound to a neutralizing antibody binding site, preferably that of 447-52D but also of others such as the murine mAb 0.5β mAb.

One preferred embodiment is a peptide and/or mimic of the conformation of V3_{MN} bound to 447 Fv but with sequence characteristic of R5 viruses. One such peptide is termed “R5A” because it represents one type of constrained structure that binds to the R5 co-receptor. The other embodiment is a peptide and/or mimics of the V3_{IIIB} peptide conformation when bound to 447 Fv but with sequence characteristic of R5 viruses. Such a peptide is termed “R5B” because it represents a second type of constrained structure that binds to the R5 co-receptor. The R5A and R5B peptides differ in their C-terminal conformation and in the hydrogen bond network formed as a result of the constraints.

The present inventors also analyzed the structure of two self-constrained synthetic cyclic peptides which were designed to mimic antibody-constrained V3_{MN} (the R5A form that mimics the conformation of V3_{MN} bound to 447 Fv) and antibody-constrained V3_{IIIB} (the R5B form that mimics the conformation of V3_{IIIB} bound to 447 Fv). They were based on the V3 loop consensus sequence of R5 viruses, as represented by the JRFL strain which has the sequence at residues 308-329: ³⁰⁸NNTRKSIHI--GPGRAFYTTGE³²⁹ [SEQ ID NO:59]. For information on JRFL, see Myers et al., supra).

The first of these novel self-constrained cyclic peptides (see Example X) termed R5A-M1 (mimic #1 of one of two types of R5-binding peptides, R5A) includes two specified disulfide bridges formed by four Cys residues substituted into the sequence of V3_{JRFL}. The sequence and of R5A-M1 is as shown below (aligned with the V3_{JRFL} sequence). Unlike HIV_{MN}, HIV_{JRFL} is a R5 virus. R5A-M1 peptide is a first generation constrained peptide consisting entirely of natural L-amino acids made according to this invention and has the following sequence with disulfide bridges indicated:

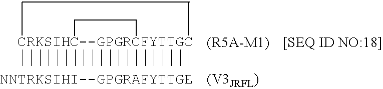

A distinct structure for an R5 ligand is termed R5B. Two constrained peptides having the R5B conformation, R5B-M1 and R5B-M2, are described in Example X. Although NMR analysis of these molecules has not yet been completed, these peptide are believed to be mimics of peptides/proteins with the R5B conformation.

The second of these novel self-constrained cyclic peptides (see Example X) is designated X4-M1. This name reflects the fact that this peptide, albeit based on the sequence of V3_{JRFL} loop of an R5 virus, mimics an X4-type conformation, that of V3_{IIIB} as bound to and constrained by mAb 0.5β. Like R5A-M1 above, X4-M1 includes two specified disulfide bridges formed by four Cys residues substituted into the sequence of V3_{JRFL}. The sequence of X4-M1 with disulfide bridges indicated is shown below (aligned with the V3_{JRFL} sequence).

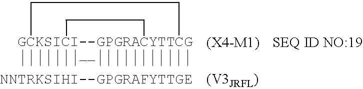

These self-constrained cyclic peptides and other peptide mimetics designed according to the present invention are used as models for further steps in refinement and modification of the design of additional mimetic molecules with improved properties (such as higher binding affinity to broadly reactive neutralizing anti-HIV antibodies, to HIV-1 co-receptors, etc.).

The present inventors have thus provided several new peptide conformations, and two novel constrained peptides each comprising two internal disulfide bonds, that are useful for the design of novel anti-HIV agents, or, in the case of these new peptides can themselves be implemented in several distinct ways in the prevention or treatment of HIV disease.

First, such constrained peptides or peptidomimetics having the same or very similar conformations are used as immunogens to induce broadly neutralizing antibodies with properties like the human mAb 447 that are active against the broadest possible range of HIV-1 isolates or clades.

Also provided are immunogenic or vaccine compositions comprising such peptides preferably conjugated or fused to immunogenic proteinaceous carriers. Immunogenic compositions preferably comprise adjuvants as nonspecific stimulators of immune reactivity in an immunized subject. Such antibodies can either protect a subject from an initial HIV infection, or, if induced in an infected subject, inhibit viral spread within the patient and between individuals. In another embodiment a high titered purified antibody can be used to transfer passive immunity to an infected or high risk subject.

In another embodiment, the constrained peptides can be used as antagonists that inhibit interactions between HIV virions and co-receptors on target lymphocytes (generally R5 receptors) or target cells of the monocyte/macrophage or other myeloid lineage (generally X4 receptors). Such inhibition can suppress viral infectivity and intercellular viral spread by reducing the ability of virions to bind productively to target cells.

The present invention also includes pharmaceutical and/or immunological compositions of the above compounds and methods for using the compositions in inducing anti-HIV-1 immunity and/or in treating or preventing HIV-1 infections by inhibiting viral spread. A preferred use of such an antagonist would be to treat a subject very soon after potential exposure to HIV-1 (such as (i) a health care worker accidentally exposed to the virus, or (ii) after unprotected sex with an infected individual).

The present compositions may be converted into reagents that are useful in isolating molecules or cells which bind to the constrained peptides or mimics, i.e., antibodies, B lymphocytes with surface immunoglobulins of the appropriate specificity, chemokine receptor molecules and cells bearing the chemokine receptors.

Definition of the peptide conformations that are “adapted” to fit the antigen binding pocket of broadly reactive neutralizing antibody are based on NMR structures for each peptide, which appear as X, Y, and Z orthogonal coordinates in Tables 3-6. The NMR constraints and structural statistics for the refined peptide structures are shown in Tables 1 and 2. Similar information derived from X-ray crystallographic studies are also presented briefly in Example VII. The X ray diffraction results confirm the structural parameters first obtained by NMR analysis. The inventors' NMR analysis has identified two subtypes of V3 β hairpin structures (termed R5A and R5B) that differ in the C-terminus of the P strand (residue positions approximately 324-327 of the gp120 sequence). X-ray analysis has the added advantage of providing information that better defines the fine structure of the antibody cleft and the residues therein that contact the amino acids of the peptide/mimetic.

Other embodiments of the invention are directed to compositions that include chimeric or fusion proteins in which a constrained V3 peptide structure is achieved by substituting a V3 sequence into a region of a protein that has a β-hairpin structure that closely resembles that of V3 bound to an antibody such as 447, so that the protein can accommodate the V3 peptide with minimal clashes. Protein database searches by the present inventors and colleagues have uncovered several such candidate proteins that are characterized by a relatively small root mean square deviation (rmsd) from the parameters of the 447-constrained structure of the V3_{MN} peptide. Similar searches are carried out using the coordinates of the 447-constrained V3_{IIIB} peptide, or the free R5A-M1, R5B-M1/M2 and X4-M1 peptides in solution. In polypeptides having these requisite characteristics, the structure surrounding the β-hairpin is expected to accept and accommodate the V3/mimic sequence, and to provide some of the necessary bond forces to constrain the grafted residues in the proper energy-minimized form. Parameters of such structures include torsion angles that do not exceed a certain limit, e.g., 5°, and preferably, no NOE violations, and a rmsd value of the backbone structure that does not exceed 2 Å, preferably not exceeding 1.8 Å, more preferably not exceeding 1.5 Å.

More specifically, the present invention is directed to a composition comprising an isolated peptide molecule or an isostere or non-peptidic molecular mimetic thereof, which peptide, isostere, or mimetic mimics the 3D atomic structural conformation, preferably NMR structure, of a V3 loop peptide of HIV-1 envelope glycoprotein gp120 that is bound to, and constrained by a broadly neutralizing anti-V3 mAb, preferably human mAb 447-52D and murine mAb 0.5β, or an antigen binding fragment of the in mAb, wherein the constrained V3 loop peptide differs in conformation from the same V3 loop peptide when it is in free form.

Preferably the conformation is defined by a set of NMR structure coordinates having a rmsd of not more than about 2 Å, preferably about 1.8 Å, more preferably about 1.5 Å, n the backbone atoms from the sets of structure coordinates in Table 3 or Table 4.

Preferably the V3 loop peptide has the sequence of a segment within the V3 loop of the gp120 protein of HIV-1_{MN} or HIV-1_{IIIB}.

In one embodiment of the composition, the isolated peptide has an amino acid sequence that is
(a) a substitution variant of no more than 6 substituted amino acid residues of KSIHIGPGRAFYTT [SEQ ID NO:17], KRIHIGPGRAFYTT [SEQ ID NO:20] or KSIRIQRGPGRAFVTIG [SEQ ID NO:28], wherein preferably the substitutions are not of amino acids that reflect the genetic variability of the V3 regions among viral strains, but rather are substitutions for purposes of engineering the constrained peptide;
(b) a terminal or internal addition variant of no more than about 20 added amino acid residues of KSIHIGPGRAFYTT [SEQ ID NO:17], KRIHIGPGRAFYTT [SEQ ID NO:20] or KSIRIQRGPGRAFVTIG [SEQ ID NO:28]; or of the substitution variant of (i); or
(c) a deletion variant of no more than 3 deleted residues of KSIHIGPGRAFYTT [SEQ ID NO:17]. KRIHIGPGRAFYTT [SEQ ID NO:20] or KSIRIQRGPGRAFVTIG [SEQ ID NO:28].

The isolated peptide above is preferably a cyclic peptide, preferably constrained by one or two internal disulfide bridges. Preferred disulfide constrained peptides are

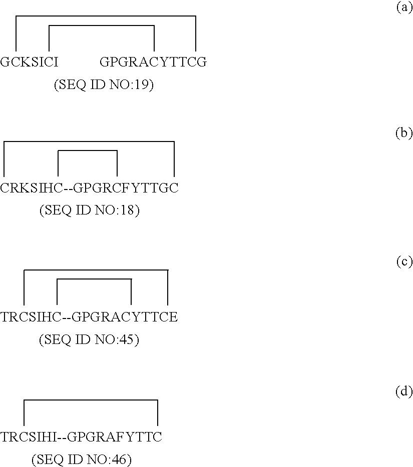

Preferably, in the above composition, the isolated peptide binds selectively to R5 or X4 chemokine receptors.

The isolated peptide preferably binds to mAb 447-52D or an antigen binding fragment thereof with an affinity characterized by a K_{d} of at least about 100 nM, preferably at least about 10 nM, more preferably at least about 1 nM.

Also provided is a composition comprising a complex of human mAb 447-52D or an antigen binding fragment thereof and a peptide of the V3 loop region of HIV-1 envelope glycoprotein gp120, or an isostere or mimic thereof, wherein the 3D conformation of the antibody-complexed peptide is conformationally constrained and altered by the antibody so that it differs from the 3D atomic structure of the same V3 loop peptide when it is in free form.

The complex may be one in which the peptide has the properties recited above that characterize the isolated peptide.

The invention is also directed to method of identifying from among a plurality of existing compounds a molecule that is useful as an HIV-1 V3 loop immunogen or as an inhibitor of binding of HIV-1 to a chemokine receptor/HIV-1 co-receptor on the surface of a receptor-bearing target cell, which method comprises:
(a) either or both of:
   (i) subjecting to NMR analysis and solving the structure of a peptide that is specifically bound to mAb 447-52D and/or mAb 0.5β, or to an antigen binding fragment of the mAb with an affinity of at least 1 μM; and
   (ii) employing the NMR coordinates listed in any of Tables 3-6; and
(b) screening the plurality of compounds for a compound that has the following characteristics:
   (i) a conformation defined by NMR structure coordinates having an rmsd of not more than about 2 Å, preferably f not more than about 1.8 Å, more preferably not more than about 1.5 Å, in the backbone atoms from NMR coordinates determined in step (a)(i) or the NMR coordinates listed in any of Tables 3-6;
   (ii) specifically binds to mAb 447-52D or 0.5β with an affinity of at least about 100 mM
   (iii) selectively binds to either or both of the chemokine receptors R5 and X4 with an affinity of at least 1 μM as measured in a receptor binding assay;

which screening steps and characteristic determination is performed by computational means, by experimental means, or by both, and

which molecule is identified to be useful as an immunogen or inhibitor if it has the characteristics of (b)(i), (ii) and (iii), and
(1) if it is a natural peptide comprising L-amino acids, the molecule does not have a sequence that is a native, uninterrupted sequence of
   (A) an HIV V3 loop peptide epitope, or
   (B) MIP-1α, MIP-1β, RANTES, SDF-1 or a fragment thereof that binds to the receptor,
   or
(2) is a partial peptide or non-peptidic peptidomimetic compound.

Preferably, the screening in (b) above is for selective binding to R5 chemokine receptors or to X4 receptors and specific binding to the mAbs is with an affinity of at least about 10 nM.

Another embodiment provides a method of designing a molecule that is useful as an HIV-1 V3 loop immunogen or as an inhibitor of binding of HIV-1 to a chemokine receptor/HIV-1 co-receptor on the surface of a receptor-bearing target cell, which method comprises:
(a) either or both of:
   (i) subjecting to NMR analysis and solving the structure of a peptide that is specifically bound by mAb 447-52D and/or mAb 0.5β or an antigen binding fragment of the mAb with an affinity of at least 1 μM; and
   (ii) employing the NMR coordinates listed in any of Tables 3-6; and
(b) determining conformational parameters of the molecule being designed such that the molecule has a β-hairpin structure and a hydrogen bonding network that result in a conformation defined by NMR structure coordinates having an rmsd of not more than about 2 Å in the backbone atoms from NMR coordinates determined in step (a)(i) or listed in any one of Tables 3-6; and
(c) if the molecule is a peptide or partial peptide, selecting amino acids and a sequence that will result in a molecule with the conformational parameters of (b),
   thereby designing the molecule.

Also provided is a method for making a molecule that is useful as an HIV-1 V3 loop immunogen or as an inhibitor of binding of HIV-1 to a chemokine receptor/HIV-1 co-receptor on the surface of a receptor-bearing target cell, which method comprises:
(a) designing the molecule as above;
(b) selecting a synthetic process that will stabilize the β hairpin structure and hydrogen bonding network, and
(c) employing the synthetic process of (b) to synthesize the molecule.

The method may further comprise:
(d) testing the molecule produced in step (c) for one or both of (i) specific binding to an anti-HIV-1 V3-specific neutralizing mAb or antigen binding fragment thereof, preferably 447-52D or 0.5β mAb; and (ii) selective binding to either or both CCR5 and CXCR4 chemokine receptors.

The method for making the molecule preferably further comprises selecting, as useful, a molecule having the following characteristics:
(i) specifically binds binding to the mAb or fragment with an affinity of at least about 100 nM; and
(ii) selectively binding to the receptor with an affinity of at least 1 μM as measure in a receptor binding assay.

The β-hairpin structure is preferably stabilized by internal disulfide linkages between Cys residues, internal hydrazone linkages or backbone cyclization using disubstituted amino acids.

The above method may further comprise the step of testing the molecule for one or more of the following activities:
(a) inhibition of binding of gp120 or the V3 loop peptide to the mAb or fragment;
(b) inhibition of binding of MIP-1α, MIP-1β, RANTES, SDF-1 or a fragment thereof to the receptor
(c) inhibition of gp120 or HIV-1 binding or attachment to the chemokine receptor/co-receptors or to target cells expressing the receptors; and
(d) inhibition of HIV-1 infectivity of cells in culture;

The method may further comprise selecting, as useful, a molecule that scores positive for one of more of the inhibitory activities.

The invention is also directed to a composition that is useful as an HIV-1 V3 loop immunogen or as an inhibitor of binding of HIV-1 to a chemokine receptor/HIV-1 co-receptor on the surface of a receptor-bearing target cell, comprising a molecule designed in accordance with any of the above methods.

Also provided is an immunogenic composition for induction of an anti-HIV-1 antibody response specific for a V3 loop epitope, comprising (a) any of the above compositions wherein isolated peptide molecule, isostere or non-peptidic molecular mimetic is preferably fused or conjugated to an immunogenic carrier such as tetanus toxoid; and (b) an immunologically acceptable excipient.

A pharmaceutical composition useful for blocking the interaction of HIV-1 with an R5 or X4 co-receptor and thereby inhibiting HIV-1 infectivity, comprises
(a) a composition as above; and
(b) a pharmaceutically acceptable carrier or excipient.

The pharmaceutical composition may further comprising one or more agent effective against HIV-1 infection or which treats symptoms associated with HIV-1 disease.

A method for inducing in a subject an anti-HIV-1 neutralizing antibody response specific for a V3 loop epitope, comprising administering to the subject the above immunogenic composition. The subject is one who is infected with, or at risk of infection with, HIV-1.

A method of inhibiting infection by HIV-1, comprising providing to cells at risk for the infection and infection-inhibiting effective amount of the above composition.

Also provided is a method of preventing an HIV-1 infection in an uninfected subject at risk for such infection or for inhibiting viral spread and disease progression in an infected subject, comprising administering to a subject in need of the prevention or inhibition an effective amount of the above pharmaceutical composition.

The invention is directed to a the use of a composition as above in the manufacture of a medicament for use in treating or preventing HIV-1 infection.

One embodiment is a computing platform for generating a 3D model of a constrained HIV V3 loop peptide when it is bound to 447-52D or 0.5β mAb or to an antigen binding fragment thereof, which computing platform comprises:
(a) a data-storage device storing data comprising a set of structural coordinates defining the structure of the constrained V3, and
(b) a data processing unit for generating the 3D model from the data stored in the data-storage device.

Also included is a computer generated model representing the conformationally constrained structure of a V3 loop peptide that is bound to 447-52D or 0.5β mAb or to an antigen binding fragment thereof, the computer generated model having a 3D atomic structure defined by a set of NMR coordinates set out in any of Tables 3-6

The invention is also directed to a computer readable medium comprising, in a retrievable format, data that includes a set of structure coordinates defining a 3D structure of a V3 loop peptide that is conformationally constrained by being bound to 447-52D or 0.5β mAb or to an antigen binding fragment thereof.

In the computer readable medium, the structure coordinates defining a the 3D structure preferably correspond to a set of NMR coordinates which have an rmsd of not more than about 2 Å in the backbone atoms from the sets of structure coordinates in any of Tables 3-6.

### BRIEF DESCRIPTION OF THE DRAWINGS

Certain figure descriptions below refer to colors not shown in the present figures—but rather represented by black/white/gray. The identical color-containing figures are found in a related publication by the present inventors and colleagues, Sharon, M et al. (2003) “Alternative conformations of HIV-1 V3 loops mimic β hairpins in chemokines, suggesting a mechanism for coreceptor selectivity.” Structure 11:225-236, which is incorporated by reference in its entirety.

FIG. 1 shows NMR mapping of the 447-52D epitope. FIG. 1A is a diagram showing the variations in the ¹H^{N}/¹⁵N cross-peak intensities of a ¹⁵N TROSY-HSQC spectrum recorded using uniformly ¹⁵N-labeled V3_{MN} peptide bound to unlabeled 447Fv. FIG. 1B is a diagram showing the variations of the ¹⁵N(¹H) T₂ relaxation times of the bound V3_{MN} peptide along the peptide sequence. The asterisk denotes an overlap between residues K310 and K328.

FIGS. 2A-2C show the solution structure of the V3_{MN} epitope (³¹²⁻³²⁷gp120_{MN}) bound to the 447-52D Fv fragment. FIG. 2A shows backbone superposition of 29 lowest energy structures. FIG. 2B is a ribbon diagram of the energy-minimized average structure; the terminal residues of the β-sheets are numbered. FIG. 2C is a stereo representation of V3_{MN} bound to the 447Fv showing sidechain interactions and hydrogen-bonds within the peptide (in red in Sharon et al., supra). Side chains pointing out from the page are in yellow (in Sharon et al.) and sidechains pointing inward are in green (in Sharon et al.).

FIG. 3 shows intermolecular interactions of the V3_{MN} peptide with the 447Fv. The total number of intermolecular interactions observed in the NOESY spectra is shown for each residue of the epitope.

FIGS. 4A-4C show structural homology of the V3 β-hairpins with CD8, MIP-1α, MP-1β, RANTES and SDF-1. Backbone superposition of the 447Fv bound V3_{MN} peptide (blue in Sharon et al.) with CD8 (red in Sharon et al.) (FIG. 4A) and CCR5 natural ligands MIP-1α (green in Sharon et al.), MIP-1β (magenta in Sharon et al.) and RANTES (yellow in Sharon et al.) (FIG. 4B). The sidechains of I314, I316 and R322 in V3_{MN} and their equivalents in CD8 (L50, L52 and K56), MIP-1α (I40, L42, R45 or R47) and RANTES (V40, V42, K45 or R47) are shown in FIG. 4B. FIG. 4C is a backbone superposition of the 0.5β Fv bound V3_{IIIB} peptide (cyan) with SDF-1 (purple). Sidechains of I314 and I316 of V3_{IIIB} and A40 and L42 in SDF-1 are shown. The sequence alignment of the V3 peptides with the homologous β-hairpins in CD8, MIP-1α, RANTES and SDF-1 are shown below.

| | | SEQ |
|---|---|---|
| Protein/peptide | Sequence | ID NO: |
| V3_{MN} | -³¹²KRIHIGPGRAFYTT³²⁷ | 20 |
| CDS | -⁴⁸FLLYSQNKPKAAE⁶¹ | 21 |
| MIP-1α | -³⁸GVIFLTKRSRQVCA⁵¹ | 22 |
| MIP-1β | -³⁹AVVFQTKRSKQVCA⁵² | 10 |
| RANTES | -³⁸AVVFVTRKNRQVCA⁵¹ | 23 |
| V3_{IIIB} | -³¹²KSIRIQRGPGRAFVTI³²⁷ | 24 |
| SDF-1 | -³⁸IVARLKN--NNRQVCI⁵¹ | 25 |

FIG. 5 shows the structure of the V3_{MN} peptide bound to the 447Fv compared to the structures of V3 peptides bound to antibodies raised originally against a 40-residue cyclic peptide. FIG. 5 is backbone superposition of the bound V3_{MN} peptides in complex with the 447Fv (blue in Sharon et al.) or with three other mAbs—50.1 (yellow in Sharon et al.)), 59.1 (green in Sharon et al.)) and 58.2 (red in Sharon et al.)). The list below shows the epitopes recognized by each antibody and the type of turn the bound peptide forms. Residues in β-strands are underlined. The residues shown in bold form the turns. Also included in the table is the sequence of the V3 peptide from HIV_{IIIB} which was studied when bound to the 0.5β mAb and when bound to the 447Fv.

| | | | | |
|---|---|---|---|---|
| | | Epitope & | SEQ ID | |
| Antibody | Peptide | Structure | NO: | Turn |

| | | | | |
|---|---|---|---|---|
| 447 | V3_{MN} | KRIHI--GPGRAFYTT | 20 | Inverse γ |
| 50.1 | V3_{MN} | KRIHI--GPG | 26 | Type II β |
| 59.1 | V3_{MN} | HI--GPGRAFYT | 1 | Type II- |
| | | | | Type Iβ |
| 58.2 | V3_{MN} | RIHI--GPGRAFY | 27 | Type I, |
| | | | | Type 1β |
| 0.5β | V3_{IIIB} | KSIRIQRGPGRAFVTI | 24 | Type VI β |
| 447 | V3_{IIIB} | KSIRIQRGPGRAFVTIG | 28 | Not |
| | | | | defined |

FIGS. 6A-6E show an analogy between the dual β-hairpin conformations formed by V3 loops and the conformations of the homologous β-hairpins in MIP-1α and SDF-1. FIG. 6A is a backbone superposition of the homologous β-hairpins in SDF-1 (yellow in Sharon et al.)), V3_{IIIB} (green in Sharon et al.)), MIP-1α (red in Sharon et al.)) and 447Fv-bound V3_{MN} (blue in Sharon et al.) obtained by best superposition of triads I40-R41-L42 (MIP-1α), A40-R41-L42 (SDF-1), I314-H315-I316 (V3_{MN}) and I314-R315-I316 (V3_{IIIB}). FIGS. 6B and 6C are ribbon diagrams of MIP-1α and SDF-1, respectively. The β-hairpins homologous to V3 are shown in a space-fill view, while nearby residues are shown in sticks. FIGS. 6D and 6E show space fill representations of V3_{MN} and V3_{IIIB}, respectively. The residues of the above triads are highlighted, residues I40 and L42 in MIP-1α, A40 and L42 in SDF-1, I314 and I316 in both V3_{MN} and V3_{IIIB} are colored light green in Sharon et al. The aromatic residues F41 in MIP-1α and H315 in V3_{MN} are dark green in Sharon et al. and R41 in SDF-1 and R45 in MIP-1α are blue in Sharon et al.

FIGS. 7A and 7B show in ribbon or partial space filling format that the epitope portion of the V3_{MN} structure (as bound by 447) (green in Sharon et al.) disclosed herein superimposes well to the backbone of a β-hairpin of RANTES (purple in Sharon et al.). However, about 6000 other experimental β hairpin structures superimpose even better. Adding the sequence filter “IxxGPGxxxYxT” [SEQ ID NO:29] brings RANTES from rank 6026 to 17.

FIGS. 8A-8C show the hairpin of defensin-o (pdb code 1dfn) superimposed with V3_{MN} and V3_{IIIB} structures based on NMR analysis of these HIV-1 peptides bound to mAbs 447 and 0.5β, respectively.

FIGS. 9A-9B depict the solution structure of the V3_{IIIB} epitope (³¹²⁻³²⁸gp120B) bound to the 447-52D Fv. FIG. 9A shows the backbone superposition of 29 lowest-energy structures. FIG. 9B is a ribbon diagram of the energy-minimized average structure (see Tables 2 and 4). The terminal residues of the β strands are numbered.

FIG. 10 shows the hydrogen bond network within the V3_{IIIB} peptide. The residues forming the two β strands are shown.

FIG. 11 compares the structures of the V3_{IIIB} and V3_{MN} peptides when bound to 447 Fv. The diagram shows the backbone superposition of the N-terminal residues (³¹²⁻³¹⁶gp120) and the side chains of K312, I314, and I316.

FIG. 12 shows the structure of the V3_{IIIB} peptide when bound to the 447 Fv compared with the structure of the V3_{MN} peptide when bound to 447 Fv and V3_{IIIB} bound to 0.5β mAb. Backbone superposition of the N-terminal segment (³¹²⁻³¹⁶gp120) of 447-V3_{IIIB} (black), 447-V3_{MN} (white), and 0.5β-V3_{IIIB} (gray).

FIGS. 13A/1-13A/3) and 13B/1-13B/3 show a space-filling representation of the complexes V3_{IIIB}-447, V3_{MN}-447 and V3_{IIIB}-0.5β. In FIG. 13A, all peptide were aligned with their N-terminal half toward the front of the sheet. Residues ³¹²K-I-I³¹⁶ of the N-terminal region are shown in white. (/1) V3_{MN}-447, (/2) V3_{IIIB}-447, and (/3) V3_{IIIB}-0.5β. In FIG. 13B, all peptides were aligned with their C-terminal half toward the front of the page. Residues ³²⁴F-T-G³²⁸ of the C-terminal region are shown in white. (/1) V3_{MN}-447, (/2) V3_{IIIB}-447, (/3) V3_{IIIB}-0.5β.

FIGS. 14A-14C show 2D diagrams of the secondary structure and hydrogen bonding network (NMR analysis) of V3_{IIIB} peptide when bound to 0.5β Fv (FIG. 14A), V3_{IIIB} peptide when bound to 447 Fv (FIG. 14B) and V3_{MN} peptide when bound to 447 Fv (FIG. 14C)

FIGS. 15A-15D shows models of the structures of a chimeric protein based on the Bowman-Birk trypsin inhibitor (BBI) grafted with V3_{MN}. BBI preserves critical interactions with mAb 447 without introducing steric clash with the antibody.

### DESCRIPTION OF THE PREFERRED EMBODIMENTS

It is now appreciated that, though the sequence of the V3 region of HIV-1 gp120 is variable, the V3 loop is characterized by a constant length of 30-35 amino acids, a conserved β-turn at its tip, a disulfide bond at its base and a net positive charge (Kwong et al., 2000, supra). These structural constraints on the V3 loop in the protein appear to be imposed by the requirement for V3/chemokine receptor interaction (Hill, C M et al., 1997. J Virol 71:6296; Trkola et al., supra). Based on this, the present inventors conceived that the conformation of V3 must be relatively conserved despite the variation in its amino acid sequence. This conception is supported by reports that conserved elements in the V3 crown and stem are mandatory for interaction with the co-receptor (Wang et al., supra; Suphaphiphat, P et al., 2003, J Virol 77:3832; Cormier, E G et al., 2002, J Virol 76:8953). Cast in this new light, the present inventors conceived that antibodies to constrained V3 conformational epitopes would have potent and broad neutralizing activity.

The present inventors approach was to study the conformation of V3 peptides as they bind to broadly neutralizing human anti-V3 mAbs induced by natural infection. These studies, some of which are described herein, are the first to illuminate the structure of the V3 loop as it “appears to the immune system” in vivo. The results of this analysis indicated that the V3 loop is a molecular mimic for the β-hairpin structures that appear in the “physiologic” ligands of the R5 and X4 co-receptors/chemokine receptors. According to this invention, the critical function of the V3 loop in binding to chemokine receptors dictates that it possess a limited number of conserved conformations.

The present inventors have used NMR analysis to define the solution structure of the HIV-1 V3_{MN} and V3_{IIIB} peptides when they are bound to a potent neutralizing human mAb, 447. The uniqueness of this mAb is that it is derived from an antibody produced in an infected human responding to HIV-1 virions, rather than being induced artificially by isolated gp120 protein or by relatively short synthetic V3 peptides. Moreover, the antibody specificity appears to be directed to a conformational, rather than a linear, epitope. The inventors conceived that by understanding the structure of these peptides induced by binding to 447, it would be possible to design improved immunogens that, when administered to a subject, are far more likely to induce neutralizing mAbs like 447 characterized by both high potency and broad reactivity.

Further, the present inventors discovered that the structure of such bound V3 peptides resembles the “analogous” structures of chemokines. Because the cellular receptors for chemokines are HIV-1 co-receptors, artificially constrained peptides and other molecules that are partially peptidic or non-peptidic in nature can act as mimics of 447 mAb-constrained V3_{MN} and V3_{IIIB} conformations, and are therefore useful as antagonists for the chemokine receptors R5 and X4 that could inhibit virus binding by competitive binding and/or by inducing receptor internalization and loss. Thus, according to this invention, administration of such constrained peptides and isosteres or mimics thereof to a subject interferes with the infection and with spread of the virus from cell to cell.

It has generally been accepted in the art that the tip of the V3 loop is made up of 4 residues (GPGR) so that design of mimics would be designed around that feature. However an important discovery by the present inventors was the existence of (at least) two different structures of the antibody-constrained V3 loop that are related to the structure of R5 chemokines. The first of these, termed R5A is indeed based on the GPGR turn (as exemplified by the conformation adopted by V3_{MN} when bound to 447 Fv. The second structure, designated R5B, and exemplified by the conformation of V3_{IIIB} bound to 447 Fv has a conformation with a five residue tip, made up of GPGRA [SEQ ID NO:58]. In this conformation, the network of hydrogen bonding that stabilizing the β-hairpin has been shifted one residue or register “to the right” (towards the C-terminus), as described in more detail in the Examples and Table 4). This discovery is important because the homolgous loop in R5 chemokines also has 5 residues. In the absence of knowledge of the existence of this 5 residue V3 loop tip, such a homology could not have been appreciated. This relationship was not known and the significance heretofore had not been appreciated. Thus, structures designed to resemble the conformation defined by the 447-bound V3_{IIIB} peptide are expected to be closer in conformation to the R5 cytokines and may therefore be better inhibitors at the R5 receptor and improved agents to prevent infection or retard disease progression of R5-tropic HIV-1 strains.

Similarly, the discovery herein of the homology between the conformation of V3_{IIIB} peptide bound to 0.5β Fv (known before) and that of the X4 chemokine SDF-1, permits design of better inhibitors at the X4 receptor and improved agents to prevent infection and/or retard disease progression of X45-tropic HIV-1 strains. See Example X and Table 5 for description of this conformation.

Additionally, the present peptides/mimics can be used as reagents or tools to isolate and characterize the binding sites of neutralizing antibodies, cell surface receptors including the R5 receptor or B cell surface immunoglobulin receptors, or to selectively enrich or deplete cells bearing such receptors.

Further, the V3 mimetic peptides and other mimics are employed as immunogens to induce broadly neutralizing anti-V3 antibodies in human or other animal. Thus, such molecules can induce a highly protective and/or therapeutic state of immunity mediated by neutralizing antibodies. Additionally, antibodies induced by such immunogens are useful for inducing a state of passive immunity against HIV-1. The immunogens may be used along with other (including less potent) HIV-1 vaccine compositions in a prime/boost immunization scheme in mammals, preferably humans. The immunogens may also be used for further analytical purposes in animals such as rabbits to focus the antibody response on this neutralizing epitope defined by the constrained V3 structure and induce antibodies that will neutralize virus. These approaches will optimally induce high levels of these antibodies. In one embodiment, the constrained peptide composition of the present invention is administered as a booster, preferably bound to an immunogenic carrier molecule such as tetanus toxoid, to eliciting a secondary (or higher) antibody response against the V3 loop in subjects which had been primed with, for example, a gp120 DNA vaccine.

### Chemical Stabilization of Peptides

The compositions of the present invention may be synthesized using ordinary skill in the art of organic synthesis and peptide synthesis. New methods for restricting the secondary structure of peptides and proteins are highly desirable for the rational design of therapeutically useful conformationally-restricted (or “locked”) pharmacophores. These applications are exemplified by an analogue of eel calcitonin, [Asu^{1,7}]-eel calcitonin, in which α-aminosuberic acid (Asu) replaces the cysteine residues at positions 1 and 7 (Morikawa, T. et al., Experientia 32:1104-1106 (1976)). This analogue had significant biological activity, leading the authors to conclude that the disulfide bond in calcitonin is not essential for biological activity as long as the specific conformation of the peptide is maintained by an intramolecular bridge.

The purely chemical approaches for restricting secondary structure often requires extensive multistep synthetic work (Olson, G. L., J. Am. Chem. Soc. 112:323 (1990)). An alternative approach involves installing covalent bridges in peptides. However, due to the sensitivity of the peptide backbone and side chains, this method necessitates careful protection/deprotection strategies. For example, this problem occurs in the preparation of polymethylene analogues of [Arg⁸]vasopressin in which x-aminosuberic acid (Asu) replaces the cysteine residues at positions 1 and 7 and in which the N-terminal amino group is removed (S. Hase et al., Experientia 25:1239-1240 (1969); S. Hase et al., J. Amer. Chem. Soc. 94:3590 (1972)), yielding deamino-dicarba-Arg⁸-vasopressin.

Covalent linkages can, in selected instances, be established using other chemical methods, for example, by lactam formation between carboxylic acid and amine side chains

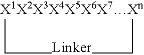

wherein n is preferably between 10 and 23 (i.e., a 10-mer to a 23-mer peptide) and the linker is optional, particularly if X¹ and Xⁿ are each Cys that naturally forms a disulfide linkage to secure the cyclic peptide.

In one embodiment, all of X¹ through Xⁿ represent L- or D-series amino acids corresponding to all or part of the V3 loop of the gp120 glycoprotein of an HIV-1 virus of the desired strain, tropism or co-receptor specificity. The present inventors prepared and analyzed a cyclic peptide from HIV-1_{JRFL} which is an R virus (V3_{JRFL}). Amino acid residues at the particular positions and the linker are selected according to criteria that constrain the peptide into a 3D conformation that mimics the conformation of V3_{MN} and/or V3_{IIIB} peptide when it is bound to the 447-52D human mAb, determined by NMR analysis as described and exemplified herein.

Nonlimiting examples of cyclic peptides using the sequence of V3_{MN} include:

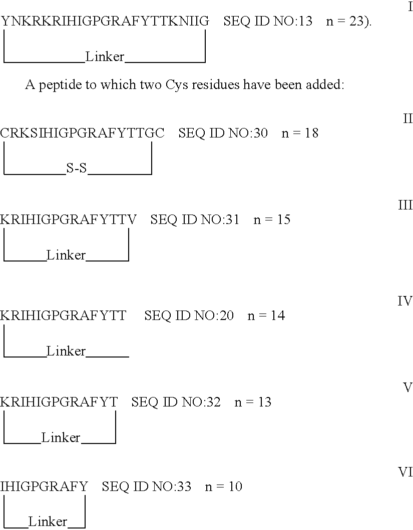

The cyclic peptide of formula II binds to 447 with 3-fold higher affinity than does the native V3_{MN} linear peptide.

Substitutions of both terminal residues with Cys, or additions of terminal Cys residues to a sequence, are one approach to achieve cyclization and contribute to the constraint of the peptide to the desired 3D parameters described herein. Coupled with additional substitutions or modification of sidechains or introduction other organic groups, a better fit can be achieved.

Examples of substitutions in a cyclic peptide of the formula III may be as follows:

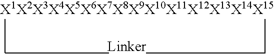

X¹ is K or R, X² is R or K, X³ is I, L or V, X⁴ is H, F or Y, X⁵ is I, L or V, X⁶ is G, X⁷ is P, X⁸ is G, X⁹ is R or K, X¹⁰ is A, X¹¹ is F, X¹² is Y, X¹³ is T, X¹⁴ is T, X¹⁵ is V [SEQ ID NO:34]. In another embodiment a Cys residue is added N-terminal to X¹ and C-terminal to X¹⁵. In yet another embodiment, X¹ and X¹⁵ are Cys.

Similar substitutions may be used in the shorter or longer V3_{MN} cyclic peptides/mimics. As discussed in the examples certain motifs are present in V3_{MN} and V3 sequences from other strains of HIV and from regions of chemokines that share structural similarity. Thus the I-x-I motif is present wherein the “x” residue was restricted to an aromatic residue, but not tryptophan. Ten β-hairpin structures were found to have the motif I-x-I with the following substitutions: (I/L/V)(H/F/Y)(I/L/V).

Moreover a basic residue is found separated from the (I/L/V)(H/F/Y)(I/L/V) motif by two or three residues, resulting in the following motifs:

| | |
|---|---|
| (I/L/V)-(H/F/Y)-(I/L/V)-x-x-x-(R/K) | [SEQ ID NO:35] |
| and | |
| (I/L/V)-(H/F/Y)-(I/L/V)-x-x-(R/K). | [SEQ ID NO:36] |

The sequence alignment of MIP-1α and RANTES chemokines with the V3_{MN} peptide is shown below as is the co-receptor specificity of the viral strain/receptor specificity of the chemokine:

| | | | |
|---|---|---|---|
| V3_{MN} | KRIHIGPGRAFYTTV | X4/R5* | SEQ ID NO:31 |
| CD8 | FLLYLSQNKPKAAE | | SEQ ID NO:21 |
| MIP1α | GVIFLTKRSRQVCA | R5 | SEQ ID NO:22 |
| RANTES | AVVFVTRKNRQVCA | R5 | SEQ ID NO:23 |

(* Although the sequence of the V3_{MN} is of an X4 virus, when bound to 47 Fv it adopts an R5 conformation)
In the I-x-I motif of the V3 peptide of IIIB, x is R (Arg) as shown below, and is aligned with the sequence of chemokine SDF-1

| | | | |
|---|---|---|---|
| V3_{IIIB} | KSIRIQRGPGRAFVTI | X4 | SEQ ID NO:24 |
| SDF-1 | IVARLKN--NNRQVCI | X4 | SEQ ID NO:25 |

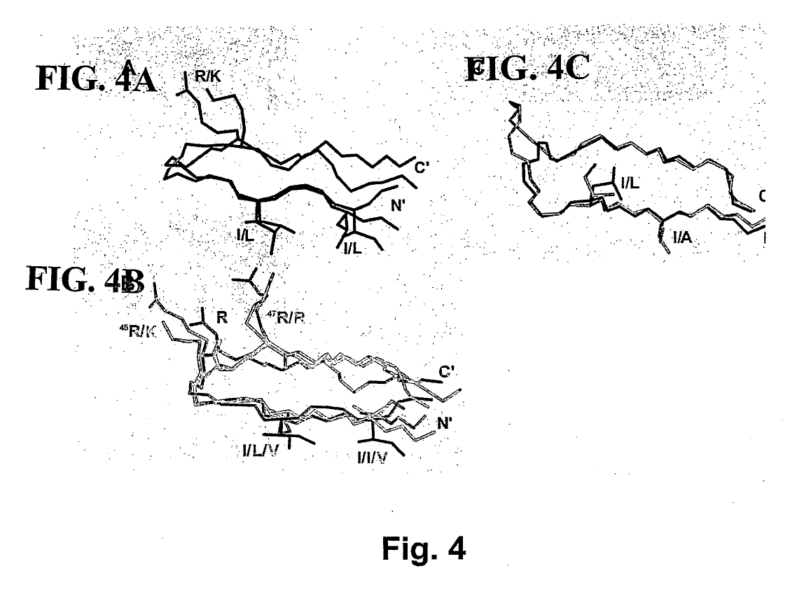

Five out of the above seven peptides bind to CCR5. Although the basic amino acid in RANTES and MIP-1α is separated by two residues from the (I/L/V)(H/F/Y)(I/L/V) motif (separated by three residues in V3_{MN}), the side chain of the basic residue points in the same direction in the three proteins. The overall topology of the (I/L/V)(H/F/Y)(I/L/V) tripeptide is similar, as shown in FIG. 4B.

Linker groups in the above cyclic peptide may include one or more amino acids or an aliphatic chain comprising carbon and hydrogen atoms, and may include carbonyl and amine groups as well. A linking unit or linker is one that creates a linear dimension between the C^{α} carbon of amino acid X¹ and the C^{α} carbon of the other “terminal” amino acid that permits the cyclic peptide to fit optimally to the NMR coordinates described herein of, for example, V3_{MN} or V3_{IIIB} bound to 447. Examples of linker groups designated L1 through L15 are:

| | |
|---|---|
| L1 | —CO—CH₂—NH—CO—CH₂—CH₂—CH(CO—NH—CH₂—CO—NH₂)—NH— |
| L2 | —CO—CH₂—NH—CO—CH₂—CH₂—CH(CO—NH—CH(CH₂SH)—CO—NH₂)—NH— |
| L3 | —CO—CH(CH₂SH)—NH—CO—CH₂—CH₂—CH(CO—NH—CH₂—CONH₂)—NH— |
| L4 | —CO—CH₂—NH—CO—CH₂—CH₂—CH(CO—NH—CH(CH₂CH₂SH)—CO—NH₂)—NH— |
| L5 | —CO—CH(CH₂CH₂SH)—NH—CO—CH₂—CH₂—CH(CO—NH—CH₂—CONH₂)—NH— |
| L6 | —CO—CH(CH₂CH₂COR¹)—NH—CO—CH₂—CH₂—CH(CO—NH—CH₂—CONH₂)—NH— |
| L7 | —CO—CH(CH₂COR¹)—NH—CO—CH₂—CH₂—CH(CO—NH—CH₂—CONH₂)—NH— |
| L8 | —CO—CH₂—NH—CO—CH₂—CH₂—CH(CO—NH—CH(CH₂CH₂COR¹)—CO—NH₂)—NH— |
| L9 | —CO—CH₂—NH—CO—CH₂—CH₂—CH(CO—NH—CH(CH₂COR¹)—CO—NH₂)—NH— |
| L10 | —CO—CH₂—NH—CO—CH₂—CH₂—CH(CO—NH—CH₂—COR¹)—NH— |
| L11 | —CO—CH(CH₂CH₂COOH)—NH—CO—CH₂—CH₂—CH(CO—NH—CH₂—CONH₂)—NH— |
| L12 | —CO—CH(CH₂COOH)—NH—CO—CH₂—CH₂—CH(CO—NH—CH₂—CONH₂)—NH— |
| L13 | —CO—CH₂—NH—CO—CH₂—CH₂—CH(CO—NH—CH(CH₂CH₂COOH)—CO—NH₂)—NH— |
| L14 | —CO—CH₂—NH—CO—CH₂—CH₂—CH(CO—NH—CH(CH₂COOH)—CO—NH₂)—NH— |
| L15 | —CO—CH₂—NH—CO—CH₂—CH₂—CH(CO—NH—CH₂—CO—NH—R¹)—NH— |

The R¹ groups in L6-L10 may be a weakly basic diamino group —NH—R²—NH₂. Preferred examples of R² are p-phenylene, o-phenylene or m-phenylene. Aniline is a simple and prototypic example of a weakly basic amine; the class of aromatic amines that are, in general, weakly basic. An aromatic amine is used to introduce an aromatic R¹ group. R¹ may be a homoaryl or a heteroaryl residue, and may be substituted with one or more substituents drawn from a broad range. The aromatic group may be polycyclic, wherein the various rings may be fused, unfused, or even both fused and unfused. In a polycyclic aromatic group, the rings may be homocyclic or heterocyclic, or even a mixture of both. The ring may be substituted with one or more substituents drawn from a broad range. For example, R¹ in L15 may be phenyl or substituted phenyl but need not be an aromatic residue for weak basicity.

Another class of suitable amines are those having the formula H₂N—CH₂—CO—NH—(CH₂)ₓ-homoaryl, or H₂N—CH₂—CO—NH—(CH₂)ₓ-heteroaryl, wherein x=2-10. The homoaryl or heteroaryl residue may be substituted with one or more substituents drawn from a broad range. As above, the homoaryl residue may be polycyclic, fused or unfused or both. The heteroaryl residue may additionally contain a homocyclic ring or more than one homocyclic rings that may be fused, unfused or even both fused and unfused. These compounds described above are non-limiting and are illustrative of the broad structural properties weakly basic amines within the scope of this invention.

Preparation of Cyclic Peptides

In the general formula, above, the amide bond (CO—NH) linking X¹ to X², is such that the carbonyl moiety is from amino acid X¹ and the amino moiety is from the amino acid X². The same is true for the link between X² and X³, and so on within the n-mer peptide. The peptide has X¹ as its N-terminus and Xⁿ as its C-terminus. To prepare a cyclic peptide 1, the linker is chosen to provide, at one terminus, a functional group that can be chemically bonded to the carboxyl C atom of amino acid Xⁿ and, at the other terminus, a functional group that can be chemically bonded to the α-amino N atom of amino acid X¹. Alternatively, the linear peptide can be synthesized with an extension at Xⁿ comprising a portion of the ultimate final linker group L; that extension is termed L_{b}. After synthesis of the peptide chain the X¹ terminus is extended with an extension that will also become part of the ultimate liner; this group is designated Lₐ. These steps yield a compound of the formula:

Lₐ-X¹-X²-X³-X⁴-X⁵-X⁶-X⁷-X⁸- . . . -Xⁿ-L_{b}.

The free ends of Lₐ and L_{b} are then chemically bonded to each other. In this way, the linker L is formed during the cyclization step from pre-attached fragments Lₐ and L_{b}. In the examples given below for L, the direction of L, reading left to right, is from to X¹ to X¹¹, i.e., the C-terminus of L is bonded to X¹, and the N-terminus of L is bonded to X¹¹.

When L includes a Cys, HomoCys, Glu, Asp, γ-carboxyl modified Glu or a β-carboxyl modified Asp residue, the configuration of the enantiomeric center of such a residue can be either L- or D-.

To prepare the compounds having a linker L of the L6, L7, L8, L9 or L10 type, the L is chosen to provide, at one terminus, a functional group that can be chemically bonded to the carboxyl C atom of amino acid Xⁿ and, at the other terminus, a functional group that can be chemically bonded to the α-amino N atom of amino acid X¹.

The R¹-group may be introduced into the linker L in two different ways (see below): (a) as part of the peptide synthesis on the resin, or; (b) by making a peptide intermediate with a linker L containing COOH in lieu of COR¹, which intermediate is subsequently modified to incorporate the R¹ group.

The above cyclic peptide compounds have the following properties: (a) high binding affinity to 447 (preferably 100 nM or less); (b) competitively inhibit the binding of 447 (or a fragment thereof) to V3_{MN}, gp120_{MN} or HIV-1_{MN} virions with an IC₅₀ value of less than about 10 μM, preferably less than about 1 μM, most preferably less than about 0.1 μM; (c) relatively weaker binding to another anti-V3 mAb which is poorly- or non-neutralizing.

### General Description of Peptidomimetics

A preferred type of chemical derivative of a V3 peptide described herein is a peptidomimetic compound which mimics the constrained V3 peptide and preferably improves certain biological actions of V3. A peptidomimetic agent may be an unnatural peptide or a non-peptide agent which recreates the stereospatial properties of the binding elements of a V3 peptide such that it has the binding activity or biological activity of the V3 peptide. Similar to a cyclic peptide based on a V3 sequence, a peptidomimetic will have a binding face (which interacts with 447 and/or with the R5 or X4 receptors) and a non-binding face. Again, similar to a cyclic peptide, the non-binding face of a peptidomimetic will comprise functional groups which can be modified by various therapeutic and diagnostic moieties without modifying the binding face of the peptidomimetic. One embodiment of a peptidomimetic would contain an aniline on the non-binding face. The NH₂-group of an aniline has a pKa˜4.5 and could therefore be modified by any amine-selective reagent without modifying any NH₂ functional groups on the binding face of the peptidomimetic. A peptidomimetics could lack NH₂ functional groups on its binding face so that any NH₂, without regard for pKₐ, could be displayed on the non-binding face as a site for conjugation. In addition other modifiable functional groups, such as —SH and —COOH could be incorporated into the non-binding face of a peptidomimetic as a site for conjugation.

This invention includes compounds which retain partial peptide characteristics. For example, any proteolytically unstable bond within the cyclic peptide could be selectively replaced by a non-peptidic element such as an isostere (N-methylation; substituted D-amino acid) or a reduced peptide bond while the rest of the molecule retains its peptide nature.

Various peptidomimetic compounds, including agonists, substrates and inhibitors, have been described for a number of bioactive peptides including opioid peptides, VIP, thrombin, HIV protease, etc. Methods for designing and preparing peptidomimetic compounds are known in the art (Huby, V. J., Biopolymers 33:1073-1082 (1993); Wiley, R. A. et al., Med. Res. Rev. 13:327-384 (1993); Moore et al., Adv. in Pharmacol 33:91-141 (1995); Giannis et al., Adv. in Drug Res. 29:1-78 (1997), which references are incorporated by reference in their entirety). These methods are used to make peptidomimetics that have the binding capacity and specificity of a 447-constrained V3 peptide and also have the desired biological activity described herein. Knowledge of peptide chemistry and general organic chemistry available to those skilled in the art are sufficient, in view of the present disclosure, for design and synthesis of such mimetic compounds.

For example, a peptidomimetics may be identified by inspection of the present NMR 3D structure of V3_{MN} or V3_{IIIB} bound to 447. Alternatively or additionally, the peptidomimetic may be based on X-ray crystallographically-derived 3D structure of the V3 peptide bound to 447 (or to an R5 or X4 receptor). The better knowledge of the stereochemistry of the interaction of the V3 ligand with 447 or with the chemokine receptor will assist in the rational design of such agents.

### Peptide Synthesis

The present peptides are synthesized by solid-phase methods well-known in the art. Solid-phase synthesis is generally described by Merrifield, J. Amer. Chem. Soc., 85:2149-54 (1963), although other equivalent chemical syntheses known in the art are also useful. For specific examples of methods used in the synthesis of mimics of CD4, see Vita, C et al., Proc. Natl. Acad. Sci. USA 92:6404-6408 (1995); Martin, L et al., Tetrahedron 56:9451-9460 (2000); Martin, L et al., Nature Biotechnol 21:71-76 (2003). Synthetic peptides are purified by reverse-phase HPLC and their identity verified by electrospray mass spectrometry.

Solid-phase peptide synthesis may be initiated from the C-terminus of the peptide by coupling a protected α-amino acid to a suitable resin. Such a starting material can be prepared by attaching an α-amino-protected amino acid by an ester linkage to a chloromethylated resin or to a hydroxymethyl resin, or by an amide bond to a BHA resin or MBHA resin. The preparation of the hydroxymethyl resin is described by Bodansky et al., Chem. Ind., 38:1597-1598 (1966). Chloromethylated resins are commercially available. The preparation of such a resin is described by Stewart et al. (Solid Phase Peptide Synthesis, Freeman & Co., San Francisco 1969, chapter 1, 1-6). BHA and MBHA resin supports are commercially available and are generally used only when the desired peptide being synthesized has an unsubstituted amide at the C-terminus. Coupling methods involving the use of a coupling agents such as N,N′dicyclohexylcarbodiimide or N,N′-diisopropylcarbodiimide and others are well-known in the art. See, for example, Gross et al., The Peptides: Analysis, Structure, Biology, Vol. I, Academic Press, 1979, the disclosure of which is hereby incorporated by reference.

The α-amino group of each amino acid employed in the peptide synthesis must be protected during the coupling reaction to prevent side reactions involving their active α-amino function. Certain amino acids have reactive side-chain functional groups (e.g., sulfhydryl, amino, carboxyl, and hydroxyl) that must also be protected with suitable protecting groups to prevent a chemical reaction from occurring during the initial and subsequent coupling steps. In selecting a particular protecting group, the following general rules are typically followed. An α-amino protecting group should render the α-amino function inert under the conditions of the coupling reaction, should be readily removable after the coupling reaction under conditions that do not remove side-chain protecting groups nor alter the structure of the peptide, and should substantially reduce the possibility of racemization upon activation, immediately prior to coupling.

Side-chain protecting groups should render the side chain functional group inert under the conditions of the coupling reaction, should be stable under the conditions employed to remove the α-amino protecting group, and should be readily removable from the fully-assembled peptide under conditions that do not alter the peptide chain's structure.

Conventional protecting groups include 2-(p-biphenyl)isopropyloxycarbonyl; t-butyloxycarbonyl (BOC), fluorenylmethyloxycarbonyl (FMOC), t-amyloxycarbonyl, adamantyl-oxycarbonyl, and p-methoxybenzyloxycarbonyl, benzyloxycarbonyl (CBZ), substituted CBZ, such as, e.g., p-chlorobenzyloxycarbonyl, p-nitrobenzyloxycarbonyl, p-bromobenzyloxycarbonyl, and p-methoxybenzyloxycarbonyl, o-chlorobenzyloxycarbonyl, 2,4-dichlorobenzyloxycarbonyl, 2,6-dichlorobenzyloxycarbonyl, and the like; cycloalkyloxycarbonyl, and isopropyloxycarbonyl. It is known that such groups vary in reactivity with the agents employed for their removal. See, for example, Gross et al., The Peptides: Analysis, Structure, Biology, Vol. 3, Academic Press, 1981 (incorporated by reference in its entirety). The preferred α-amino protecting groups are tBOC and FMOC. Other standard α-amino group de-protecting reagents, such as HCl in dioxane, and conditions for the removal of specific α-amino protecting groups are well-known in the art, e.g., Lübke et al., Chemie und Biochemie der Aminosaüren, Peptide und Proteine I, Chapter II-1, 102-117 (Georg Thieme Verlag Stuttgart 1975. incorporated by reference in its entirety).

An alternative to the stepwise approach is the fragment condensation method in which pre-formed peptides of shorter length, each representing part of the desired sequence, are coupled to a growing chain of amino acids bound to a solid phase support. For this stepwise approach, a particularly suitable coupling reagent is N,N′-dicyclohexyl-carbodiimide or diisopropylcarbodiimide. The selection of the coupling reagent, as well as the choice of the fragmentation pattern needed to couple fragments of the desired nature and size are important for success and are known to those skilled in the art.

In appropriate circumstances and when certain structural requirements of the peptide are met, when it is desired to cleave the peptide without removing protecting groups, the protected peptide-resin can be subjected to methanolysis, thus yielding a protected peptide with a methylated C-terminal carboxyl group. This methyl ester can be hydrolyzed under mild alkaline conditions to give the free carboxyl group. Protecting groups on the peptide chain can then be removed by treatment with a strong acid, such as liquid hydrogen fluoride. See, for example, Moore et al., In Peptides, Proc. Fifth Amer. Pept. Symp., 518-521 (Goodman et al., eds., 1977).

Purification of the cyclic peptides of the invention is typically achieved using chromatographic techniques, such as preparative HPLC including reverse phase TALC, or gel permeation, ion exchange, partition and/or affinity chromatography.

### General Molecular Structure Analysis, Modeling and Screening

Preferred software for use in processing and analysis of NMR spectra are XWINNMR, AURELIA, NMRVIEW and NMRDRAW. Structural calculation is preferably performed using CNS and CANDID (or their equivalents).

The present invention provides models of the 3D atomic structures of constrained V3 loop peptides. It will be understood by one of ordinary skill in the art that such models can be used to represent selected 3D structures and to perform comparative structure/function analyses of different peptides, or to design or identify molecule sharing such conformations.

The NMR coordinates of the structures of the present invention define the essential structure of the V3 loop as it binds to certain highly potent, broadly neutralizing anti-HIV-1 gp120 antibodies. This data define for the first time, certain novel conformations useful for designing new compounds for use as HIV-1 immunogens and anti-HIV-1 drugs. The structural “models” of the present invention have already provided new, significant insight into the relationship between HIV-1 V3 peptides and chemokines that bind to the same receptors. This information an be exploited in several ways that are described below. The structural information disclosed herein provides a unique and powerful tool enabling the rational design or identification of molecules for use in HIV-1 vaccines and drugs. Indeed, this invention provides methods for screening/identifying, as well as methods for designing and producing, peptides and peptidomimetics with newly described an useful conformation for serving as HIV-1 immunogens and inhibitors.

Various methods of computationally screening compounds capable of specifically binding to a set of atoms whose atomic positioning and structure is modeled by the NMR coordinates of the present invention are well known in the art. See, for example, Bugg et al, (1993). Sci. Amer. (December), pg. 92; West et al. (1995) TIPS 16:67; Dunbrack et al (1997) Fold. Des. 2:R27-42).

For example, potential mimics of the V3 loop structures that bind to the 447 binding pocket and/or to R5 or X4 receptors can be examined through the use of computer modeling using docking programs such as GRAM, DOCK, or AUTODOCK (Dunbrack et al., supra). Use Of such programs permit predicting or calculating the orientation, binding constant or relative affinity of a given compound to a structure and the use of that information to design or select compounds of the desired affinity. A database or library of chemical structures is searched and computational fitting of compounds is performed to identify those molecules with one or more functional groups suitable for the desired interactions. With these methods, one can ascertain how effectively candidate compounds mimic the binding of a constrained V3 loop peptide to an antibody or a receptor.

Molecular docking programs may also be effectively used in conjunction with structure modeling programs (see below). Using computational approaches, compounds can furthermore be systematically modified by molecular modeling programs until promising molecular structures are achieved. This technique has proved effective, for example, in the development of HIV protease inhibitors (Wlodawer et al. (1993). Ann Rev Biochem. 62:543; Appelt (1993) Persp Drug Discov Design 1:23; Erickson (1993) Persp Drug Discov Design 1:109). Thus, the use of computational screening enables larger numbers of compounds to be rapidly screened and produces small numbers of putative hits without the requirement of resorting to the laborious synthesis of large numbers of compounds. Once putative mimics are computationally identified they can either synthesized de novo. Candidate molecules are tested for their ability to bind to broadly neutralizing anti-V3 loop antibodies such as 447, or to chemokine receptors, using any conventional direct or competitive binding assay. Alternatively or additionally, candidate compounds are functionally qualified, for example, via testing of their ability to inhibit virus infection in-vitro or in vivo in an animal model. When suitable molecules are identified (or designed), further NMR structural analysis can optionally be performed on them in binding complexes as has been done here in Example X (and Tables 5 and 6 for the new X4-M1 and R5A-M1 peptides designed according to the methods set forth herein. peptides. Promising peptides can be readily and economically synthesized in large quantities for clinical use, since such production highly automated and quality is easy to control. (See, for example, Patarroyo, M (1990). Vaccine 10:175).

Solid phase-based assays for screening binding (to antibody or receptor) are well known in the art. Another effective way to test binding interactions is via surface plasmon resonance (SPR) analysis, using, for example, commercially available BIAcore chips (Pharmacia). Such chips are coated with either the peptide or an antibody or receptor or fragment thereof, and changes in surface conductivity measured as a function of binding affinity upon exposure of one member of the putative binding pair to the other member.

Models of the structure of the constrained peptides or mimetics of the present invention can be utilized, respectively, to facilitate solution of the 3D structures. This may be done computationally via-molecular replacement, where all or part of a model of a constrained peptide is used to determine the structure of a crystallized macromolecule or macromolecular complex having a closely related but unknown structure. Solution of an unknown structure by molecular replacement involves obtaining X-ray diffraction data for crystals of the macromolecule or macromolecular complex for which one wishes to determine the 3D structure. The 3D structure of a macromolecule or macromolecular complex whose structure is unknown is obtained by analyzing X-ray diffraction data derived therefrom using molecular replacement techniques with reference to the structural coordinates of the present invention as a starting point to model the structure thereof (See, for example, U.S. Pat. No. 5,353,236). The molecular replacement technique is based on the principle that two macromolecules which have similar structures, orientations and positions in the unit cell diffract similarly. Molecular replacement involves positioning the known structure in the unit cell in the same location and orientation as the unknown structure. Once positioned, the atoms of the known structure in the unit cell are used to calculate the structure factors that would result from a hypothetical diffraction experiment. This involves rotating the known structure in the six dimensions (three angular and three spatial dimensions) until alignment of the known structure with the experimental data is reached. This approximate structure can be fine-tuned to yield. a more accurate and often higher resolution structure using various refinement techniques.

The structure models of the present invention may be generated by a computing platform which generates a graphic output of the models via a display. The computing platform generates graphic representations of atomic structure models via a processing unit which processes structure coordinate data stored in a retrievable format in data storage device. Examples of computer readable media which can be used to store coordinate data include conventional computer hard drives, floppy disks, DAT tape, CD-ROM, and other magnetic, magneto-optical, optical, floptical, and other media which may be adapted for use with computing platform. See for example, PCT Publication WO03/026562. Suitable software applications known to those of skill in the art, which may be used by processing unit to process structure coordinate data so as to provide a graphic output of 3D structure models include: ICM-Pro (Molsoft, LLC, WWW address: molsoft.com), INSIGHT, MOLMOL, RASMOL, QUANTA, CHARMM, SYBYL (WWW address: tripos.com/softward/sybase.html), MACROMODE, GRASP, RIBBONS (Carson, M (1997) Meth Enzymol 277:25; Jones, T A et al. (1991) Acta Crystallogr 47:110), DINO (DINO: Visualizing Structural Biology (2001) WWW site: dino3d.org). Some of these are reviewed in Kraulis, J (1991) Appl Crystallogr. 24:946).

The structure coordinates of the present invention as shown herein are slightly modified from the standard PDB format. The standard PDB format is preferred for convenient processing by various of these software applications. Most or all of these software applications as well as others may be obtained by download from the World Wide Web.

Other useful programs for the present invention include: SCULPT (helps in energy minimization and amino acid manipulation of models by generating low-energy 3D confirmations; WWW address: mdli.com/cgi/dynamic/product.html0; MODELLAR (conducts homology modeling of sequence alignments using satisfaction of spatial restraints when calculating a protein structure; Web address: guitar.rockefeller.edu/modeller/modeller.htm) PredictProtein (accepts an amino acid sequence and returns a secondary structure prediction; WWW address cubic.bioc.columbia.edu/predictprotein/

### Pharmaceutical and Therapeutic Compositions and Methods

The peptides and other mimetic compounds of the present invention are well suited for the preparation of pharmaceutical compositions. The pharmaceutical compositions may be administered to any animal which may experience the beneficial effects of the composition. Foremost among such animals are humans, although the invention is not intended to be so limited.

Thus, the present invention provides a method for treating a subject in need of treatment with a conformationally constrained V3 loop peptide or other mimic as described herein. Using methods described herein, or other methods well-known in the art for establishing biological activity of the peptide or mimic, one or ordinary skill in the art will be able to determine without undue experimentation the relevant biological activity of a peptide, analogue, isostere or other mimetic according to the present invention. A composition of this invention may be active per se, or may act as a “pro-drug” that is converted in vivo to the active form, e.g., proteolytic cleavage.

To determine the activity of the compound an immunogen, one generally measures the antibody response of the recipient by obtaining a serum sample at appropriate intervals in the immunization schedule and testing it for antibodies that (a) bind a V3 peptide, gp120, HIV-1 virions or infected cells, and (2) neutralize the virus. Binding assays for anti-HIV-1 antibodies are conventional and are described in detail in many of the references cited herein. HIV-1 neutralization assays are also well known in the art, and exemplary description may be found in Mascola J R et al. (2002) J. Virol. 76:4810-21; Montefiori D C et al. (1988) J Clin Microbiol 26:231-235; and D'Souza M P et al. (1997) J. Infec. Dis. 175:1056-62. The ideal approach for expressing neutralization potency of an antiserum or purified antibody are still unsettled in the art. Art-recognized values include % neutralization compared to a control, titer (dilution of the serum that yields positive neutralization, the concentration of a purified antibody that results in neutralization, or a statistically significant neutralization such as exceeding 5% confidence limits of a negative control.

To determine the activity of the present compound as an antagonist of viral binding to co-receptors or of post-binding infectious events, the compound is tested in a standard assay of binding to a purified R5 or X4 receptor or to a cell expressing such receptors. The compound is titered against a fixed amount of a labeled ligand, for example, and the IC₅₀ (concentration that gives half maximal inhibition) is calculated. The compound can be tested for induction of receptor internalization (or desensitization) by exposing receptor-bearing cells to the compound and testing at various intervals for the cells' ability to bind a known ligand.

A pharmaceutical composition comprising the constrained peptide or other mimic may then be administered to a subject, preferably a human, having, or at risk for, a disease or condition that benefits from such treatment, primarily HIV-1 infection or HIV-1 disease/AIDS.

The term “treating” includes administering a pharmaceutical or immunogenic composition as above to prevent, ameliorate, inhibit the progression or, or cure the disease or condition. Such treating may be performed alone or in conjunction with other therapies.

The present invention thus includes a “pharmaceutical” or “immunogenic” composition comprising the V3 peptide, derivative, analogue, isostere or mimetic along with a pharmaceutically or immunologically acceptable excipient. Thus, the term “therapeutic composition” includes immunogenic or vaccine compositions and any other pharmaceutical comprising the V3 peptide/mimic and a therapeutically acceptable carrier or excipient. General methods to prepare immunogenic or vaccine compositions are described in Remington's Pharmaceutical Science; Mack Publishing Company Easton, Pa. (latest edition).

The invention provides a method of treating a subject, preferably a human, by immunizing or vaccinating the subject to induce a neutralizing antibody response and any other accompanying protective form of immune reactivity. Also provided is a method for inhibiting viral infection or spread of virus by exploiting the co-receptor specificity of the V3 constrained peptide or mimic.

The immunogenic material may be adsorbed to or conjugated to beads such as latex or gold beads, ISCOMs, and the like. Immunogenic compositions may comprise adjuvants, which are substance that can be added to an immunogen or to a vaccine formulation to enhance the immune-stimulating properties of the immunogenic moiety. Liposomes are also considered to be adjuvants (Gregoriades, G. et al., Immunological Adjuvants and Vaccines, Plenum Press, New York, 1989) Examples of adjuvants or agents that may add to the effectiveness of proteinaceous immunogens include aluminum hydroxide, aluminum phosphate, aluminum potassium sulfate (alum), beryllium sulfate, silica, kaolin, carbon, water-in-oil emulsions, and oil-in-water emulsions. A preferred type of adjuvant is muramyl dipeptide (MDP) and various MDP derivatives and formulations, e.g., N-acetyl-D-glucosaminyl-(β1-4)-N-acetylmuramyl-L-alanyl-D-isoglutamine (GMDP) (Hornung, R L et al. Ther Immunol 1995 2:7-14) or ISAF-1 (5% squalene, 2.5% pluronic L121, 0.2% Tween 80 in phosphate-buffered solution with 0.4 mg of threonyl-muramyl dipeptide; see Kwak, L W et al. (1992) N. Engl. J. Med., 327:1209-1238). Other useful adjuvants are, or are based on, bacterial endotoxin, lipid X, whole organisms or subcellular fractions of the bacteria Propionobacterium acnes or Bordetella pertussis, polyribonucleotides, sodium alginate, lanolin, lysolecithin, vitamin A, saponin and saponin derivatives such as QS21 (White, A. C. et al. (1991) Adv. Exp. Med. Biol., 303:207-210) which is now in use in the clinic (Helling, F et al. (1995) Cancer Res., 55:2783-2788; Davis, T A et al. (1997) Blood, 90: 509), levamisole, DEAE-dextran, blocked copolymers or other synthetic adjuvants. A number of adjuvants are available commercially from various sources, for example, Merck Adjuvant 65 (Merck and Company, Inc., Rahway, N.J.) or Freund's Incomplete Adjuvant and Complete Adjuvant (Difco Laboratories, Detroit, Mich.), Amphigen (oil-in-water), Alhydrogel (aluminum hydroxide), or a mixture of Amphigen and Alhydrogel. Aluminum is approved for human use.

An additional therapeutic compositions and methods comprise antibodies or an antiserum induced in one subject using the present immunogen, removed from that subject and used to treat another subject by passive immunization or transfer of the antibodies. This is particularly useful for treating neonates exposed to maternal virus, healthcare workers immediately after acute exposure to HIV-1 through patient contact or material handling, or shortly after primary exposure to HIV-1 through sexual contact. For disclosure of such passive immunization with patient sera, neutralizing antisera or mAbs, see Nishimura Y et al. (2003) Proc Natl Acad Sci USA 100:15131-36; Mascola J R (2003) Curr Mol Med. 3:209-16; Ferrantelli F et al. (2003) AIDS 17:301-9; Ferrantelli F et al (2002) Curr Opin Immunol. 14:495-502; Xu W et al. (2002) Vaccine 20:1956-60; Nichols C N et al. (2002) AIDS Res Hum Retrovir. 8:49-56; Cho M W et al. (2000) J. Virol. 74:9749-54; Mascola J R et al. (2000) Nat Med. 6:207-10; Andrus. L et al. (1998) J. Inf. Dis. 77: 889-897; Parren P W (1995) AIDS 9:F1-6; Hinkula J et al. (1994) J Acquir Immune Defic Syndr. 7:940-51; Prince A M et al. (1991) AIDS Res Hum Retrovir 7:971-73; Emini E A et al. (1990) J. Virol. 64:3674-84, all incorporated by reference.

The amount of active compound to be administered depends on the precise peptide or mimic selected, the health and weight of the recipient, the route of administration, the existence of other concurrent treatment, if any, the frequency of treatment, the nature of the effect desired, and the judgment of the skilled practitioner.

A preferred effective dose for treating a subject in need of the present treatment, preferably a human, is an amount of up to about 100 milligrams of active compound per kilogram of body weight. A typical single dosage of the peptide, chimeric protein or peptidomimetic is between about 1 ng and about 100 mg/kg body weight, and preferably from about 10 μg to about 50 mg/kg body weight. A total daily dosage in the range of about 0.1 milligrams to about 7 grams is preferred for intravenous administration. A useful dose of an antibody for passive immunization is between 10-100 mg/kg. It has been suggested (see references cited above for passive immunity) that an effective in vivo dose of an antibody/antiserum is between about 10- and 100-fold more than an effective neutralizing concentration or dose in vitro. These dosages can be determined empirically in conjunction with the present disclosure and state-of-the-art.

The foregoing ranges are, however, suggestive, as the number of variables in an individual treatment regime is large, and considerable excursions from these preferred values are expected. As is evident to those skilled in the art, the dosage of an immunogenic composition may be higher than the dosage of the compound used to treat infection (i.e., limit viral spread). Not only the effective dose but also the effective frequency of administration is determined by the intended use, and can be established by those of skill without undue experimentation. The total dose required for each treatment may be administered by multiple doses or in a single dose. The peptide or mimetic may be administered alone or in conjunction with other therapeutics directed to the treatment of the disease or condition.

Pharmaceutically acceptable acid addition salts of certain compounds of the invention containing a basic group are formed where appropriate with strong or moderately strong, non-toxic, organic or inorganic acids by methods known to the art. Exemplary of the acid addition salts that are included in this invention are maleate, fumarate, lactate, oxalate, methanesulfonate, ethanesulfonate, benzenesulfonate, tartrate, citrate, hydrochloride, hydrobromide, sulfate, phosphate and nitrate salts. Pharmaceutically acceptable base addition salts of compounds of the invention containing an acidic group are prepared by known methods from organic and inorganic bases and include, for example, nontoxic alkali metal and alkaline earth bases, such as calcium, sodium, potassium and ammonium hydroxide; and nontoxic organic bases such as triethylamine, butylamine, piperazine, and tri(hydroxymethyl)methylamine.

The compounds of the invention, as well as the pharmaceutically acceptable salts thereof, may be incorporated into convenient dosage forms, such as capsules, impregnated wafers, tablets or preferably injectable preparations. Solid or liquid pharmaceutically acceptable carriers may be employed.

Preferably, the compounds of the invention are administered systemically, e.g., by injection or infusion. Administration may be by any known route, preferably intravenous, subcutaneous, intramuscular, intrathecal, intracerebroventricular, or intraperitoneal. (Other routes are noted below) Injectables can be prepared in conventional forms, either as solutions or suspensions, solid forms suitable for solution or suspension in liquid prior to injection, or as emulsions.

To enhance delivery or immunogenic activity, the compound can be incorporated into liposomes using methods and compounds known in the art.

The pharmaceutical preparations are made following conventional techniques of pharmaceutical chemistry. The pharmaceutical compositions may also contain minor amounts of nontoxic auxiliary substances such as wetting or emulsifying agents, pH buffering agents and so forth. The peptides are formulated using conventional pharmaceutically acceptable parenteral vehicles for administration by injection. These vehicles are nontoxic and therapeutic, and a number of formulations are set forth in Remington's Pharmaceutical Sciences, Gennaro, 18th ed., Mack Publishing Co., Easton, Pa. (1990)). Nonlimiting examples of excipients are water, saline, Ringer's solution, dextrose solution and Hank's balanced salt solution. Formulations according to the invention may also contain minor amounts of additives such as substances that maintain isotonicity, physiological pH, and stability. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides. Aqueous injection suspensions may contain substances which increase the viscosity of the suspension. Optionally, a suspension may contain stabilizers.

The peptides and other useful compositions of the invention are preferably formulated in purified form substantially free of aggregates and other protein materials, preferably at concentrations of about 1.0 ng/ml to 100 mg/ml.

As noted above, therapeutic compositions of the invention may comprise, in addition to the peptides, analogues, isosteres, mimics, chimeric proteins or cyclic peptides, one or more additional anti-HIV agents, such as protease inhibitors or reverse transcriptase inhibitors as well as immunostimulatory agents including cytokines such as interferons or interleukins. In fact, pharmaceutical compositions comprising any known HIV therapeutic in combination with the compounds disclosed herein are within the scope of this invention. The pharmaceutical composition may also comprise one or more other medicaments to treat additional symptoms for which the target patients are at risk, for example, anti-infectives including antibacterial, anti-fungal, anti-parasitic, anti-viral, and anti-coccidial agents.

### Additional Uses of the Constrained Peptides or Peptidomimetics

An additional use for the present compounds is as an affinity ligand for isolating or enriching or selecting:
(a) neutralizing antibodies of the desired fine specificity;
(b) B cells expressing on their surface immunoglobulins a V region with the specificity of the above antibodies.
(c) R5, X4 or other chemokine receptors; or
(d) cells expressing R5, X4 or other chemokine receptors.
   For such uses, the peptide, preferably part of a fusion protein, is immobilized to any solid support known in the art.

Having now generally described the invention, the same will be more readily understood through reference to the following examples which are provided by way of illustration, and are not intended to be limiting of the present invention, unless specified.

### Example I

### Experimental Procedures and Materials

Note on letter/number codes: The MN or IIIB superscript preceding the single letter amino acid code indicates the HIV_{MN} or HIV_{IIIB} strain origin of the sequence; the number following the amino acid code represents the position of the residue in the full length gp120_{MN} or gp120_{IIIB} sequence. The number is sometimes followed by the position of the hydrogen (H) involved in the hydrogen bonding—i.e., an amino hydrogen (H^{N}) or a hydrogen atom bonded to the α carbon (H^{α})

### Sample Preparation

The V3_{MN} peptide, ³⁰⁸⁻³³²gp120_{MN} (YNKRKRIHI--GPGRAFYTTKNIIG; SEQ ID NO:13) linked to a fusion protein was expressed in E. coli, cleaved and purified as previously described by M. Sharon et al. (2002) Protein Expr. Purif. 24:374-383.). Note that the sequential numbering system in V3_{MN} is interrupted due to a rare two residue insertion in HIV-1_{IIIB} and therefore residues 317 and 318 are not present in V3_{MN}. The 447Fv was expressed in BL21(DE3)pLysS strain.

The Fv-peptide complex (28.7 kDa) was prepared by the addition of a 20% molar excess of the peptide to a dilute solution of the Fv fragment (˜0.04 mM). The sample was concentrated by membrane filtration using Vivaspin (Vivascience) with a 10 kDa cut-off. All samples contained 10 mM sodium phosphate buffer and 0.05% NaN₃.

### Preparation of V3_{IIIB} Peptide

The V3_{IIIB} peptide ³¹⁰⁻³²⁹gp120_{IIIB} (TRKSIRIQRGPGRAFVTIGK; SEQ ID NO:37) linked to a fusion protein was expressed in Escherchia coli, cleaved and purified as described by Sharon et al. (2002) supra Protein Expr Purif. 24:374-383. However, since Thr residue follows the Met, the efficiency of the cleavage in 70% formic acid was very low (Kaiser, R et al. (1999) Anal Biochem. 266:1-8). Therefore the cleavage was performed in 70% TFA. The 447-52D Fv was expressed in the BL21(DE3)pLysS strain as described by Kessler, N et al. (2003) Protein Expr Purif. 29:291-303). The Fv-peptide complex (28.3 kDa) was prepared by the addition of a 20% molar excess of the peptide to a dilute Fv solution (˜0.04 mM). The sample was concentrated by membrane filtration with vivaspin (Vivascience) with a 110 kDa cut-off. All samples contained 10 mM D-acetic acid buffer and 0.05% NaN₃ (pH 5).

### Preparation of Disulfide-Constrained R5A-M1 Peptide

All the Cys-containing peptides were synthesized by solid phase methods following the Fmoc strategy and purified with analytical and preparative HPLC systems using gradients from water/acetonitrile in 0.1% TFA. Final products were characterized by mass spectrometry and amino acid analysis.

The structure of this compound is set forth in Example X. For the selective formation of the two disulfide bonds, a combination of trityl (Trt) and acetamidomethyl (Acm) was chosen: Cys-1 and Cys-18 were protected with Trt. Cys-7 and Cys 12 were protected with Acm. The Trt group is labile to TFA and was consequently removed during the normal course of the cleavage reaction. Acm is stable to the conditions required for the cleavage and removal of all other protecting groups. The first disulfide bond was formed after selective removal of Trt by air oxidation; generation of the second disulfide bond was then carried out in a single step by treatment of the Acm-protected peptide with iodine, using aqueous AcOH as solvent to limit iodination of Tyr and His.

### Preparation of Disulfide-Constrained X4-M1 Peptide

The structure of this compound is set forth in Example X. The disulfide bond formation was controlled just as above using Trt and Acm, with the difference only in the positions of the Cys residues. Thus, Cys-2 and Cys-17 were protected with Trt and Cys-6 and Cys-13 with Acm. Removal of the protective groups was as above.

### NMR Spectroscopic Analysis of Peptides

### V3_{MN} and V3_{IIIB}

NMR spectra were acquired at 35° C. on a Bruker DMX 500 and DRX 800 spectrometer using unlabeled ³⁰⁸⁻³³²gp120_{MN} or peptide uniformly labeled with ¹⁵N, or with ¹³C and ¹⁵N in complex with unlabeled 447Fv. ROESY and HOHAHA spectra with long mixing times (90 ms) were used for epitope mapping. The mixing time was adjusted to discriminate between cross peaks of peptide protons immobilized in the complex due to interactions with the antibody and have a short T_{1ρ} relaxation time and those of protons that do not interact with the Fv and therefore retain considerable mobility and have a long T_{1ρ}. 2D spectra of the unlabeled complex were measured at 30, 20 and 10° C. and at pH values of 7, 5 and 4.25. The combination of the HOHAHA and ROESY spectra was used for sequential assignment of the mobile segments of the peptide in the Fv/peptide complex. A 2D ¹⁵N-edited TOCSY of 15N labeled peptide in complex with unlabeled Fv was measured to confirm the definition of the epitope. T₂ 15N relaxation time measurements (Kay, L E et al. (1992) J. Mag. Res. 97:359-375) were carried out using a total of 182 transients. Six time points were collected using parametric delays of 8, 16, 24, 32, 48, and 72 ms at 18.79 T with a 2s delay between scans.

Complete sequential and sidechain assignment of ¹H, ¹³C and ¹⁵N resonances of the bound peptide, including the epitope residues, was accomplished using TROSY-HNCA, CT-CBCA(CO)NH, TROSY-HNCACB, HBHA(CO)NH, HCCH-COSY and HCCH-TOCSY experiments (Sattler, M et al. (1999) Prog. Nuc. Mag. Res. Spec. 34:93-158 and references therein). The assignment of the aromatic sidechains was done using 2D ¹³C-TROSY (Pervushin, K (2000). Q. Rev. Biophys. 33:161-197 and references therein).

Distance constraints were derived from two 3D ¹³C-edited NOESY spectra, one optimized for the aliphatic protons and the other for the aromatic (80 ms mixing time in D₂O) and a ¹⁵N-edited TROSY-NOESY in H₂O (75 ms mixing time). Slowly exchanging amide protons were identified by recording a series of 2D ¹⁵N TROSY-HSQC spectra immediately after the H₂O buffer was exchanged with D₂O buffer. The 3D ¹⁵N- and ¹³C-separated NOESY spectra acquired using a ¹³C/¹⁵N-labeled V3_{MN} peptide, ³⁰⁸⁻³³²gp120_{MN}, in complex with the unlabeled 447Fv, revealed inter- and intra-molecular peptide NOEs.

Spectra were similarly acquired using ¹³C/¹⁵N-labeled V3_{IIIB} peptide, ³¹⁰⁻³²⁹gp120_{IIIB} bound to 447-52D Fv.

### Disulfide-Constrained Peptides

Two disulfide bond-constrained peptides were produced and analyzed. One designed to mimic 441-constrained V3_{MN} peptide had the sequence ³¹⁰CRKSIHC--GPGRCFYTTGC³²⁹ [SEQ ID NO:18]. The residue numbering of this 18-mer is based on the gp120 residue numbering used for “native” V3 peptides. This peptide is designated R5A-M1.

A second peptide was designed to mimic the X4 conformation (e.g., V3_{IIIB} conformation that is recognized and constrained by the mAb 0.5β. This peptide had the sequence ³¹⁰GCKSICI--GPGRACYTTCG³²⁹ [SEQ ID NO:19] and was designated X4-M1

### Analysis and Structure Calculations

φ-angle restraints were determined from ³J_{HNHα} coupling constants obtained from a 3D HNHA spectrum (Vuister, G W et al. (1993) JACS 115, 7772-77). The values of ³J_{HNHα} determined from peak intensity ratio were scaled by a factor of 1.2 to account for fast spin-flips during the dephasing period. The φ angles of residues with ³J_{HNHα} smaller than 6 Hz and larger than 8.5 Hz were constrained to −65°±25° and −120°±30° respectively. ³J_{HNHα} values between 6 and 8 Hz were considered uninformative (Roberts, G C K (1993) NMR of macromolecules (New York, Oxford University Press). Three ψ angles for residues ^{MN}I314, ^{MN}H315 and ^{MN}I316, were included in the calculations based upon analyses of predictions from the TALOS program (Cornilescu, G. et al. (1999) J. Biomol. NMR 13:289-302) using chemical shifts of ¹H, ¹³Cα ¹³Cβ and ¹⁵N. N spectra were processed with NMRpipe/NMRDraw (Delaglio, F et al. (1995) J. Biomol. NMR 6:277-293) or with Bruker's XWINNMR software and analyzed using AURELIA (Neidig, K-P et al. (1995) J. Biomol. NMR 6:255-270).

Interproton distance restraints were obtained from peak intensities. 0.5 Å was added for each NOE involving a methyl group, and 1 Å for constraints involving methyl-methyl interaction. The upper bound distance constraints were 130% of the NOE derived distances to account for internal motion and proton multiplicity (Roberts, supra) and the lower bound distance was set to 1.8 Å. Structure calculations were performed using CNS 1.1 (Brunger A T et al (1998) Acta. Crystallogr. D Biol. Crystallogr. 54:905-921) and a dynamic simulated-annealing protocol starting with extended initial structures. The ambiguous NOEs were assigned in an iterative manner using structures calculated based on the already assigned NOEs. Two hydrogen bonds were used as restraints in later stages of refinement on the basis of characteristic backbone NOEs between two anti-parallel β-strands. Secondary structure elements and rmsd values were calculated with the MOLMOL program 2.6 (Koradi, R et al. (1996) J. Mol. Graph. 14:51-55, 29-32). Structures were further analyzed with Aqua/Procheck-NMR (Laskowski, R A et al. (1996) J. Biomol. NMR 8:477-486) and displayed with InsightII (MSI Crop., US).

### Example II

### Mapping the V3_{MN} Epitope

NMR dynamic filtering was used to map the epitope within the V3 peptide recognized by the 447Fv. Peptide protons that do not interact with the Fv retain considerable mobility in comparison to peptide protons which do interact. As a result of the long mixing period used in the HOHAHA and ROESY spectra, the cross peaks of peptide protons interacting with the Fv as well as of most Fv protons vanish while the cross peaks of residues in the flexible parts of the peptide that do not interact with the Fv continue to be observed. These include seven residues of the C-terminal region (^{MN}T326-^{MN}G332) and two of the N-terminal segment (^{MN}N309, ^{MN}R311). The proton chemical shifts of these residues were practically identical to those observed for the free peptide, confirming that they do not interact, or have only very minor interactions with the antibody. The HOHAHA cross-peaks of ^{MN}K312-^{MN}R322 were undetectable in the spectra, implying strong interactions with 447Fv. The cross-peaks of ^{MN}A323, ^{MN}F324 and ^{MN}Y325 were weak, indicating that these three residues are part of the V3_{MN} epitope.

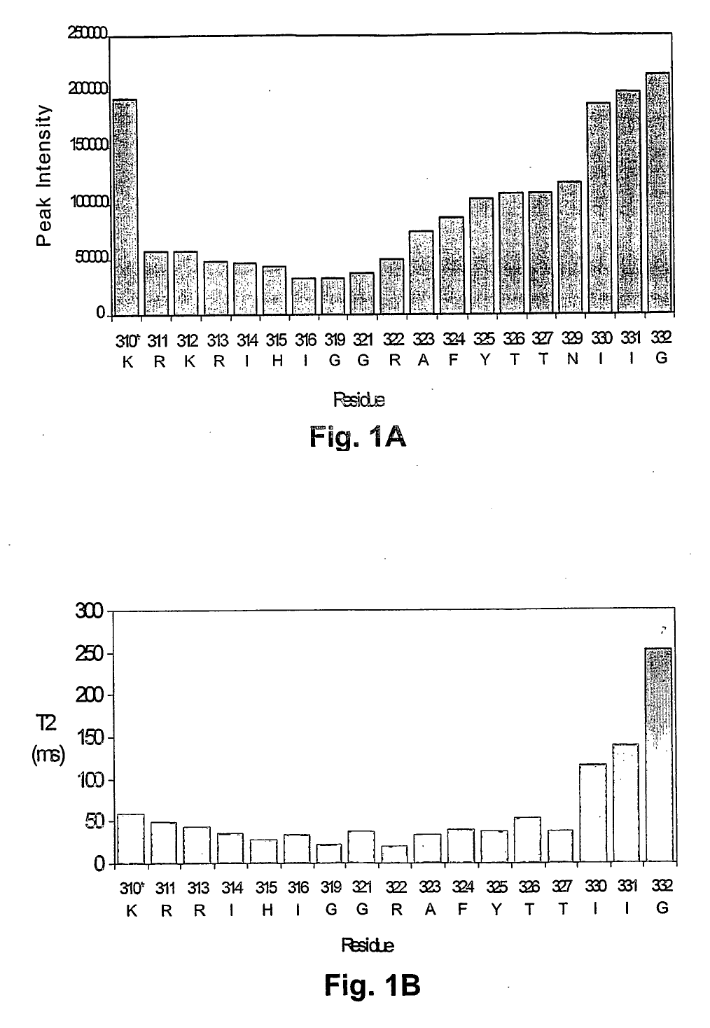

Using this method, the epitope recognized by the 447Fv was mapped to gp120 residues ^{MN}K312-^{MN}Y325. This definition of the epitope was confirmed by examining the peak intensity in a TROSY ¹H-¹⁵N HSQC spectrum (FIG. 1A) and by measurement of a HOHAHA spectrum of a ¹⁵N-labeled peptide in complex with unlabeled Fv which eliminated all interference by the Fv resonances (data not shown). Short ¹⁵N(¹H) T₂ relaxation times were measured for ^{MN}R311-^{MN}K328, indicating that some backbone immobilization of four additional residues, ^{MN}R311, ^{MN}T326, ^{MN}T327 and ^{MN}K328 (FIG. 1B) occurred due to their proximity to the epitope and minor interactions with the 447Fv (see below).

### Example III

### Solution Structure of the Antibody-Bound V3_{MN} Peptide

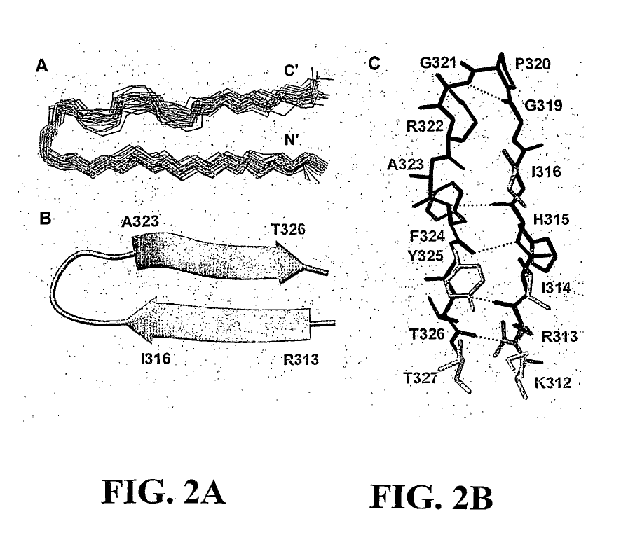

The structure of the bound V3_{MN} epitope was determined using 305 NMR-derived distance (90 long and medium range), 10 dihedral angle and 2 hydrogen bonds constraints. The superposition of the 29 lowest energy structures that satisfied the experimental restraints with no NOE violations larger than 0.5 Å and no torsion angle violations exceeding 5° is shown in FIG. 2A. The overall structure of the epitope (³¹²⁻³²⁷gp120) is well defined with root-mean-square deviations (rmsd) values of 0.37 Å and 1.17 Å for the backbone and heavy atoms, respectively. The structural statistics and rmsd are presented in Table 1. A Ramachandran plot (not shown) of the mean structure of the complex suggests that the φ and ψ angles of the structure predominantly occupy allowed regions except for ^{MN}G319 and ^{MN}G321.

The average NMR coordinates for the V3 MN peptide as bound by and constrained by the 447Fv antibody fragment are shown in Table 3. The individual values for the 29 lowest energy structures are publicly available, deposited in the Protein Database (PDB) under PDB-ID 1NJ0. The information in that file is hereby incorporated by reference in its entirety.
In Tables 3-6, the following abbreviations are used for some of the atoms: N—backbone nitrogen; HN—hydrogen bonded to this nitrogen; Cα—α carbon; Hα—hydrogen bonded to α carbon; Cβ—β carbon; Hβ—hydrogen bonded to βα carbon; Oγ1—γ-oxygen; Hγ1—γ1 hydrogen; Cγ2—γ2 carbon; Cδ1—δ1 carbon; Hδ1—hydrogen bonded to δ1 carbon; Cδ2—δ2 carbon, etc.

As shown in FIGS. 2A-2C, the epitope forms a β-hairpin consisting of two antiparallel β-strands formed by residues ^{MN}R313-^{MN}I316 and ^{MN}A323-^{MN}T326, linked by a reverse γ turn. NOE interactions characteristic of a β-hairpin conformation were observed between backbone atoms of the N-terminal “half” molecule and the C-terminal “half” molecule. These interactions include the following:

^{MN}R313 H^{N}-^{MN}T326 H^{N},

^{MN}R313 H^{N}-^{MN}T327 H^{α},

^{MN}I314 H^{α}-^{MN}Y325 H^{α} and

^{MN}H315 H^{N}-^{MN}Y325 H^{α}.

The expected NOE interactions between ^{MN}K312 H^{α}/^{MN}T327 H^{α} and between ^{MN}I314 H^{α}/^{MN}T326 H^{N} could not be assigned due to resonance overlap. ³J_{HNHα} coupling constants higher than 8.4 Hz, typical of a β-strand, were measured for ^{MN}I314, ^{MN}H315, ^{MN}I316, ^{MN}Y325, ^{MN}T326 and ^{MN}T327.

In the NMR structure of the V3 epitope (³¹²⁻³²⁷gp120), the β-hairpin is stabilized by a network of hydrogen bonds between the two β-strands (FIG. 2C). Two pairs of hydrogen bonds are formed between ^{MN}R313 and ^{MN}T326 and between ^{MN}H315 and ^{MN}F324. The side chains of residues ^{MN}R313, ^{MN}H315, ^{MN}F324 and ^{MN}T326 form the lower face of the β-hairpin while the side chains of ^{MN}I314, ^{MN}I316, ^{MN}A323 and ^{MN}Y325 form the upper face (FIG. 2C). The structure of the β-hairpin is stabilized by extensive hydrophobic interactions involving ^{MN}I314, ^{MN}I316 and ^{MN}Y325. The side chain of ^{MN}K312 forms additional stabilizing interactions with ^{MN}T327, ^{MN}I314 and ^{MN}Y325.

On the lower face of the β-sheet, only interactions between ^{MN}F324 and ^{MN}T326 could be observed, indicating that the lower face is less compact than the upper face. The precision and accuracy of the conformation of the side chains is expected to be improved when the structure of the entire V3 peptide-Fv complex is solved. However, due to side chain interactions within the peptide, the conformation of some of the side chains is very well defined in the structure of the bound V3MN peptide. For example, the heavy atom rmsd for ^{MN}I314 and ^{MN}I316 is 0.175 and 0.361 Å, respectively, for the best backbone superposition of residues ^{MN}K312-^{MN}T327.

### Example IV

### The GPG Segment Bound to 447Fv Forms an Inverse γ-Turn

The GPG sequence linking the β-strands forms an inverse γ-turn stabilized by an i,i+2 hydrogen bond between the carbonyl oxygen of ^{MN}G319 and the amide proton of ^{MN}G321. This γ-turn conformation is corroborated by the sequential H^{δ}-H^{α} and H^{δ}-H^{N} connectivities between ^{MN}P320 and ^{MN}G319 (typical of Pro in a trains-conformation), an NOE between ^{MN}P320 H^{α} and ^{MN}R322 H^{N}, and a strong sequential interaction between ^{MN}G321 H^{N} and ^{MN}P320 H^{α}. The φ and ψ angles of ^{MN}P320 are −72° and 65°, in excellent agreement with the characteristic inverse γ-turn angles (Creighton, supra). These differ markedly from the φ and ψ angles for a type II β-turn (−60° and 120°) and a type I β-turn (−60° and −30°). The side chain of ^{MN}R322 interacts extensively with the ^{MN}P320 and ^{MN}G319 residues that form the inverse γ-turn, thus defining the orientation of the Arg side chain with respect to the turn (rmsd of 0.74 Å for the best backbone superposition of ^{MN}K312-^{MN}T327).

### Example V

### The V3_{MN} Residues Interacting with the Antibody

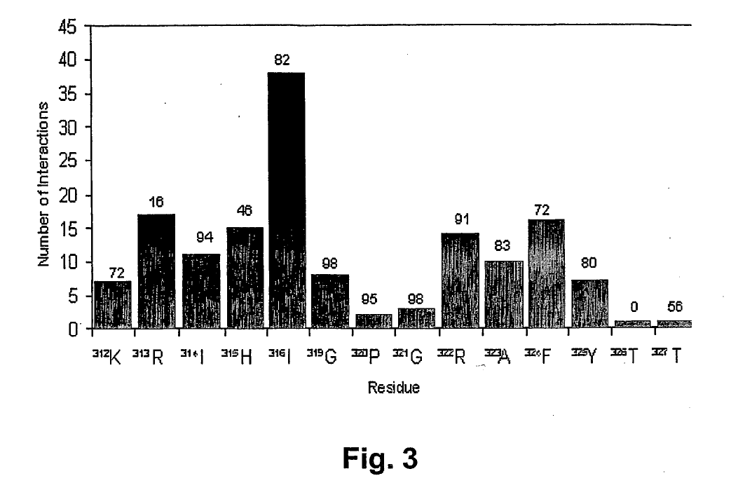

Extensive interactions between the peptide and 447Fv were observed in the ¹³C edited NOESY spectrum. As shown in FIG. 3, the N-terminal segment ^{MN}K312-^{MN}I316 contributes 59% of the peptide interactions with the Fv, with ^{MN}I316 contributing the largest number of interactions. The C-terminal segment comprising residues ^{MN}R322-^{MN}T327 contributes 33% of the interactions of the peptide with the Fv, significantly less than the N-terminal segment. The dominance of the N-terminal segment and ^{MN}R322 in the interactions with the 447Fv (68% of the interactions with the 447Fv) is supported by the dynamic-filtering experiments which showed that the segment ^{MN}K312-^{MN}R322 was strongly immobilized upon binding to the 447Fv. ^{MN}T326 and ^{MN}T327 have one interaction each with the Fv. The T₂ relaxation times of ^{MN}T326 and ^{MN}T327 are comparable to those of peptide residues that interact more extensively with the Fv, most probably because of interactions between the two β-strands in the β-hairpin involving these two residues (see above).

About half (55 out of 120) of the observed peptide side chain interactions with the Fv are with aromatic rings, indicating that the antibody binding site is rich in aromatic residues, as can be deduced from the sequence of the variable loops (Thompson, J et al. (1996) J. Mol. Biol. 256:77-88). The existence of this aromatic environment is also reflected in the unusual high-field chemical shift observed for the protons of ^{MN}G319, ^{MN}P320 and ^{MN}R322 caused by the local ring current fields induced by aromatic amino acid residues (Wüthrich, supra). As the antibody resonances have not yet been assigned, the peptide-Fv interactions could not be assigned to the specific 447Fv residues involved.

The amide protons of ^{MN}I314 and ^{MN}I316 were found to exchange slowly with the solvent and were detected even 24 h after exchanging H₂O with D₂O. All other amide protons disappeared due to fast exchange. This slow exchange of ^{MN}I314 and ^{MN}I316 amide protons indicated that they were protected from exchange with the solvent due to hydrogen bonding in the complex. As these two residues are not involved in hydrogen bonding within the β-hairpin, they must be involved in intermolecular bonds to the Fv. The slow exchange of the amide indicates a very tight binding of the V3_{MN} peptide to the 447Fv.

### Example VI

### The V3 Structure Bound to the 447Fv is Highly Homologous to β-Hairpins in CD8, MIP-1α, and RANTES

To reveal potential structural homologues for the V3 β-hairpin, the present inventors searched the Protein Data Bank (PDB) using the SPASM program (Kleywegt, G J (1999) J. Mol. Biol. 285:1887-1897) and found that out of 9848 β-hairpins that differed from the V3_{MN} β-hairpin by a backbone rmsd of less than 2.5 Å, 512 contained the peptide motif IxI (where x is any amino acid) or homologues thereof with conservative replacement of Ile by Leu or Val. Of the 6 V3 residues found herein to interact most extensively with the 447Fv, ^{MN}I314, ^{MN}I316 and ^{MN}R322 are the most conserved, with 94%, 82% and 91%, conservation, respectively (LaRosa, G J et al. (1990) Science 249:932-935).

Of the 512 β-hairpins, 54 and 60 had Arg or Lys separated by two or three residues, respectively, from the IxI motif or its conservative homologues.

In V3_{MN}, the two Ile residues are separated by His which interacts extensively with the antibody but is conserved in only 46% of HIV-1 isolates (LaRosa et al., supra). In a search in which the “x” residue in the IxI motif was restricted to an aromatic residue, but not tryptophan, ten β-hairpins were found to have the motif
(I/L/V) (H/F/Y) (I/L/V) x x x (R/K) [SEQ ID NO:35]

Three of these β-hairpin-containing proteins were of human origin, two of which were CD8 (Gao, G F et al. (1997) Nature 387:630-634; Leahy, D J et al (1992) Cell 68:1145-1162) (in free and complexed form), while the third was an Alzheimer's disease precursor protein (Zhang, Z et al. (1997) EMBO. J. 16:6141-6150). As shown in FIG. 4A the orientation of the two Ile residues and the positively charged side chain (K56 in CD8 and ^{MN}R322 in V3) is very similar in V3_{MN} and CD8. The alignment of CD8 and V3_{MN} sequences is presented in the description of FIG. 4A-4C. above.

The search revealed that only 7 of the homologous β-hairpins had the sequence (I/L/V) (H/F/Y) (I/L/V) x x (R/K) [SEQ ID NO:36]. Four that were of human origin included MSP-1α (Czaplewski, L G. et al. (1999) J. Biol. Chem. 274:16077-84), RANTES (Chung, C W et al. (1995) Biochemistry 34 9307-14), Met-RANTES (Hoover, D M et al. 1EQT, Cytokine, Met-RANTES, in PDB), and an oncogene product involved in T cell prolymphocytic leukemia.

The sequence alignment of MIP-1α and RANTES chemokines with the V3_{MN} peptide is shown in the description of FIG. 4A-4C, above. Of the 3 non-human β-hairpins, one was a synthetic anti-HIV protein (Wilken, J et al. (1999) Chem. Biol. 6:43-51) and the other was a viral macrophage inflammatory protein-II (v-MIP) (Fernandez, E J et al. (2000) Biochemistry 39:12837-44).

Five out of the seven peptides bind to R5. Although according to the homology search, the basic amino acid in RANTES and MIP-1α is separated by two residues from the (I/L/V) (H/F/Y) (I/L/V) motif (and separated by three residues in V3_{MN}), the side chain of the basic residue points in the same direction in the three proteins. The overall topology of the (I/L/V) (H/F/Y) (I/L/V) tripeptide is similar, as shown in FIG. 4B. (H is neutral and aromatic at pH above the imidazole's pKa.)

To test the uniqueness of the similarity between V3_{MN} and the corresponding β-hairpins in MIP-1α and RANTES, we repeated the search looking for homologous β-hairpins with a sequence (I/L/V) (H/F/Y) (I/L/V) x x x x (R/K) [SEQ ID NO:60]. Only human MIP-1α and RANTES and two rat proteins were identified, showing that the results are almost the same irrespective of whether the positively charged (R/K) residue is separated from the triad (I/L/V) (H/F/Y) (I/L/V) motif by two or four residues.

As shown in the Table under the description of FIG. 4A-4C, in MIP-1β, the residue following the aromatic residue is Gln instead of a non-polar aliphatic amino acid. However, the remaining three residues of the motif mentioned above are conserved

(I/L/V) (H/F/Y) x x x (R/K) and (I/L/V) (H/F/Y) x x x x x (R/K) [SEQ ID NO:38]

The sidechains of MIP-1β V40, F41, Q42 and R45 superimpose on the side chains of the corresponding residues ^{MN}I314, ^{MN}H315, ^{MN}I316 and ^{MN}R322 in V3_{MN}. The backbone rmsd between MIP-1β and the structure of V3_{MN} bound to 447Fv is 1.88 Å for the segment IHIGPGRAFY [SEQ ID NO:39], revealing the same structural homology between V3_{MN} and MIP-1β as that observed between V3_{MIN} and MIP-1α and RANTES.

### Example VII

### The V3 Structure Recognized by 0.5β Fv is Homologous to a β-Hairpin in SDF-1

The HIV IIIB strain has an atypical two residue insertion at positions ^{IIIB}Q317 and ^{IIIB}R318 of the V3 loop. This insertion does not affect the length of the β-strands in V3_{IIIB} bound to 0.5β Fv in comparison to V3_{MN} bound to 447Fv but rather creates a six residue loop comprising residues ^{IIIB}Q317, ^{IIIB}R318, ^{IIIB}G319, ^{IIIB}P320, ^{IIIB}G321 and ^{IIIB}R322 instead of the four residue GPGR loop in V3_{MN} (Tugarinov et al., supra). The reverse turn in V3_{IIIB} is shifted one residue upstream and comprises RGPG to maintain the central location of the reverse turn at the tip of the β-hairpin.

Therefore, to accommodate the two residues insertion, the coordinates for ^{IIIB}G319 and ^{IIIB}P320 at the tip of the turn were excluded when using the SPASM program to search the Protein Data Banks (PDB) for structural homologues of the V3_{IIIB} β-hairpin.

Of 5734 β-hairpins that differed from the V3_{IIIB} β-hairpin by a backbone rmsd less than 1.0 Å, only the structure of SDF-1 (Dealwis, C et al. (1998) Proc. Natl. Acad. Sci. USA 956941-46; Ohnishi, Y et al. (2000) J. Interferon Cytokine Res. 20:691-700) in the PDB contained the (I/L/V/A) (H/R/K) (I/L/V/A) motif. (H is included since it is positively charged at pH below the imidazole's pKa.) The backbone superposition of the SDF-1 β-hairpin over the V3_{IIIB} β-hairpin (FIG. 4C) shows an excellent fit with a backbone rmsd of 0.47 Å for the overlapping 13-residue segments:

| | |
|---|---|
| (a) KSIRIQR---RAFVTI of V3; | [SEQ ID NO:24] |
| and | |
| (b) IVARLKN---NRQVCI of SDF-1.. | [SEQ ID NO:25] |

(See table in description of FIG. 4A-4C.) Interestingly, residues ^{IIIB}G319 and ^{IIIB}P320 at the tip of the turn protruded beyond the common structure as a result of the longer loop. SDF-1 has an Arg residue after the reverse turn as does V3_{IIIB}, but this Arg is shifted one residue down stream in the SDF-1 sequence.

### Discussion of Examples I-VII

### The Neutralizing Face of the V3 Loop

The structure of a 23-residue HIV-1_{MN} V3 peptide bound to the Fv-fragment of the human mAb 447-52D in solution was solved using multidimensional heteronuclear NMR, and the interactions between the peptide and 447Fv were assigned to specific peptide residues. The V3 epitope ³¹²⁻³²⁷gp120 bound to 447Fv forms a β-hairpin consisting of two anti-parallel β-strands comprising residues ^{MN}R313-^{MN}I316 and ^{MN}A323-^{MN}T326 linked by an inverse GPG γ-turn (FIG. 2C). Residues ^{MN}K312, ^{MN}R313, ^{MN}I314, ^{MN}H315, ^{MN}I316 from the N-terminal strand and residues ^{MN}R322, ^{MN}A323, ^{MN}F324 and ^{MN}Y325 from the C-terminal strand form most of the intermolecular interactions of the V3_{MN} peptide with the 447Fv (FIG. 3). These residues form an exposed surface of the V3 loop that has the potential for interacting with the chemokine receptors CCR5 and CXCR4. Indeed, alanine scanning showed that V3 residues ^{MN}K312, ^{MN}I314, ^{MN}R322 and ^{MN}F324 are important for CCR5 binding (Wang et al., supra). Moreover, each of these residues is highly conserved: ^{MN}I314 is found in 94% of HIV-1 isolates, ^{MN}K312 and ^{MN}R322 are identical or conservatively replaced by Arg or Lys respectively, in 95% and 91.5% of HIV-1 isolates, and ^{MN}F324 is conserved in 71% of HIV-1 isolates (LaRosa et al., supra).

An earlier study by some of the present inventors showed that, in the complex of a V3_{IIIB} peptide bound to the strain-specific HIV-1 neutralizing murine mAb 0.5β, residues ^{IIIB}I314, ^{IIIB}R315, ^{IIIB}Q317, ^{IIIB}P320, ^{IIIB}R322 and ^{IIIB}F324 formed most of the interactions with the antibody (Tugarinov, V et al. (2000) Structure Fold. Des. 8:385-395). Thus, the NMR studies to date indicate that the N-terminal segment close to the tip of the V3-loop, and to a lesser extent the C-terminal segment following the GPG sequence, are recognized by HIV-1 neutralizing anti-V3 antibodies.

The crystal structure of V3_{MN} peptides complexed with one of three different murine mAbs (50.1, 59.1 and 58.2), elicited against a cyclic peptide comprising the entire V3 loop was solved by X-ray crystallography performed by Ian Wilson's group and by others (Ghiara et al., 1994; Rini et al., supra; Stanfield et al., supra, WO 94/18232, 1994). MAb 50.1 interacts with the segment ^{MN}K312-^{MN}P320, MAb 59.1 interacts with ^{MN}I316-^{MN}F324 and MAb 58.2 interacts with ^{MN}R313-^{MN}Y325. The “combined” epitope recognized by the three anti-peptide murine mAbs overlaps the epitope recognized by the human mAb 447-52D, excluding ^{MN}T326 and ^{MN}T327. While 59.1 and 58.2 showed no obvious preference for interaction with the N-terminal strand of the epitope, MAb 50.1 interacted only with the N-terminal strand and the beginning of the turn.

### The β-Hairpin is a Common Structural Feature of V3 Loops of Different HIV-1 Strains

Since mAb 447-52D was elicited against the HIV-1 virus, binds to intact virions (Nyambi et al., supra), and neutralizes a broad spectrum of viruses (Gorny et al., 2002 supra), it is concluded that it recognizes V3 in a conformation that exists naturally on the virus particles. When a flexible V3 peptide binds to such an antibody, it assumes the conformation that resembles the conformation against which this antibody was originally elicited.

MAb 0.5β studied previously by several of the present inventors was elicited against a soluble gp120 protein and therefore recognizes a V3 loop conformation that exists in the context of this whole protein (Tugarinov et al., 1999, supra). In contrast, the linear V3_{MN} peptides as well as its cyclized form that served as the immunogen to induce the three anti-peptide antibodies used by Wilson's group in obtaining the crystal structures (supra) are mostly flexible and, except for a S-turn in the GPGR segment, do not, show any detectable secondary structure in aqueous solution (Chandrasekhar et al., supra).

The present inventors' group previously observed a β-hairpin conformation with a type VI β-turn in the V3_{IIIB} peptide bound to 0.5β Fv (Tugarinov et al., supra). Therefore, both these earlier findings, and those disclosed herein, suggest that the β-hairpin structure is conserved in the V3 region of gp120 from different virus strains. This conclusion is consistent with the prediction that the V3 loop of most HIV-1 strains forms a β-strand, β-turn, and β-strand conformation (Hansen, J E et al. (1996) Proteins 25:1-11; LaRosa et al., 1990).

It is important to note that while the reported X-ray structures agree with the secondary structure predictions with respect to the N-terminal segment of V3, they either provide no information or indicate different structures (multiple turns) for the C-terminal residues of V3 (Ghiara et al., 1994; Stanfield et al., Supra).

### The Flexibility of the GPGR Sequence [SEQ ID NO:3]

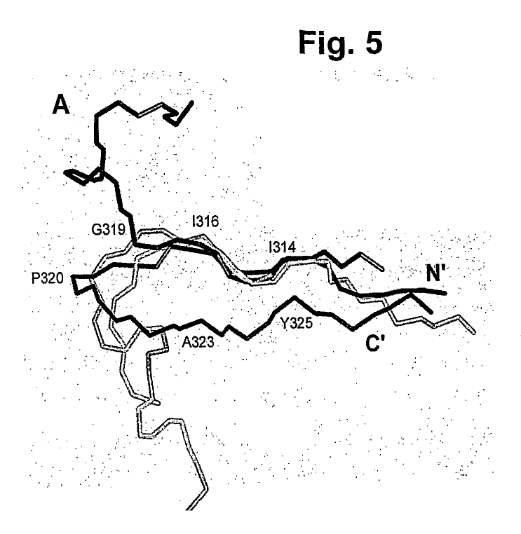

As summarized in FIG. 5A (and the table in the description of FIG. 5), the GPGR segment adopts different types of reverse turns when bound to different HIV-1 antibodies. The inverse γ-turn in V3_{MN} bound to 447Fv differs from (a) the type II and type-I β-turns in the V3_{MN} peptide bound to the three murine anti-peptide mAbs antibodies, and (b) the type VI cis-proline β-turn that was observed in the GPGR segment at the center of a V3_{IIIB} peptide bound to the 0.5β Fv (Tugarinov et al., supra). The existence of these different types of turns suggests a conformational flexibility in the GPGR sequence, a flexibility that is likely conferred by the two glycine residues flanking the proline. As a result of this flexibility, the GPGR segment may adopt different conformations according to the remaining sequence of the V3 loop. Thus, the (rare) QR insertion that precedes the GPGR in V3_{IIIB} caused a shift in the β-turn in order to preserve the alignment of the hydrogen bonds in the O-hairpin (Tugarinov et al., supra). This may conserve the spatial arrangement of the side chains critical for binding to the chemokine receptors.

In addition, conformational flexibility of the V3 loop may contribute to the topology of the β-hairpin surface exposed to the HIV coreceptors and allow the V3 region to optimize its conformation to maximize its binding to one or more of the chemokine receptors (pee below).

### Alternative Conformations of the V3 Loop

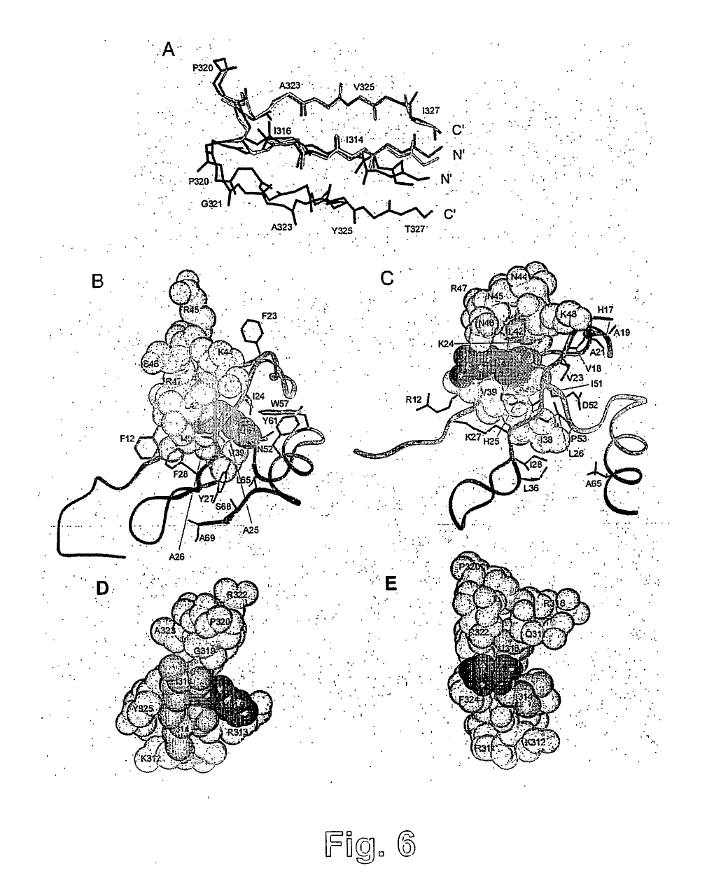

Although both V3_{IIIB} bound to 0.5β Fv and V3_{MN} bound to 447Fv form β-hairpins, these two differ in the network of hydrogen bonds that stabilize the β-hairpin conformation. Whereas in the V3_{IIIB} peptide, ^{IIIB}K312, ^{IIIB}I314 and ^{IIIB}I316 form hydrogen bonds with ^{IIIB}I327, ^{IIIB}V325 and ^{IIIB}A323, respectively, in the V3_{MN} peptide there is a one residue shift in the intra-peptide hydrogen bonds, such that ^{MN}R313 and ^{MN}H315 form hydrogen bonds with ^{MN}T326 and ^{MN}F324, respectively. As a result of this shift, side chains pointing upward in V3_{IIIB} point downwards in V3_{MN}, as if the two conformations were related by an imaginary inversion axis. As shown in FIG. 6A, superpositioning of the IHI segment of V3_{MN} over the corresponding IRI segment in V3_{IIIB} results in a 180° difference in the direction of the continuation of the β-turn and the second β-strand. In the context of the larger gp120 protein, residues exposed in one type of β-hairpin may be buried in the other and vice versa. These differences in orientation may have profound implications on co-receptor binding and antibody binding.

Superpositioning of the IFL segment in MIP-1α, the VFV segment in RANTES and the VFQ segment in MIP-1β over the ARL motif of SDF-1 created the same 180° rotation as that observed between V3_{MN} and V3_{IIIB} (FIG. 6A). The effect of this change in conformation is illustrated in a comparison of MIP-1α and SDF-1 (FIGS. 6B and 6C). The ribbon diagrams (in red) show that the overall spatial orientation of the chemokines' backbones is quite similar. Nevertheless, the space-filling blow up of the β-hairpins clearly shows that in MIP-1α, residue F41 is buried while I40 and L42 are exposed (FIG. 6B). In SDF-1, a 180° flip results in the exposure of residue R41 and the burial of residues A40 and L42 (FIG. 6C). It is evident that the topology of the side chains of these amino acid triads is inverted in these two chemokines.

The topological relationship of the V3 loop with respect to native gp120 is unknown. FIGS. 6D and 6E show the orientation of V3_{MN} and V3_{IIIB} obtained by superpositioning them over the homologous β-hairpins in MIP-1α and SDF-1, respectively. This superpositioning shows a remarkable resemblance in the orientation of the triad residues between V3_{MN} (IHI) and MIP-1α (IFL), and between V3_{IIIB} (IRI) and SDF-1 (ARL). As with MIP-1α and SDF-1, the orientation of the sidechains of the V3_{MN} triad IHI is inverted in comparison to the orientation of the side chains of the V3_{IIIB} IRI triad (FIGS. 6D and 6E). Thus, the alternative conformations found in the V3_{MN} versus V3_{IIIB} β-hairpins are highly analogous to the alternative β-hairpin conformations found in MIP-1α/MIP-1β/RANTES versus SDF-1, respectively.

As noted above, varying conformations of the V3 loop around the GPGR β-turn were observed by Wilson's group comparing three different anti-V3 peptide antibodies complexed with a V3_{MN} peptide. However, the β-hairpin conformation was not observed (Stanfield et al., supra). This did not allow these workers to observe the one residue shift in the hydrogen bond network discovered by the present inventors and its implications for appreciating different side chain orientations and surface topologies that are possible.

### Homology Between the V3 Loop and β-Hairpin Structures of R5 and X4 Chemokines

The correlation between the conformation of V3_{MN} bound to the 447Fv and the β-hairpins in MIP-1α, MIP-1β and RANTES suggests that this particular conformation of V3 that is recognized by 447Fv is the conformation that interacts “naturally” with CCR5. Thus, the VFV motif in RANTES is part of the β2-strand (residues 38-43) which forms a β-sheet with the β1-strand of the protein. Both β-strands are implicated in binding to R5 (Nardese, V et al. (2001) Nat. Struct. Biol. 8:611-615). The corresponding region of the V3 loop also participates in chemokine binding (Wang et al., supra).

The observation that affinity-purified anti-V3 antibodies isolated from HIV-1-infected patients cross-react with MIP-1α and RANTES (Kissler, S et al. (1997) Clin. Immunol. Immunopath. 84:338-341) further supports the apparent homology between the structures of V3 and these chemokines. However, since the sequence identity between the 447-52D epitope and the corresponding region in MIP-1α is only 7%, it is unlikely that a mAb such as 447-52D will cross-react with MIP-1α, MIP-1β and RANTES.

The relationship between the structure of V3_{IIIB} bound to 0.5β mAb and that of SDF-1 (FIGS. 6A-6E) also suggests that the conformation of bound V3_{IIIB} is representative of the V3 loop in the X4 subgroup of HIV-1 viruses.

Further evidence supporting the proposed homology between V3 and the β-hairpin structures of the chemokines which are believed to be the physiological ligands for HIV-1 co-receptors comes from several investigators who have shown that V3 loop-derived peptides can inhibit viral entry into target cells in a co-receptor specific manner (Basmaciogullari S et al., 2002, J Virol. 76:10791-800; Sakaida, H et al., 1998, J Virol. 72:9763-70; Verrier et al., supra).

### The Mechanism for Co-Receptor Selectivity

Four residues implicated in CCR5 binding (K312, I314, R322 and F324) are included in the V3 β-hairpin (Wang et al., supra) that is bound by 447-52D. The orientation of each of these amino acids is reversed in the β-hairpin conformations of bound V3_{MN} when compared to antibody-bound V3_{IIIB}. It is therefore difficult to envision how these alternative conformations could bind to the same receptor. If V3_{MN} bound to 447Fv is in an R5 virus conformation, while the V3_{IIIB} bound to 0.5p is in an X4 virus conformation, the differences in these critical residues could account for co-receptor selectivity.

As noted above, the overall spatial arrangements of the backbones of MIP-1α/MIP-1β/RANTES and SDF-1 show significant homology (FIGS. 6B and 6C), and common sequence motifs appear in both. Further analogies emerge upon inspection of their electrostatic properties. As seen in FIG. 6C, the β1-strand adjacent to the ARL motif of SDF-1 contains positively charged residues K24, K27, and H25 (the latter depending on pKa). In contrast, in MP-1α, I24, A25 and Y27 occupy the corresponding positions of the β1-strand (FIG. 6B), creating a neutral and more hydrophobic surface adjacent to the I40-F41-L42 triad in the 3D structure of the protein. An increased positive charge at the base of the V3 loop is known to “convert” an R5 virus into an X4 virus. The residue that is very important for co-receptor selectivity is #329. This residue is two residues C-terminal of the β-hairpin C-terminus; mutation of this residue from D to R results in an isolate that has switched from being R5 to being X4 (De Jong et al., supra).

Placing a positively charged residue at this position in V3 may change the charge of the surface so that it mimics the positively charged β1 strand in SDF-1 (see above). If this is correct, it suggests that increased positivity and β-hairpin conformation mimicking the SDF-1 surface is involved in CXCR4 binding, while a less positive surface and a MIP-1α-like β-hairpin conformation mimics the MIP-1α and RANTES surface that binds to CCR5.

Again, the 447-52D antibody arose in an HIV-1 infected individual and, therefore, we will never know the exact viral strain and V3 sequence responsible for its production. Antibody 447-52D neutralizes a broad spectrum of HIV-1 isolates from different clades including primary X4 and R5 viruses. The epitope recognized by 447-52D does not include residue 329 which is the most crucial for co-receptor selectivity. Moreover, the consensus sequence of clade B R5 viruses in the region of the 447-52D epitope (³¹²⁻³²⁷gp120) differs by only one residue from the HIV_{MN} sequence: R313 in MN is replaced by Ser in R5 viruses). This replacement does not seem to interfere with 447-52D binding, since V3_{IIIB} also contains this replacement and HIV-1_{IIIB} is neutralized by this mAb (Gorny et al., 1993, supra). The importance of residue 313 in coreceptor selectivity but its minor effect, if any, on 447-52D binding could result from this residue being at the periphery of the 447-52D epitope.

Since V3 peptides are flexible and since the V3 loop of X4 and R5 viruses may differ only slightly in the epitope recognized by HIV-1 neutralizing antibody, the present inventors conceived that binding of the antibody induces the peptide to adopt that conformation that originally induced the antibody. That being the case, it should not matter whether the peptide used to form the antibody complex is from an X4 or an R5 virus. This explains why the V3_{MN} peptide, which represents the V3 sequence of an X4 virus, binds to the 447Fv in an “R5 topology.”

Ultimate proof of the involvement of such conformational changes in co-receptor selectivity will come with determination of the structure of gp120 (including V3) complexed with X4 and R5 chemokine receptor. Such experiments are currently not feasible due to difficulties in crystallizing membrane proteins. Nevertheless, the present structural studies of the V3 loop bound to neutralizing antibodies and studies of the natural ligands of the HIV-1 coreceptors provide compelling data that illuminate the mechanisms underlying coreceptor selectivity.

It is not altogether clear how 447-52D can neutralize both X4 and R5 viruses. One explanation is the existence of an equilibrium between the two V3 β-hairpin conformations with both present under physiological conditions. The selection of co-receptor is dictated mostly by residue 329 and to a lesser extent by the charges of residues 312 and 313. Since residue 329 is outside the epitope recognized by 447-52D, the antibody can neutralize both X4 and R5 viruses share sequences in the 447-52D epitope (³¹²⁻³²⁷gp120). Because of its conformational flexibility, once V3 binds 447-52D, the equilibrium would be shifted to the R5. It is noteworthy that in the C chemokine lymphotactin, an equilibrium between two β-hairpin conformations differing in their pattern of hydrogen bonds was observed in NMR studies (Kuloglu, E S et al. (2002) J. Biol. Chem. 277:17863-70). This serves as strong evidence for the feasibility of the V3 flexibility model of the present invention. This dual conformation of the lymphotactin β-hairpin was accompanied by a shift of one residue in the pattern of hydrogen bonds with a third β-strand that forms a 3-strand β-sheet with the hairpin. Similar interactions between V3 and other regions of gp120 could play a role in the conformational equilibrium of V3.

### Conclusions

The foregoing structural analysis using human antibodies raised against gp120 or against HIV-1 and specific for V3 epitopes show that the V3 loop can assume two types of β-hairpin structures that differ in the network of hydrogen bonds by a one residue shift. This results in a highly distinct orientation and exposure of the V3 residues among the two V3 conformations even though the sequence of the 10 central residues of V3 is highly conserved. One type of β-hairpin shows conformational and sequence similarity to the β-hairpin structures of MIP-1α, MIP-1β and RANTES that are implicated in R5 binding. The other V3 β-hairpin conformation resembles a β-hairpin in SDF-1 which binds to R4. According to this invention, the dual V3 conformations play a role in co-receptor selectivity.

### Example VIII

### Confirmation of Peptide Structure from NMR Study by X-Ray

In the following section, reference to amino acid residues with superscripted H and L numbers refer to residues in the antibody heavy (H) and light (L) chains. Numbers with a superscripted P refer to residues in the V3 peptide bound to the antibody.

This study was a collaboration between one of the present inventors and other collaborators (Stanfield, R et al. (2004) Structure 12:1-20) and is incorporated by reference.

The 16-mer peptide used for co-crystallization was CKRIHI--GPGRAFYTTC-NH₂; [SEQ ID NO:40] (previously termed MP1) which has residues 305-309 and 312-320 of the MN V3 sequence with a Cys added at each terminus. Residue positions 310 and 311 represent a gap. Unless the rest of this document, residue numbering in this Example is based on the sequence of the HXB2 strain of HIV-1 (Ratner, L et al. (1987). AIDS Res. Hum. Retroviruses 3:57-69). Residues P305-P316 (KRIHI--GPGRA [SEQ ID NO:41]) could be clearly interpreted in the electron density maps (except for the Lys^{P305} side chain). Weak electron density corresponding to three additional residues at the C-terminus (FYT, P317-319) was found, but despite repeated attempts, these residues could not be positioned with confidence.

Peptide residues KRIHI [SEQ ID NO:9] form an extended β-strand, followed by a type-II β turn around GPGR. The peptide β-strand surprisingly formed extensive main-chain interactions with the antibody-derived CDR H3 resulting in a 3-stranded mixed β-sheet, with an up/down/down topology and a standard left-handed twist. The β-sheet had one largely polar face consisting of Phe^{H97}, Met^{H99}, Arg^{H100a}, Asp^{H100f}, Tyr^{H100h}, Tyr^{H100j}, Arg^{P306}, His^{P308}, and Arg^{P315}, and on the other side, a more hydrophobic face coated by the side chains of Ile^{H98}, Ile^{H100}, Tyr^{H100g}, Tyr^{H100i}, Ile^{P307}, and Ile^{P309}.

Six hydrogen bonds and one salt-bridge were made between peptide and Fab 447, all to CDR H3. The salt-bridge between Asp^{H95} Oδ2 and Arg^{P315} NH1 (3.3; 3.4 Å; molecule 1 and 2) anchored the peptide to the base of H3. The only side-chain hydrogen bond was between Tyr^{H100j}OH and His^{P305}Nδ1 (3.0; 3.1 Å). The remaining five hydrogen bonds were between the peptide main-chain atoms and the Fab CDR H3 main chain in the β-sheet interaction (Asp^{H100f}N-Lys^{P305}O, 3.4; 3.4 Å; Asp^{H100f}O-Ile^{P307}N, 2.6; 2.9 Å; Tyr^{H100h}N-Ile^{P307}O; 2.7; 2.8 Å; Tyr^{H100h}O-Ile^{P309}N, 2.6; 2.9 Å; Tyr^{H100j}N-Ile^{P309}O, 3.0; 3.1 Å). Arg^{P315} made cation-π interactions with Trp^{H33} and Tyr^{H100j}, where Arg Nε was 3.8 Å from the center of the Tyr^{H100j} ring and 3.6-3.8 Å from the center of the aromatic ring of the Trp^{H33} indole. The Arg^{P315} guanadinium moiety was nearly co-planar with the Trp^{H33} indole (interplanar angle of 11; 16°) and Tyr^{H100j} ring (7; 13°). Hydrophobic interactions were made by Ile^{P307} and Ile^{P309} with Fab residues Tyr^{H100i} and Tyr^{H100g}. The Pro^{P313} side chain in the GPGR turn was about 3.6 Å from the Trp^{L91} indole, with the rings nearly co-planar (interplanar angle of 7; 7°), and about 3.7 Å from Trp^{L96} with the respective rings nearly perpendicular (interplanar angle of 76,800).

In the Fab-peptide complex, molecular surface areas of 555; 585 Å² (molecule 1, molecule 2) were buried on the Fab and 478; 494 Å² on the peptide. The majority (77%) of the buried Fab surface was contributed by the heavy chain (59% from H3), and is composed mainly of Tyr, Trp, Asp, and Glu side chains (43, 30, 9 and 8% of the surface, respectively). The peptide fit snugly into its binding site, with only one unfilled cavity near Ile^{P309}). A total of 116; 162 van der Waals contacts were made between peptide and Fab, with the majority from H3 (55; 43%), L3 (20; 33%), and H1 (21; 20%), and few to no interactions with L1 (2; 4%), H2 (1; 0%), and L2 (0; 0%). The differences in the van der Waals contacts in the two complexes is due to a slight variation in the position of the peptide (˜0.5 Å) relative to each Fab. Otherwise, the structures are very similar as reflected by corresponding RMSD's in Cα position for V_{L}, V_{H} (H1-H113) and peptide (P305-P316) of 0.12 Å, 0.22 Å, and 0.77 Å, respectively, when the V_{L} domains (L1-L107) are superimposed. The corresponding superposition on V_{H} domains results in RMSD's in Cα for V_{L}, V_{H}, and peptide of 0.23 Å, 0.12 Å, and 0.64 Å, respectively.

Peptide Binding Motif

Previous epitope analysis using overlapping peptides indicated that the core epitope of 447-52D was GPxR (Gorny et al., 1992, supra) which agrees well with the crystal structure, where the highly conserved β-turn crown (GPGR) is inserted into the heart of the 447-52D combining site, with its extended region (IRIHI; SEQ ID NO:42) interacting with antibody through extensive main-chain hydrogen bonding to the CDR H3 backbone, resulting in a composite 3-stranded β-sheet. Thus, main-chain interactions dominate peptide binding to 447-52D, so that side-chain substitutions at many positions in the peptide can easily be accommodated. Specific interactions with Pro^{P313} and Arg^{P315} at the base of the binding site likely confer specificity for V3-like sequences despite the non-specific nature of the main-chain interactions. This conclusion is consistent with screening of 447-52D against a 15-mer phage-display library (Keller et al., 1993), which showed that of 55 binding peptides, Gly, Pro, and Arg were always selected at P312, P313, and P315, respectively. On the other hand, 447-52D could bind peptides with many different residues at P308, with the most frequent being Leu (15/55), His (9/55), Phe (6/55), Arg (5/55) and Tyr (5/55), indicating that the His^{P308} hydrogen bond seen in the crystal structure is not critical for peptide binding to 447-52D. However, position P309 is more restricted to hydrophobic residues, with Phe (17/55), Tyr (12/55), Ile (8/55), Val (7/55) and Leu (7/55) appearing most frequently. At P314, Gly (30/55) and Ala (10/55) are strongly preferred, but Ser, His, Lys, Leu, Asn, Gln, and Arg can also be tolerated in the phage display peptides. A slight preference was found for Gly (11/55) at position 316, although perhaps surprisingly, many other residues were tolerated. Gly^{P316} (the i+2 residue in the type II β-turn) has torsion angles of φ=74°, ψ=5°, in the left-handed α-helical region of the Ramachandran plot. Thus, substitution at P316 with a non-Gly residue might be expected to change the turn type by flipping the P316 carbonyl. The carbonyl makes no hydrogen bonds to the antibody in the present structure, and there is ample room to accommodate this flip should it take place. Otherwise, no strong preferences are found at positions prior to P308 or after P316. Thus, it appeared that only the V3 GPxR crown residues are highly restricted in sequence preference, with little or no specific requirements at other positions. The strong preference for Arg at position P315 is also in agreement with neutralization data, where the non-clade B primary isolates that are neutralized by 447-52D retain an Arg at this position. However, most non-B viruses have a Gln at position P315, and it is not yet known whether the Gln substitution in the V3 crown can be recognized by 447-52D.

### Example IX

### Solution Structure of the 447 Antibody-Bound V3_{IIIB} Peptide

### Mapping the V3_{IIIB} Epitope Bound to 447-52D

A sample of ¹⁵N-labeled V3_{IIIB} peptide, ³¹⁰⁻³²⁹gp120_{IIIB} (TRKSIRIQRGPGRAFVTIGK [SEQ ID NO:37]), in complex with unlabeled 447-52D Fv was prepared and T₂ ¹⁵N relaxation times were measured. Relaxation rates of nuclear spin magnetization are a function of the molecular mobility, and therefore can be used to extract information on the internal dynamics of the Fv-bound peptide. Short ¹⁵N T₂ relaxation times (<100 msec) were found for ^{IIB}K312-^{IIIB}G328, indicating that this segment comprises the V3_{IIIB} epitope recognized by 447-52D.

The definition of the epitope was further confirmed by Fv-induced changes in a ¹H-15 HSQC spectra of the peptide in its free and Fv-bound forms, and by examination of peak intensities in the bound state. Comparison of the two spectra of the free and the Fv-bound peptide revealed that the chemical shift of ^{IIIB}K329 did not change upon binding, implying that ^{IIIB}K329 does not interact with 447-52D Fv and is outside the epitope recognized by 447-52D. Narrow linewidth in the spectrum of the bound peptide, characteristic of mobile residues, were observed only for residue ^{IIIB}K329. The absence of observable cross peaks for residues ^{IIIB}T310 and ^{IIIB}R311 at the N-terminal of the peptide in all ¹H-¹⁵N correlation spectra results from rapid exchange of their amide protons with the solvent, proving that these residues do not interact with the 447-52D antibody.

### Solution Structure of the V3_{IIIB} Peptide Bound to 447-52D Fv

The structure of the V3_{IIIB} peptide bound to 447-52D Fv was determined using 365 NMR-derived distance (75 long- and medium-range), 21 dihedral angle, and 5 hydrogen bond constraints. The superposition of the 29 lowest-energy structures that satisfy the experimental restraints with no NOE violations larger than 0.4 Å is shown in FIG. 9A. A ribbon representation of the bound peptide structure is shown in FIG. 9B. The bound peptide forms a structurally well-defined β hairpin consisting of two antiparallel β-strands made of residues ^{IIIB}S313-^{IIIB}I316 and ^{IIIB}F324-^{IIIB}I327. However, lower structure definition is observed for the loop region. The root-mean-square deviation (rmsd) values for the entire epitope (³¹²⁻³²⁸gp120) are 0.58 Å and 1.31 Å, and for the β-strands 0.30 Å and 0.89 Å, for the backbone and heavy atoms, respectively. The structural statistics and rmsd values are presented in Table 2. The Ramachandran plot (not shown) of the mean structure of the V3_{IIIB} peptide bound to 447-52D Fv suggests that the φ and ψ angles of the peptide residues predominantly occupy allowed regions.

The average NMR coordinates for the V3_{IIIB} peptide as bound to and constrained by the 447Fv antibody fragment are shown in Table 4. The individual values for the 29 lowest energy structures will be deposited in the PDB and publicly available.

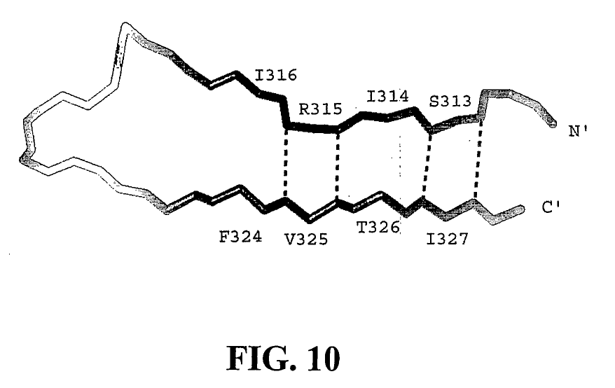

As shown in FIG. 10, the epitope forms a β hairpin consisting of two antiparallel β strands formed by residues ^{IIIB}S313-^{IIIB}I316 and ^{IIIB}F324-^{IIIB}I327.

NOE interactions characteristic of a β-hairpin conformation were observed between backbone atoms of the N-terminal and C-terminal halves. These interactions include ^{IIIB}R315 H^{N}/^{IIIB}V325 H^{N}, ^{IIIB}I316 H^{α}/^{IIIB}V325 H_{N}, ^{IIIB}Q317 H^{N}/^{IIIB}F324 H^{α}, ^{IIIB}K312H^{α}/^{IIIB}G328 H^{α}, ^{IIIB}I314 H^{α}/^{IIIB}T326 H^{α} and ^{IIIB}I316 H^{α}/^{IIIB}F324 H^{α}. The expected ^{IIIB}S313 H^{N}/^{IIIB}I327 H^{N} and ^{IIIB}R315 H^{N}/^{IIIB}T326 H^{α} NOE interactions could not be assigned because of resonance overlap. ³J_{HNHα} coupling constants higher than 8.4 Hz, typical of a β strand, were measured for ^{IIIB}S313, ^{IIIB}I314, ^{IIIB}R315, ^{IIIB}I316, ^{IIIB}V325, ^{IIIB}T326 and ^{IIIB}I327.

The β hairpin of the V3 epitope (³¹²⁻³²⁸gp120) is stabilized by a network of hydrogen bonds between the two strands (FIG. 10). Two pairs of hydrogen bonds are formed between ^{IIIB}S313 and ^{IIIB}I327 and between ^{IIIB}R315 and ^{IIIB}V325. The side chains of residues ^{IIIB}S313, ^{IIIB}R315, ^{IIIB}V325, and ^{IIIB}I327 form the lower face of the β hairpin, while the side chains of ^{IIIB}I314, ^{IIIB}I316, ^{IIIB}F324, and ^{IIIB}T326 form the upper face.

V3_{IIIB} Interactions with the Antibody

The N-terminal segment ^{IIIB}K312-^{IIIB}I316 was found to contribute approximately 60% of the peptide NOE interactions with the Fv, with ^{IIIB}I316 involved in the largest number of interactions. A similar pattern of intermolecular NOEs has been previously observed in V3_{MN} complex with 447-52D Fv. Moreover, in 25% of these interactions, almost identical Fv proton chemical shifts were observed in the 447Fv/V3_{MN} and 447Fv/V3_{IIIB} complexes, indicating a similar manner of Fv engagement with both peptides. Significantly, practically all these comparable NOEs originated from the N-terminal β strand. The Fv protons have not yet been assigned by NMR, however several of the similar chemical shifts are characteristic of aromatic residues, and particularly tyrosines. The presence of aromatic residues in the binding site is also reflected in the unusual high-field chemical shift observed for the protons of ^{IIIB}G319 and ^{IIIB}P320 caused by the local ring current fields induced by aromatic amino acid residues (Wuthrich, supra). Further support for the involvement of aromatic residues in V3 binding was obtained from the crystal structure of 447 Fab in complex with V3_{MN} solved recently by X-ray crystallography (by one of the present inventors and coworkers; see also Example VIII). Therefore, the V3_{IIIB} peptide is believed to interact with the same Tyr residues as does V3_{MN}.

### The Long Loop of Bound V3_{IIIB}

The loop linking the two V3_{IIIB} β-strands strands comprising of 7 residues (^{IIIB}Q317-^{IIIB}A323) is longer than that observed in the V3_{MN} peptide bound to 447-52D Fv. The conformation of the loop is stabilized by an i,i+3 hydrogen bond, between the carbonyl oxygen of ^{IIIB}G319 and the amide proton of ^{IIIB}R322. The structure of the loop is not as well defined as that of the β-strands, due to the small number of distance restraints. Within the 29 lowest-energy structures (FIG. 10A), several types of turns were found, indicating the divergence of the loop regions. The rare QR insertion N-terminal to the GPGR sequence that is found in HIV-1_{IIIB} strain could explain the observation of a longer loop which may not fit perfectly into the antibody's binding site, resulting in loose binding and increased mobility. The association constant of 447-V3_{IIIB} is one order of magnitude lower than the affinity of 447 for the corresponding V3_{MN} peptide. This suggests less optimal binding of the V3_{IIIB} peptide.

### Comparison Between the V3_{MN} and V3_{IIIB} Bound to 447 Fv

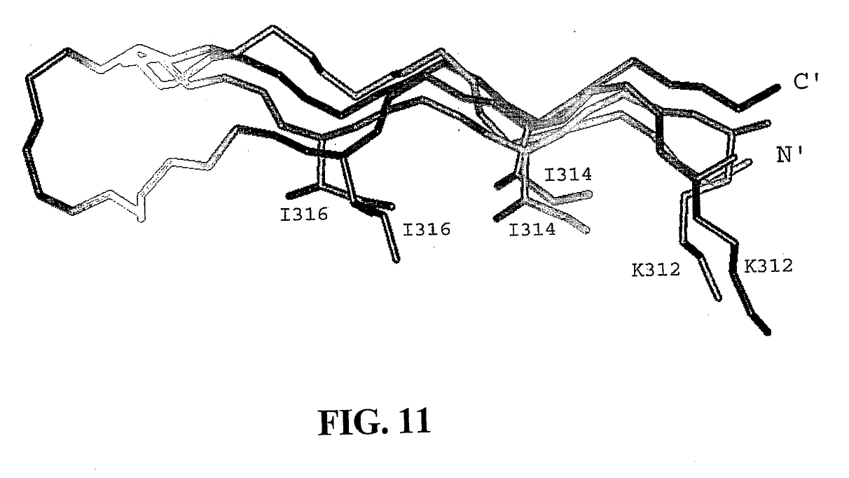

The structures of the V3_{IIIB} and V3_{MN} peptides bound to 447Fv were superimposed for best fit in their N-terminal β-strand (segment ³¹²KSIRI³¹⁶ and ³¹²KRIHI³¹⁶, respectively). As is shown in FIG. 11, the N-terminal β-strand in both peptides had the same conformation and the C-terminal β-strand continued in the same direction. In both 447Fv-bound peptides, the side chains of K312, I314 and I316 were similarly oriented and both I314 and I316 formed extensive interactions with 447Fv, the latter contributing most to the antibody-antigen interactions. In both 447Fv bound peptides residues 313 and 315 form intra-peptide hydrogen bonds. This observation emphasizes the importance of the N-terminal segment, and the key role of K312, I314 and I316, in binding to the broadly cross reactive antibody 447-52D.

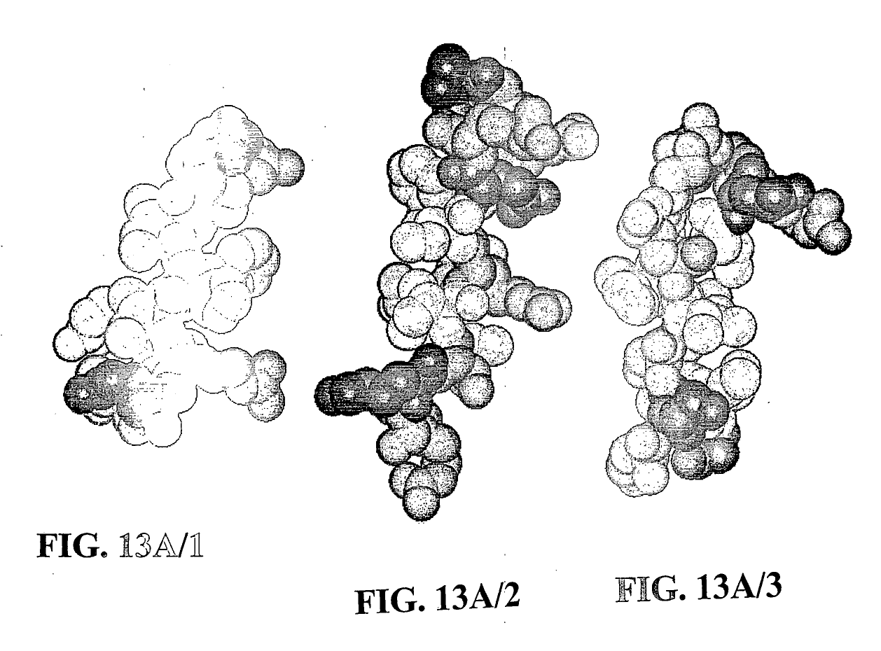

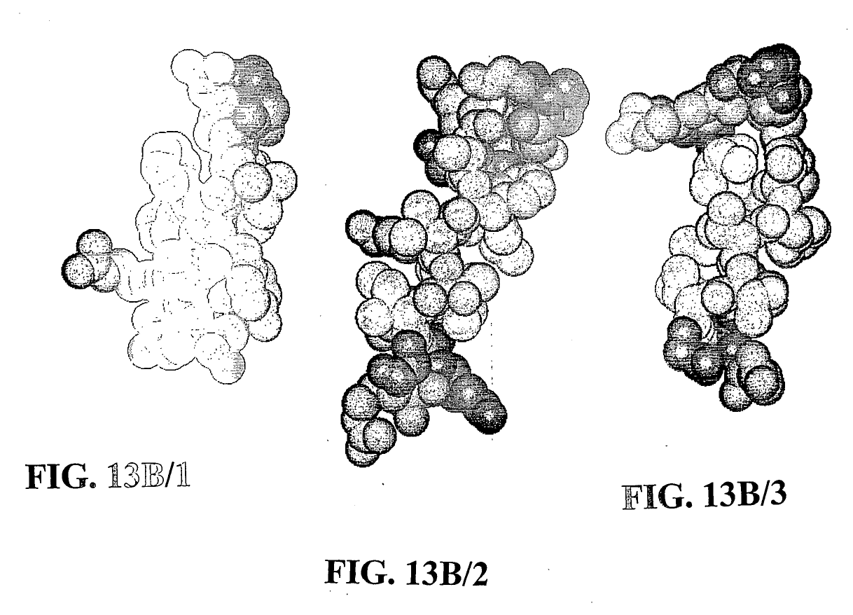

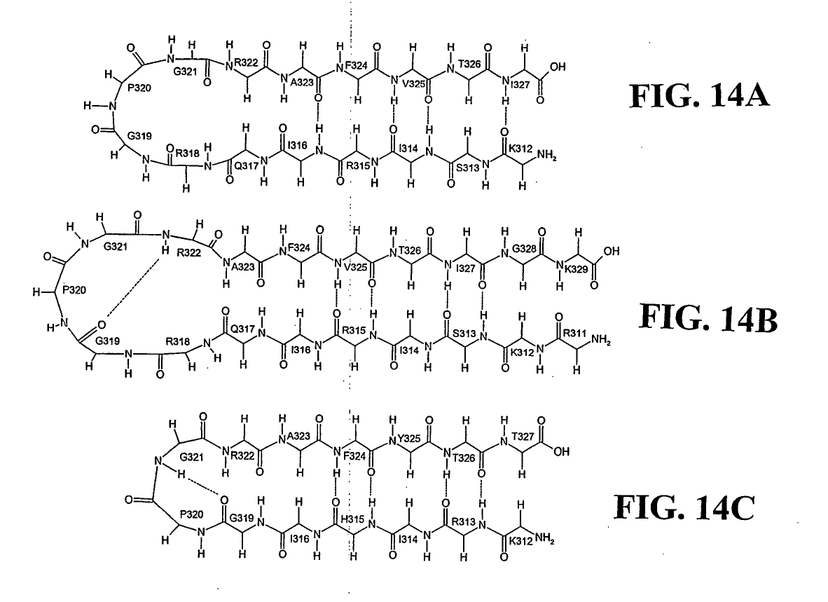

However, the C-terminal β-strands of V3_{IIIB} and V3_{MN} bound to 447-Fv were found to be remarkably different. When the two bound 447 peptides were superimposed for best fit of the β-strands (³¹²KSIRI³¹⁶ [SEQ ID NO:15] and ³²⁴FVTI³²⁷ [SEQ ID NO:43] of V3_{IIIB} with ³¹²KRIHI³¹⁶ [SEQ ID NO:9] and 324FVTI³²⁷ [SEQ ID NO:44] of V3_{MN}), the resultant RMSD was relatively high (2.18 Å). This is due to the difference in the conformation of C-terminal β-strand. Whereas in V3_{MN} residues F324 and T326 form hydrogen bonds with residues 315 and 313, respectively, the same hydrogen bonds in V3_{IIIB} involve residues. V325 and I327, a shift of one residue (FIGS. 14B-14C). Hence, the topologies of the side chains of the N-terminal β-strands in 447Fv-bound V3_{IIIB} and V3_{MN} are homologous, while the topologies of the side chains of the C-terminal β-strands are very distinct (FIG. 13A/13B).

### Comparison of V3_{IIIB}-447Fv and V3_{IIIB}-0.5βFv

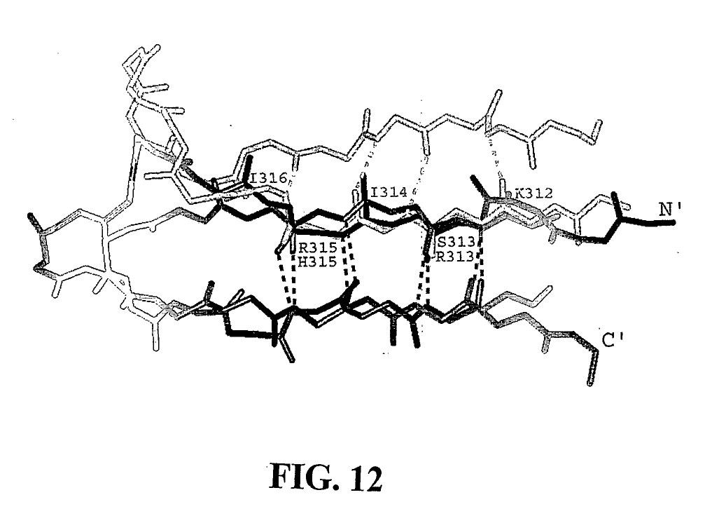

The N-terminal β-strands, ³¹³SIRI³¹⁶ of V3_{IIIB} bound to 447Fv and of V3_{IIIB} bound to 0.5β have different conformations. In the 447Fv complex, S313 and R315 form intrapeptide hydrogen bonds. In contrast, in 0.5β complex, the intrapeptide hydrogen bonds are formed by residues I314 and I316 with residues of the C-terminal β-strand. This “one-register” shift in hydrogen bond-forming residues was responsible for an altered topology of side chains in the N-terminal segment. When the N-terminal β-strands of the V3_{IIIB} peptides bound to the two different antibodies are superimposed for best fit, the topological change was manifest as a 180° inversion in the continuation of the C-terminal strand (FIG. 12). In contrast, the C-terminal β-strand, ³²⁴FVTI³²⁷, shares the same conformation in the complex with 447Fv and with 0.5β; residues V325 and I327 form intra-peptide hydrogen bonds with the N-terminal β-strands in both complexes. The topology of the C-terminal β-strand side chain conformation is therefore very similar for the V3_{IIIB} complexes with each of these mAbs.

### The 447-Bound V3_{IIIB} Structure is Homologous to the β-Hairpin in R5 Chemokines

The 0.5β mAb was raised against the gp120 of an X4-type HIV-1_{IIIB} strain. 447-52D is a broadly neutralizing antibody isolated from an HIV-1-infected patient, so the antigen against which it was “induced” is obviously unknown.

Both IIIB and MN strains of HIV-1 are X4 viruses that utilize CXC-R4 as co-receptor. Therefore it was not surprising to find that the V3_{IIIB} peptide bound to the 0.5β mAb was homologous to an X4 chemokine (Sharon et al. (2003) supra and hereinabove). However, it was highly unexpected to find that V3_{MN} peptide when bound to 447-52D was homologous to the structures of R5 chemokines.

It is therefore interesting that when complexed with the 447, the V3_{IIIB} peptide takes on a structure that is homologous to (1) the V3_{MN} bound to the same antibody and (2) a β-hairpin in the R5 chemokines. Superposition of the β-strands in MIP-1β and 447Fv-bound V3_{IIIB} revealed an rmsd of 1.32 Å when the segments ⁴¹VFQ⁴³ and ⁴⁸QVCA⁵¹ of MIP-1β were superimposed over the segments ³¹⁴IRI³¹⁶ and ³²⁴FVTI³²⁷ [SEQ ID NO:43] of V3_{IIIB}. In contrast, when V3_{MN} sequences ³¹⁴IHI³¹⁶ and ³²⁴FYTT³²⁷/³²⁴FVTI³²⁷ [SEQ ID NO:44]/[SEQ ID NO:43] are superimposed on relevant parts of the MIP-1β sequence, an rmsd of only 2.23 Å was noted. Thus, V3_{IIIB} bound to 447 shows even greater likeness to R5 ligands.

This result may be understood by comparing the C-terminal β-strands of the two 447Fv-bound peptides and chemokines of the two classes (R5 and X4). The sequence and conformation of the C-terminal β-strand is conserved between the R5 ligands (MIP-1α, MIP-1β, RANTES) and the X4 ligand, SDF-1. In all four chemokines V49 (V50 in MIP-1α) and A51/I51 (A52 in MIP-1α) are the residues forming hydrogen bonds with the N-terminal β-strand of the β-hairpin. In the conformationally related C-terminal β-strand of V3_{IIIB} bound to 447, V325 and I327 occupy positions that are homologous to chemokine residues V49 and A51/I51. In keeping with this, V325 and I327 form hydrogen bonds with the N-terminal β-strand within the V3 loop.

The C-terminal β-strand of V3_{MN} differs in its hydrogen bonding pattern from V3_{IIIB} and the R5 chemokines. In contradistinction, pattern of the C-terminal β-strands of 447-bound V3_{IIIB} resembles that of the R5 chemokines. In addition, the same N-terminal β-strand hydrogen bonding patterns is observed within 447-bound V3_{MN}, 447-bound V3_{IIIB} and the R5 chemokines.

The difference between the R5 chemokines and SDF-1 lies in the conformation of the N-terminal β-strand. In R5 ligands, V39 (V40 in MIP-1α) and F41 (F42 in MIP-1α) form the hydrogen bonds with the N-terminal β-strand within the β-hairpin as in the V3 β-hairpin. In contrast, in the X4 ligand SDF-1, the hydrogen bonding is formed by residues A40 and L42. As shown above, the N-terminal strands of V3_{IIIB} and V3_{MN} peptides bound to 447 are conformational similar to the R5 ligands while the N-terminal strand of V3_{IIIB} bound to 0.5β shows conformational and sequence similarity to the X4 chemokine SDF-1.

In view of the foregoing, it is apparent that a one-register shift in hydrogen-bond forming residues in the N-terminal β-strand alone can trigger a switch between R5 and an X4 viral phenotypes. This switch is exemplified by the V3_{IIIB} peptide when it is bound to 447-52D and the V3_{IIIB} peptide when it is bound to 0.5β. Alternatively, a one-register shift in both strands of the β-hairpin may bring about this change, as exemplified by V3_{MN} peptide bound to 447 vs V3_{IIIB} peptide bound to 0.5β. Although neither mechanism may be ruled out, the first alternative (a one-register shift in the N-terminus alone) is the one that describes the relationship between the R5 chemokines and an X4 chemokine.

### Example X

### Analysis of Internally-Constrained V3-Like Peptides

Two internally-constrained V3-like peptides, each with two disulfide bonds were prepared as described in Example I.

### A. X4-M1 Peptide

A peptide having the sequence GCKSICIGPGRACYTTCG [SEQ ID NO:19], and designated X4-M1 was designed to be a mimic of the conformation of the X4 V3 loop (and the chemokine SDF-1). This name reflects the fact that this peptide, albeit based on the sequence of V3_{JRFL} loop of an R5 virus, mimics an X4-type conformation, that of V3_{IIIB} as bound to and constrained by mAb 0.5β. The conformational change induced by this antibody on linear V3 nm peptide converts it from a more flexible stayed to an X4 conformation. X4-M1 includes two specified disulfide bridges formed by four Cys residues substituted into the sequence of V3_{JRFL}. The chemical formula of X4-M1 with disulfide bridges indicated is shown below (aligned with the V3_{JRFL} sequence).

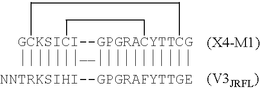

The structure of peptide X4-M1 was solved by NMR and found to be very similar to the conformation of the V3_{IIIB} peptide bound to the 0.5β mAb. The NMR coordinates are presented in Table 5. The RMSD for the residues forming the β-strands was 0.7 Å between the two structures. The network of hydrogen bonds within the peptide was the same for V3 nm bound to 0.5β and for X4-M1 indicating that the topology of the sidechains is also very similar.

### B. R5A-M1 Peptide

A peptide having the sequence CRKSICH--GPGRCFYTTGC [SEQ ID NO:18] and designated R5A-M1 (mimic #1 of one of two types of R5-binding peptides, R5A) was designed to be a mimic of the R5 V3 loop (and chemokine) conformation. The sequence is based on the sequence of V3_{JRFL} loop of an R5 virus and mimics the structure of V3_{MN} bound to 447. R5A-M1 includes two specified disulfide bridges formed by four Cys residues substituted into the sequence of V3_{JRFL}. The chemical formula of R5A-M1 with disulfide bridges indicated is shown below (aligned with the V3_{JRFL} sequence).

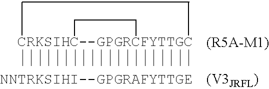

The structure of R5A-M1 has been solved with lower resolution, showing an rmsd of 1.46 Å with the structure of V3_{MN} bound to 447Fv (the R5A conformation). The NMR coordinates are presented in Table 6. Only two of the six hydrogen bonds (between H315 and F324) have been identified in the R5A-M1 structure and they are the same as in the R5A structure.

The R5A-M1 peptide binds 447Fv with a dissociation constant of 10 nM as determined by fluorescence quenching of 447F upon titration with the peptide, so is within one order of magnitude of the binding affinity of the V3 peptide (˜1 nM).

### Peptide Mimics of a Second R5 Conformation

Another new R5 conformation exemplified by V3_{IIIB} bound to 447Fv, and designated R5B (in distinction from R5A which is exemplified by V3MN bound to 447Fv) was discovered by the present inventors, as is discussed in the above can be mimicked by constrained peptides, based on the V3_{JRFL} sequence. The requisite pattern of hydrogen bonds can be achieved in several ways. One preferred embodiment utilizes two disulfide bonds as shown in the formula for R5B-M1 below (with an aligned V3_{JRFL} sequence).

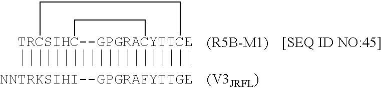

In another embodiment, the hydrogen bonding is achieved by a single disulfide bond—as is shown below in another sequence—that is designated R5B-M2. (Also shown is an aligned V3_{JRFL} sequence.)

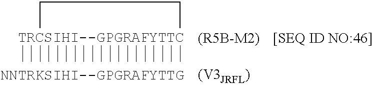

In the case where Cys replaces an Ile, as in R5A-M1 and R5B-M1, one may alternatively replace the Ile with penicillamine. Penicillamine has two methyl groups on the β-carbon, thus resembling Ile even more closely, and also has an —SH group on the β carbon that can form a disulfide bond and constrain/cyclize the peptide.

Thus, the two conformations recognized by 447-52D can be restricted by chemical means. However, the synthesis of constrained peptide analogs will require modifications in the location of the constraining elements within the peptide used as immunogens or as R5 and XR4 antagonists.

The above structures, including X4-M1 and R5A-M1. R5B-M1/M2 show how one can mimic three different conformations recognized by 447-52D or other broadly neutralizing and V3-loop antibodies. These structures exemplify but several ways in which such conformations can be imposed on linear peptides by chemical means which includes cyclization via one or two disulfide bridges. Other means to achieve this can be readily discerned by one of skill in the art using known chemistries and the guidance presented herein, particularly the included NMR structural coordinates. Thus constrained peptide analogues with the requisite biological activity, to be used as immunogens or as co-receptor antagonists, can be made by the appropriate modifications and introduction of constraining elements into the V3 peptide(s).

### Example XI

### Modeling of Chimeric V3 Mimics

### 1. V3/Chemokine Chimeric Proteins

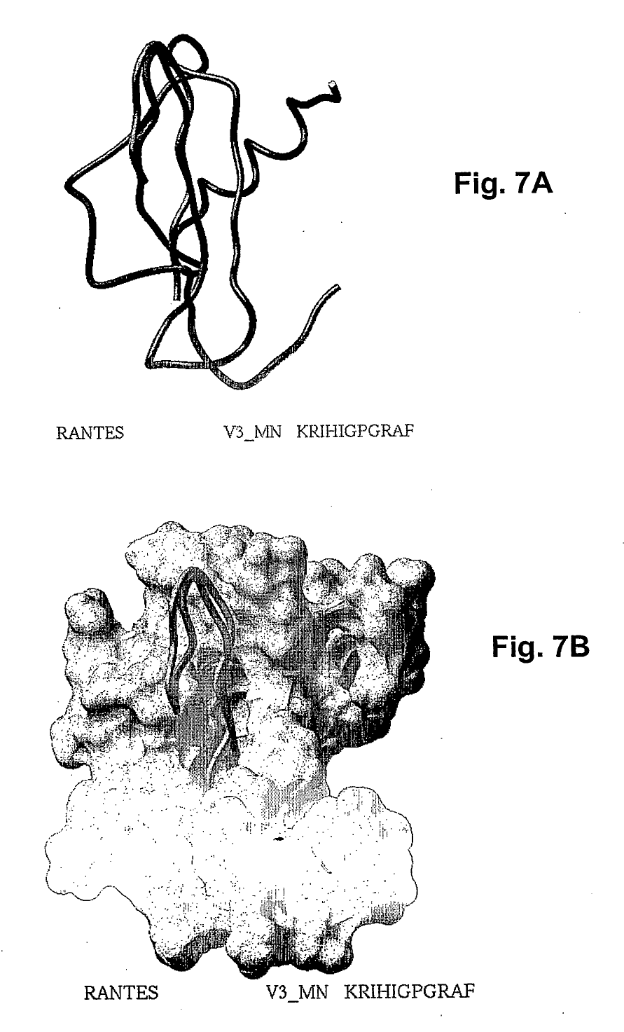

Chimeric immunogens were designed by grafting the V3 loop in place of the β-hairpin of RANTES and other ligands of the chemokine receptors R5 and R4. The chemokines, however, appeared not to be the best structural templates for building V3 chimeric or hybrid proteins because clashes appeared when the chemokine “scaffolds” were docked with mAb 447. A structural superposition of the backbone of V3_{MN} truncated to IHIGPGRA [SEQ ID NO:47] (as described above) on a database of all available protein structures identified RANTES only in the top 20% closest structures (pdb code 1rto=hit # 6026 out of 29928). Another structure of RANTES (pdb code 1hrj) was in the top 40%. Adding the sequence filter IxxGPGxxxYxT [SEQ ID NO:29] identified RANTES as the 17^{th} best of 29928 sequences (top 0.050%). The results of this search appear in Table 7. Forty five structures closest to V3_{MN} matched the above sequence filter pattern. Note that the structural superposition renders less important the amino acid residue in a particular location. RANTES=hit#17; MIP1-α=hit #29. Superposition of V3_{MN} and RANTES are shown in FIGS. 7A and B.

The next approach taken was to search the protein data base (PDB) for molecules containing β-hairpins whose size and shape could optimally be superimposed on the structure of the relevant V3 in its form when it is bound to a neutralizing mAb such as 447. The interposition of different “filters” constructed of various amino acid motifs responsible for critical interactions between V3 and mAb 447 reduced the number of “hits” and focused the search on relevant structures and/or amino acid motifs.

After determining the closeness of fit between the relevant V3 and the selected homologues, models were built of chimeric molecules in which the relevant V3 motif was grafted onto the selected scaffold. Two such “V3 chimeric mimetic immunogens” were designed.

### 2. Chimeric V3/α-Defensin

α-defensin was identified as a potential scaffold for a V3 immunogen on the basis of the size and shape of its β-hairpin region and the presence within the hairpin of an amino acid motif resembling that of the V3 loop. Structural studies suggest that the tip of the β-hairpin of β-defensin can be replaced with critical residues of the V3 loop, giving rise to a chimeric structure which docks with the broadly neutralizing mAb 447.

This came about from a search performed similarly to that above using as a filter (or consensus sequence) the sequence xIxxGRxx [SEQ ID NO:48]. Search results shown in Table 8, revealed that defensin-α (or α-defensin or defensin-1), a low molecular weight granulocyte protein, superimposed well with V3_{MN}. This is shown below, with α-defensin identified by its pdb code as 1dfn_a, compared to V3_{MN} and V3_{IIIB}. The stretches of E's on the bottom line of each grouping represents β-strand structure.

| | | |
|---|---|---|
| Consensus | ...~I~.GR##. | |
| v3_nm | ---------------KRIHIGPGRAFYTT--- | SEQ ID NO:20 |
| 1dfn_a | DCYCRIPACIAGERRYGTCIYQGRLWA-FCC | SEQ ID NO:49 |
| 1dfn_a | EEE EEEEEEEE EEEE EEE | |
| Consensus | .....~...# | |
| 1dfn_a | DCYCRIPACIAGERRYGTCIYQ--GRLWAFCC | SEQ ID NO:49 |
| V3_IIIb | -----------------IRIQRGPGRA----- | SEQ ID NO:51 |
| #1dfn_a | EEE EEEEEEEE EEEEEEE | |

Based on the “alignment” between defensin and V3_{MN}, all the sequence of all β-hairpin structures with backbone rmsd less than 1.5 Å apart from V3_{MN} (as disclosed herein) were d searched for the pattern (I/T/L/V/A)xIxxG(R/K)(T/A/L/V/I) [SEQ ID NO:52]. The hits are shown in Table 8.

Urease was identified as hits #2 and #3. It is interesting that H. pylori urease seems to correlate with HIV infection in that exposure to urease may trigger immunogenic response against V3, or, conversely, V3 may trigger an anti urease response, which might explain the repeated occurrence of urease-negative strains of H. pylori in HIV-1-infected patients. The alignment between V3_{MN} and urease (pdb code 1e9y) is very good.

| | | |
|---|---|---|
| Consensus | ................I.I..G+#.. | |
| V3_mn | ----------------IHIGPGRA-- | SEQ ID NO:47 |
| 1e9y_b | ITNALIVDYTGIYKADIGIKDGKIAG | SEQ ID NO:50 |

Hit #4, pap-specific phosphatase (pdb cod 1flf) is a tumor specific T cell antigen and shows good alignment:

| | | |
|---|---|---|
| V3_mn | ----------------IHIGPGRA---------- | SEQ ID |
| | | NO:47 |
| 1f1f_a | DGTLGFVRGDQYAVALALIENGKVLLGVLGCPNY | SEQ ID |
| | | NO:53 |
| 1f1f_a | HHHHHHH EEEEEE EEEEEE | |

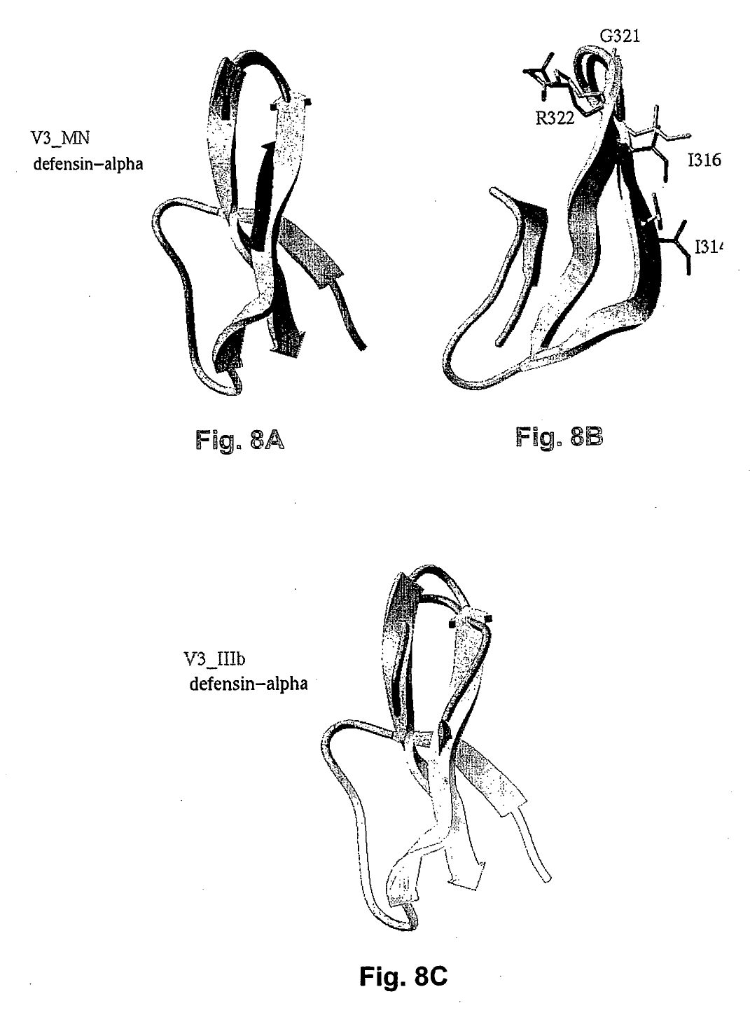

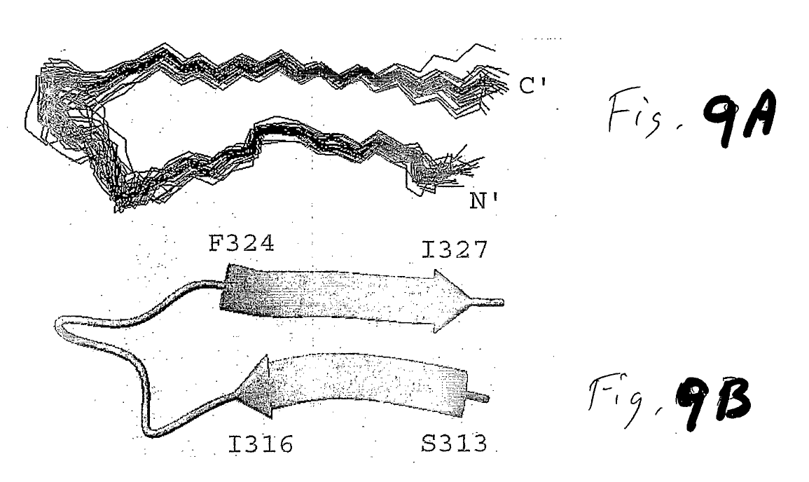

Ho's group proposed that α-defensin (defensin-1) had anti-HIV activity (Zhang et al., 2002, Science 298:995-1000). This activity was observed against both R5 and X4 tropic virus strains. The present inventors' modeling studies found that the hairpin turn of α-defensin superimposed well with the homologous regions of V3 loops that bind to mAbs that neutralize both R5 and X4 viruses. Further modeling was undertaken to optimize the shape and energy minimization of the chimera's loop region. The chimeric V3_{MN}/α-defensin structure, is shown in FIGS. 8A and 8B. The chimeric V3_{IIIb}/α-defensin structure is shown in FIG. 8C.

Critical residues are conserved between V3 and α-defensin, suggesting a model wherein the anti-HIV activity of α-defensin is mediated by competition for binding to chemokine receptors: the α-defensin β-hairpin competes with the V3 loop of HIV-1 virions. These results support the use of a chimeric α-defensin in which a V3_{MN} or V3_{IIIB} peptide has replaced the native segment, resulting in a well-constrained V3 region that is (1) optimized for inducing neutralizing antibodies or (2) an even better competitive binding inhibitor at R5 or X4 receptors.

For use as an immunogen, because of the small size of the defensins, the chimeric α-defensin/V3 polypeptide is conjugated to an immunogenic carrier or fused to an immunogenic carrier, preferably a protein, as is conventional in the immunology art.

The chimeric immunogen is used in two ways. First, it can serve as an inducer of a primary immune response that may be protective in an uninfected subject. Second, it may be used as a booster, either in an uninfected or in an infected or previously immunized subject, to focus the immune response toward a conformationally relevant form of V3. This will result in broadly reactive, highly potent HIV-neutralizing antibodies.

### 3. Chimeric V3/Bowman-Birk Inhibitor

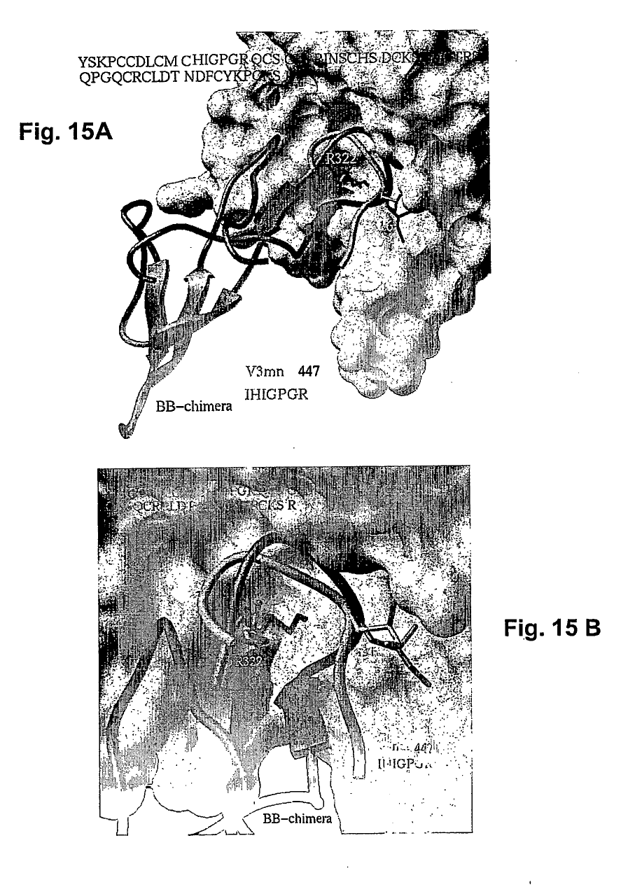

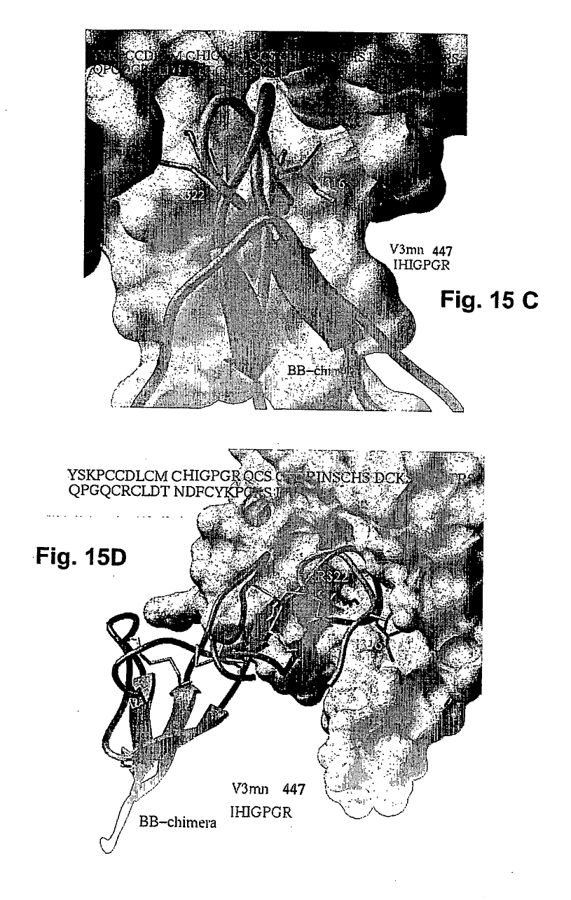

The Bowman-Birk trypsin inhibitor (BBI) derived from soy beans was identified in a search of the PDB for proteins that (a) superimposed with the X-ray structure of the V3_{MN} loop bound to mAb 447, and (b) did not display a steric clash when docked with the X-ray-derived structure of V3-bound 447. Subsequent modeling studies suggested that the tip of the BBI β-hairpin could be replaced with the critical HIGPGR [SEQ ID NO:54] residues of the V3 loop, giving rise to a chimeric structure which docked optimally with the broadly neutralizing mAb 447. See FIG. 15A-15D. Energy minimization of the chimera's loop region produces a structure where critical residues such as the Ile and Arg residues of the IGPGR motif in the chimeric V3/BBI made contacts similar to those observed in the crystal structure of the V3_{MN}/447 complex. No steric clash was observed between mAb 447 and the BB/V3 chimera. All disulfide bridges of the template were conserved.

BBI was selected based on it structural homology to 447-complexed V3_{MN}, and the absence of predicted steric clash between 447 and a V3/BBI chimera. Listed below are a consensus sequence [SEQ ID NO:55] the relevant BB sequence [SEQ ID NO:56] and the sequence of the V3/BBI chimera [SEQ ID NO:57] with the V3-derived residues underscored.

Consensus
YSKPCCDLCMC~.\hat{ }#..QCSCEDRINSCHSDCKSCMCTRSQPGQCRCLDT
NDFCYKPCKSR
BB
YSKPCCDLCMCTRSMPPQCSCEDRINSCHSDCKSCMCTRSQPGQCRCLDT
NDFCYKPCKSR
BB/V3
YSKPCCDLCMCHIGPGRQCSCEDRINSCHSDCKSCMCTRSQPGQCRCLDT
NDFCYKPCKSR

One advantage of the BB/V3 chimeric polypeptides is that because of the larger size of BB, there may be no need of conjugation to increase immunogenicity. Another advantage is that BB is already being administered to humans as a potential cancer therapeutic (Wan, X S et al. (2002) Nutr Cancer. 43:167-173) and various aspects of its pharmacodynamics and lack of toxicity are known.

### Production and Expression of Chimeric Polypeptides

Nucleic acids for expressing these molecules are synthesized using known methods and may be obtained commercially (e.g., from GeneArt, Inc. or GenScript, Inc). The nucleic acid molecule is cloned into standard plasmid vectors (pUC19, Topo vector) or into an expression vectors of the customer's choice. The cloning, expression in E. coli, and purification strategies for small His-tagged proteins are described in (Piers, K L et al., 1993. Gene 134:7; Fang, X L et al., 2002, Protein Pept Lett 9:31). A Met residue can be introduced just before the first amino acid of each protein to facilitate cleavage of the His-tag and of any extra amino acids using CNBr. The purity and the integrity of the purified His-tagged recombinant chimeric proteins are assessed by silver staining of gels, protein sequencing, and by reactivity with anti-His antibody (Novagen), and/or with anti-human α-defensin antibody (Alpha Diagnostic International, Inc.) on Western blots. The His-tag and the extra amino acids, including Met are removed by CNBr treatment.

Those chimeric polypeptides that are too small to be optimally immunogenic are conjugated to tetanus toxoid by standard methods (e.g., Beenhouwer, D et al., 2002, J. Immunol 169:699) to enhance their immunogenicity.

### Example XII

### Testing of Chimeric V3 Immunogens

MAbs with broad and potent neutralizing activity can act as a template for identifying and designing immunogens that will induce broad and potent polyclonal neutralizing antibodies in a subject who is to be immunized or otherwise treated in accordance with this invention. Such immunogens will focus the immune response on epitopes known to be targets of neutralizing antibodies. Immunization of HIV-negative volunteers with either gp120 or a prime/boost regimen such as recombinant canarypox and gp120 is known to induce antibodies to many epitopes of gp120; however potent antibodies that neutralize a broad array of HIV-1 primary isolates have not been produced.

One means of focusing the immune response on broadly neutralizing epitopes of gp120 is to induce memory by priming against whole gp120 and boosting using a construct that would focus the immune response on a broadly neutralizing epitope. Such a prime/boost strategy has been used successfully by Beenhouwer et al. supra, to induce protective antibodies against Cryptococcus neoformans, where the boost was a peptide mimotope identified by screening a protective mAb to C. neoformans with a phage display library.

Since the V3 loop is highly immunogenic and certain mAbs antibodies to V3 can have broad neutralizing activity, it is advantageous to focus the immune response to this epitope, and, eventually, to other neutralizing epitopes. To do this, a boost containing a relevant and immunogenic form of the neutralizing epitope is necessary. Several studies have investigated the utility of various linear or cyclic V3 peptides as immunogens, although none of these has been used in a prime/boost regimen. The present inventors and others have shown in animals that both linear and cyclic V3 peptides given as the sole immunogen induce antibodies with neutralizing activity against homologous and heterologous TCLA strains of HIV Cabezas et al., supra; Conley et al., supra). Other studies showed that longer V3 peptides are more immunogenic than shorter ones, perhaps because the former can be partially stabilized by the formation of a β-turn around the GPGR tip. However, in general, both linear and cyclic peptides are conformationally heterogeneous in aqueous solution, differing from the structures of the cognate sequences in the parent protein and giving rise to anti-peptide antibodies that are incompatible with native protein surfaces (Stanfield, R L et al. (1990) Science 248:712).

The use of a relatively stable and conformationally correct V3 loop peptides or mimics (V3 mimotopes) as described herein as a boosting agent should induce antibodies with broader and more potent neutralizing activity. This expectation is supported by the present inventors' earlier studies of selection of anti-V3 mAbs with V3_{JR-CSF}-Fusion Protein (V3-FP). This is a fusion protein constructed from a truncated form of MuLV gp70 and the V3 sequence from a clade B HIV-1 virus, JR-CSF (derived from the cerebrospinal fluid of patient JR). See, for example, Gorny et al., 2002, supra. Use of V3-FPs possessing conformationally correct V3 loops resulted in mAbs with greater neutralizing activity than did screening with linear V3 peptides.

V3 mimetic immunogens are designed and produce as described herein based on the present inventors' NMR, crystallographic, and protein modeling studies of V3 peptides bound to broadly neutralizing human anti-V3 mAbs such as 447. In one embodiment, these mimetic immunogens are to used as boosts in subjects (which may be experimental animals) primed with, for example, a gp120 DNA vaccine. The antibody activity in the sera of these subjects is compared with that in the sera of other subject who are boosted with carrier-conjugated linear V3 peptides, V3-FP, and/or gp120.

These studies are done with an existing gp120 DNA vaccine based on clade A strain CA1 and employ as a “positive control,” the V3-FP noted above. As clade C neutralizing anti-V3 mAbs become available and the structure of the clade C V3 loop is elucidated through analyses as are described herein additional gp120 DNA vaccine constructs and V3-FPs are made and used to induce and focus the antibody response to generate neutralizing antibodies borne by primary isolates from clades A, B, C and/or by viruses of the various “neutrotypes.”

### Test V3 Mimetic Immunogens as Boosters.

Some studies will utilize V3_{JR-CSF}-FP as booster because these molecules are known to possess biologically relevant V3 conformations. Priming will be done with the gp120 plasmids containing the clade A envelope (CA1), and control boosting will employ V3-FP containing the V3_{JR-CSF}. This protocol is designed to induce cross-reactive anti-V3 antibodies (Gorny et al., supra). This protocol tests the relative efficiencies of the immunogens of the present invention with V3-FP_{JR-CSF} V3_{JR-CSF} linear peptide (conjugated to tetanus toxoid, it), and gp120_{JR-FL} (with and without priming with gp120 DNA). This strategy is based on the classic “carrier effect” of Ovary and Benacerraf which showed that priming with both a haptenic epitope (in this case the V3 loop) and a carrier (in this case MuLV gp70 or a hybrid protein of the present invention) was necessary for an optimal secondary response to the hapten-carrier used as the booster (in this case V3 peptide grafted to α-defensin or BBI (see below) ± conjugated tetanus toxoid. Serially collected sera from all subject are studied by binding assay (ELISA) followed by neutralization assays.

a. Chimeric V3/α-defensin.

As noted above, α-defensin was identified as a potential scaffold for a V3 mimetic immunogen. The chimeric V3/α-defensin will be prepared as described above and will initially be tested for its antigenic reactivity in ELISA experiments. The affinity of mAbs 447 and 2182 (currently the most cross-reactive of the present inventors' anti-V3 mAbs) for the chimeric molecule will be examined and compared to their affinities for V3/_{JR-CSF}-FP and other fusion proteins

If the affinity of the chimeric V3/α-defensin with either of these mAbs is within one order of magnitude of that for either of the mAbs for the V3-FPs, then the chimeric V3/α-defensin will be conjugated to tetanus toxoid according to standard techniques and used in vivo to boost gp120 DNA-primed subjects.

Conjugation is preferred because of the relatively small size of this chimeric molecule (30 amino acids); priming with the tetanus toxoid carrier may also enhance the quality and quantity of anti-V3 antibodies due to the carrier effect described above.

As described above, 20 subjects are used per group—these can be rabbits in preliminary studies. The protocol tests the relative efficiencies of V3-FP_{JR-CSF}, V3/α-defensin and gp120 of JR-FL, with and without priming with gp120 DNA to focus the antibody response on the V3 loop and induce neutralizing antibodies. The use of tt in the priming regimen in one control and one experimental group is based on the carrier effect which may indicate that priming with both the haptenic epitope (in this case, the V3 loop which is included in the gp120 priming regimen) and the carrier (in this case, tt) is preferred for an optimal secondary response to the hapten-carrier used as the booster (in this case V3/α-defensin/tt or V3/BBI/tt). One advantage of using tetanus toxoid is that it is used extensively in humans, and so “priming” with this will have already occurred in most subjects. Serially collected sera are first analyzed by ELISA, followed by neutralization assays. Expected results are shown in Table 9

c. Chimeric V3/Bowman-Birk Inhibitor (BBI)

As described above, BBI was identified as a potential scaffold for a chimeric molecule with a “grafted” V3 sequence which docks optimally with the broadly neutralizing mAb 447.

Its ability to react immunologically with anti-V3 mAbs is tested as described above for the V3/α-defensin chimeras. If the affinity of a chimeric V3/BBI with mAbs 447 (or other broadly neutralizing mAbs) is within one log of the affinities of these mAbs for the V3-FPs, the chimeric V3/BBI molecule will be used as a V3 mimetic booster in gp120 DNA-primed subjects. The chimeric V3/BBI is prepared as described above and conjugated to tetanus toxoid (to increase the immunogenicity of this molecule which has only 61 amino acids). A preferred immunization protocol, shown in Table 10, tests the relative efficiencies of V3-FP_{JR-CSF}, V3/BBI-tt and gp120 of JR-FL, with and without priming with gp120 DNA to focus the antibody response on the V3 loop and induce neutralizing antibodies. Numbers of subjects are as in the study above. Serially collected sera from all immunized subjects are tested by ELISA and neutralization assays.

Advantages of the V3/BBI over the V3/α-defensin chimera include the fact that (a) V3/BBI seems to accommodate to the 447 binding site structure with no steric clashes, (b) no auto-immune responses are expected, (c) all disulfide bridges are conserved, and (d) BBI has already been used in humans.

An increase in the quality and quantity of neutralizing activity after immunization with a chimeric V3/α-defensin or a V3/BBI chimeric molecule compared to that induced by the appropriate control would indicate that that the chimeric immunogen has the desired mimetic conformation and is useful for the induction of preventative or therapeutic antibody response in a subject. Increased “quality” of the response would be measured by the neutralization of significantly more primary HIV-1 isolates. An increased quantitative response would be defined by a statistically significant increase in the neutralizing titer of the sera. As noted above, HIV-1 neutralization assays are well known in the art (Mascola et al., 2002, supra; Montefiori et al., supra; D'Souza et al., supra).

Tables 1-10 are shown below.

All the references cited in this document are incorporated herein by reference in their entirety, whether specifically incorporated or not.

Having now fully described this invention, it will be appreciated by those skilled in the art that the same can be performed within a wide range of equivalent parameters, concentrations, and conditions without departing from the spirit and scope of the invention and without undue experimentation.

TABLE 1
NMR constraints and structural statistics for the
refined 447Fv-bound V3_{MN} peptide (29 structures)
NMR distance constraints

| | |
|---|---|
| Total constraints | 305 |
| Intra-residue | 155 |
| Sequential | 60 |
| Medium and long range | 90 |
| Dihedral angles | 10 |

NOE violations

| | |
|---|---|
| Maximum individual violation (Å) | 0.5 |
| Rmsd of NOE violation | 0.0012 ± 0.0026 |

Deviation from ideal covalent geometry

| | |
|---|---|
| Bond lengths (Å) | 0.0015 Å ± 0.0002 Å° |
| Bond angles (°) | 0.4109° ± 0.0105° |
| Improper angles (°) | 0.1394° ± 0.0183° |

Mean rmsd values (Å)

| | |
|---|---|
| All backbone atoms | 0.37 Å |
| All heavy atoms | 1.17 Å |

TABLE 2
NMR constraints and structural statistics for the refined structures
of the 447Fv-bound V3_{IIIB} peptide (29 structures)
NMR distance constraints

| | |
|---|---|
| Total constraints | 365 |
| Intra-residue | 220 |
| Sequential | 55 |
| Medium- and long-range | 75 |
| Dihedral angle | 21 |

NOE violations

| | |
|---|---|
| Maximum individual violation (Å) | 0.4 Å |
| Rmsd of NOE violation | 0.0090 ± 0.0023 |

Deviation from ideal covalent geometry

| | |
|---|---|
| Bond lengths (Å) | 0.0011 Å ± 0.0001 Å |
| Bond angles (°) | 0.3716° ± 0.0077° |
| Improper angles (°) | 0.1106° ± 0.0130° |

Mean rmsd values (Å)

| | |
|---|---|
| All backbone atoms | 0.58 Å |
| All heavy atoms | 1.31 Å |

TABLE 3

Average NMR Coordinates of V3_{MN}-Constrained
by 447-52D Fv
³¹²KRIHI--GPGRAFYTT³²⁷ [SEQ ID NO:20]

| | | | | | | |
|---|---|---|---|---|---|---|
| A | B | C | D | X | Y | Z |

| | | | | | | |
|---|---|---|---|---|---|---|
| 1 | N | Lys | 312 | 6.901 | −3.254 | −4.724 |
| 2 | HN | Lys | 312 | 7.275 | −2.763 | −5.484 |
| 3 | Cα | Lys | 312 | 6.188 | −2.514 | −3.698 |
| 4 | Hα | Lys | 312 | 6.161 | −3.119 | −2.804 |
| 5 | Cβ | Lys | 312 | 6.934 | −1.209 | −3.400 |
| 6 | Hβ1 | Lys | 312 | 7.416 | −0.877 | −4.306 |
| 7 | Hβ2 | Lys | 312 | 7.686 | −1.402 | −2.652 |
| 8 | Cγ | Lys | 312 | 6.049 | −0.083 | −2.900 |
| 9 | Hγ1 | Lys | 312 | 5.540 | −0.407 | −2.005 |
| 10 | Hγ2 | Lys | 312 | 5.325 | 0.151 | −3.665 |
| 11 | Cδ | Lys | 312 | 6.858 | 1.165 | −2.588 |
| 12 | Hδ1 | Lys | 312 | 6.183 | 1.996 | −2.460 |
| 13 | Hδ2 | Lys | 312 | 7.526 | 1.364 | −3.413 |
| 14 | Cε | Lys | 312 | 7.679 | 0.994 | −1.320 |
| 15 | Hε1 | Lys | 312 | 7.930 | −0.051 | −1.207 |
| 16 | Hε2 | Lys | 312 | 7.086 | 1.314 | −0.477 |
| 17 | NZ | Lys | 312 | 8.935 | 1.793 | −1.360 |
| 18 | HZ1 | Lys | 312 | 9.236 | 2.034 | −0.394 |
| 19 | HZ2 | Lys | 312 | 8.783 | 2.672 | −1.893 |
| 20 | HZ3 | Lys | 312 | 9.691 | 1.248 | −1.822 |
| 21 | C | Lys | 312 | 4.762 | −2.222 | −4.146 |
| 22 | O | Lys | 312 | 4.448 | −2.291 | −5.335 |
| 23 | N | Arg | 313 | 3.904 | −1.886 | −3.192 |
| 24 | HN | Arg | 313 | 4.212 | −1.840 | −2.263 |
| 25 | Cα | Arg | 313 | 2.518 | −1.573 | −3.500 |
| 26 | Hα | Arg | 313 | 2.507 | −1.070 | −4.454 |
| 27 | Cβ | Arg | 313 | 1.691 | −2.856 | −3.602 |
| 28 | Hβ1 | Arg | 313 | 0.697 | −2.663 | −3.216 |
| 29 | Hβ2 | Arg | 313 | 2.164 | −3.625 | −3.002 |
| 30 | Cγ | Arg | 313 | 1.556 | −3.380 | −5.023 |
| 31 | Hγ1 | Arg | 313 | 2.473 | −3.882 | −5.300 |
| 32 | Hγ2 | Arg | 313 | 1.381 | −2.545 | −5.688 |
| 33 | Cδ | Arg | 313 | 0.400 | −4.359 | −5.147 |
| 34 | Hδ1 | Arg | 313 | −0.085 | −4.205 | −6.100 |
| 35 | Hδ2 | Arg | 313 | −0.304 | −4.167 | −4.351 |
| 36 | NE | Arg | 313 | 0.846 | −5.747 | −5.059 |
| 37 | HE | Arg | 313 | 1.796 | −5.927 | −5.218 |
| 38 | CZ | Arg | 313 | 0.041 | −6.769 | −4.778 |
| 39 | NH1 | Arg | 313 | −1.252 | −6.563 | −4.558 |
| 40 | HH11 | Arg | 313 | −1.627 | −5.637 | −4.602 |
| 41 | HH12 | Arg | 313 | −1.851 | −7.335 | −4.347 |
| 42 | NH2 | Arg | 313 | 0.529 | −8.000 | −4.719 |
| 43 | HH21 | Arg | 313 | 1.502 | −8.161 | −4.884 |
| 44 | HH22 | Arg | 313 | −0.076 | −8.768 | −4.507 |
| 45 | C | Arg | 313 | 1.921 | −0.646 | −2.446 |
| 46 | O | Arg | 313 | 1.783 | −1.021 | −1.282 |
| 47 | N | Ile | 314 | 1.569 | 0.565 | −2.864 |
| 48 | HN | Ile | 314 | 1.705 | 0.805 | −3.804 |
| 49 | Cα | Ile | 314 | 0.987 | 1.548 | −1.957 |
| 50 | Hα | Ile | 314 | 0.975 | 1.120 | −0.964 |
| 51 | Cβ | Ile | 314 | 1.818 | 2.846 | −1.916 |
| 52 | Hβ | Ile | 314 | 1.571 | 3.431 | −2.788 |
| 53 | Cγ1 | Ile | 314 | 3.316 | 2.527 | −1.935 |
| 54 | Hγ11 | lIe | 314 | 3.458 | 1.480 | −1.695 |
| 55 | Hγ12 | Ile | 314 | 3.820 | 3.139 | −1.196 |
| 56 | Cγ2 | Ile | 314 | 1.459 | 3.664 | −0.684 |
| 57 | Hγ21 | Ile | 314 | 0.689 | 4.377 | −0.938 |
| 58 | Hγ22 | Ile | 314 | 2.335 | 4.190 | −0.334 |
| 59 | Hγ23 | Ile | 314 | 1.099 | 3.006 | 0.093 |
| 60 | Cδ1 | lIe | 314 | 3.969 | 2.790 | −3.275 |
| 61 | Hδ11 | lIe | 314 | 3.292 | 3.350 | −3.902 |
| 62 | Hδ12 | Ile | 314 | 4.206 | 1.849 | −3.750 |
| 63 | Hδ13 | Ile | 314 | 4.376 | 3.358 | −3.128 |
| 64 | C | Ile | 314 | −0.442 | 1.889 | −2.365 |
| 65 | O | Ile | 314 | −0.696 | 2.275 | −3.506 |
| 66 | N | His | 315 | −1.373 | 1.743 | −1.427 |
| 67 | HN | His | 315 | −1.109 | 1.432 | −0.537 |
| 68 | Cα | His | 315 | −2.776 | 2.037 | −1.693 |
| 69 | Hα | His | 315 | −2.821 | 2.703 | −2.541 |
| 70 | Cβ | His | 315 | −3.532 | 0.751 | −2.031 |
| 71 | Hβ1 | His | 315 | −4.580 | 0.985 | −2.176 |
| 72 | Hβ2 | His | 315 | −3.428 | 0.052 | −1.210 |
| 73 | Cγ | His | 315 | −3.034 | 0.076 | −3.272 |
| 74 | Nδ1 | His | 315 | −3.795 | −0.055 | −4.415 |
| 75 | Hδ1 | His | 315 | −4.713 | 0.269 | −4.532 |
| 76 | Cδ2 | His | 315 | −1.843 | −0.508 | −3.546 |
| 77 | Hδ2 | His | 315 | −1.000 | −0.590 | −2.875 |
| 78 | Cε1 | His | 315 | −3.094 | −0.691 | −5.338 |
| 79 | Hε1 | His | 315 | −3.435 | −0.934 | −6.333 |
| 80 | Nε2 | His | 315 | −1.907 | −0.975 | −4.836 |
| 81 | Hε2 | His | 315 | −1.159 | −1.360 | −5.339 |
| 82 | C | His | 315 | −3.425 | 2.722 | −0.494 |
| 83 | O | His | 315 | −3.353 | 2.227 | 0.630 |
| 84 | N | Ile | 316 | −4.058 | 3.864 | −0.743 |
| 85 | HN | Ile | 316 | −4.080 | 4.208 | −1.660 |
| 86 | Cα | lIe | 316 | −4.718. | 4.617 | 0.316 |
| 87 | Hα | Ile | 316 | −4.845 | 3.960 | 1.163 |
| 88 | Cβ | Ile | 316 | −3.869 | 5.823 | 0.762 |
| 89 | Hβ | Ile | 316 | −3.875 | 6.553 | −0.033 |
| 90 | Cγ1 | Ile | 316 | −2.427 | 5.388 | 1.030 |
| 91 | Hγ11 | Ile | 316 | −2.435 | 4.439 | 1.550 |
| 92 | Hγ12 | Ile | 316 | −1.942 | 6.131 | 1.647 |
| 93 | Cγ2 | Ile | 316 | −4.473 | 6.468 | 2.000 |
| 94 | Hγ21 | Ile | 316 | −4.373 | 5.797 | 2.840 |
| 95 | Hγ22 | Ile | 316 | −5.519 | 6.673 | 1.825 |
| 96 | Hγ23 | Ile | 316 | −3.955 | 7.391 | 2.214 |
| 97 | Cδ1 | Ile | 3.16 | −1.603 | 5.222 | −0.229 |
| 98 | Hδ11 | Ile | 316 | −1.191 | 4.224 | −0.260 |
| 99 | Hδ12 | Ile | 316 | −0.800 | 5.944 | −0.230 |
| 100 | Hδ13 | Ile | 316 | −2.231 | 5.379 | −1.093 |
| 101 | C | Ile | 316 | −6.088 | 5.115 | −0.134 |
| 102 | O | Ile | 316 | −6.191 | 6.077 | −0.895 |
| 103 | N | Gly | 319 | −7.137 | 4.454 | 0.343 |
| 104 | HN | Gly | 319 | −6.993 | 3.695 | 0.947 |
| 105 | Cα | Gly | 319 | −8.488 | 4.843 | −0.018 |
| 106 | Hα1 | Gly | 319 | −9.038 | 3.959 | −0.312 |
| 107 | Hα2 | Gly | 319 | −8.443 | 5.526 | −0.855 |
| 108 | C | Gly | 319 | −9.220 | 5.518 | 1.127 |
| 109 | O | Gly | 319 | −8.609 | 6.245 | 1.911 |
| 110 | N | Pro | 320 | −10.539 | 5.292 | 1.256 |
| 111 | Cα | Pro | 320 | −11.336 | 5.891 | 2.329 |
| 112 | Hα | Pro | 320 | −11.187 | 6.958 | 2.382 |
| 113 | Cβ | Pro | 320 | −12.776 | 5.592 | 1.912 |
| 114 | Hβ1 | Pro | 320 | −13.171 | 6.428 | 1.355 |
| 115 | Hβ2 | Pro | 320 | 13.380 | 5.417 | 2.793 |
| 116 | Cγ | Pro | 320 | −12.686 | 4.371 | 1.063 |
| 117 | Hγ1 | Pro | 320 | −13.483 | 4.373 | 0.334 |
| 118 | Hγ2 | Pro | 320 | −12.747 | 3.488 | 1.681 |
| 119 | Cδ | Pro | 320 | −11.350 | 4.435 | 0.372 |
| 120 | Hδ1 | Pro | 320 | −11.454 | 4.880 | −0.607 |
| 121 | Hδ2 | Pro | 320 | −10.920 | 3.447 | 0.293 |
| 122 | C | Pro | 320 | −11.024 | 5.270 | 3.685 |
| 123 | O | Pro | 320 | −11.873 | 4.617 | 4.292 |
| 124 | N | Gly | 321 | −9.795 | 5.468 | 4.147 |
| 125 | HN | Gly | 321 | −9.159 | 5.989 | 3.614 |
| 126 | Cα | Gly | 321 | −9.383 | 4.911 | 5.420 |
| 127 | Hα1 | Gly | 321 | −10.231 | 4.896 | 6.089 |
| 128 | Hα2 | Gly | 321 | −8.611 | 5.537 | 5.844 |
| 129 | C | Gly | 321 | −8.849 | 3.500 | 5.273 |
| 130 | O | Gly | 321 | −8.886 | 2.711 | 6.217 |
| 131 | N | Arg | 322 | −8.355 | 3.183 | 4.079 |
| 132 | HN | Arg | 322 | −8.356 | 3.857 | 3.367 |
| 133 | Cα | Arg | 322 | −7.813 | 1.859 | 3.798 |
| 134 | Hα | Arg | 322 | −8.113 | 1.204 | 4.602 |
| 135 | Cβ | Arg | 322 | −8.371 | 1.310 | 2.475 |
| 136 | Hβ1 | Arg | 322 | −8.704 | 0.293 | 2.636 |
| 137 | Hβ2 | Arg | 322 | −7.580 | 1.308 | 1.737 |
| 138 | Cγ | Arg | 322 | −9.539 | 2.108 | 1.910 |
| 139 | Hγ1 | Arg | 322 | −9.847 | 1.663 | 0.973 |
| 140 | Hγ2 | Arg | 322 | −9.221 | 3.127 | 1.743 |
| 141 | Cδ | Arg | 322 | −10.722 | 2.116 | 2.863 |
| 142 | Hδ1 | Arg | 322 | −11.238 | 3.061 | 2.764 |
| 143 | Hδ2 | Arg | 322 | −10.355 | 2.008 | 3.875 |
| 144 | Nε | Arg | 322 | −11.659 | 1.031 | 2.581 |
| 145 | Hε | Arg | 322 | −11.508 | 0.502 | 1.771 |
| 146 | CZ | Arg | 322 | −12.696 | 0.727 | 3.358 |
| 147 | NH1 | Arg | 322 | −12.933 | 1.422 | 4.463 |
| 148 | HH11 | Arg | 322 | −12.332 | 2.180 | 4.717 |
| 149 | HH12 | Arg | 322 | −13.714 | 1.189 | 5.043 |
| 150 | NH2 | Arg | 322 | −13.499 | −0.276 | 3.029 |
| 151 | HH21 | Arg | 322 | −13.325 | −0.803 | 2.197 |
| 152 | HH22 | Arg | 322 | −14.277 | −0.506 | 3.612 |
| 153 | C | Arg | 322 | −6.288 | 1.899 | 3.745 |
| 154 | O | Arg | 322 | −5.694 | −1.925 | 2.667 |
| 155 | N | Ala | 323 | −5.660 | 1.907 | 4.916 |
| 156 | HN | Ala | 323 | −6.187 | 1.887 | 5.742 |
| 157 | Cα | Ala | 323 | −4.205 | 1.945 | 5.001 |
| 158 | Hα | Ala | 323 | −3.862 | 2.815 | 4.459 |
| 159 | Cβ | Ala | 323 | −3.764 | 2.086 | 6.450 |
| 160 | Hβ1 | Ala | 323 | −3.740 | 3.132 | 6.719 |
| 161 | Hβ2 | Ala | 323 | −2.778 | 1.661 | 6.569 |
| 162 | Hβ3 | Ala | 323 | −4.461 | 1.565 | 7.090 |
| 163 | C | Ala | 323 | −3.586 | 0.699 | 4.376 |
| 164 | O | Ala | 323 | −3.245 | −0.254 | 5.076 |
| 165 | N | Phe | 324 | −3.446 | 0.714 | 3.055 |
| 166 | HN | Phe | 324 | −3.738 | 1.502 | 2.552 |
| 167 | Cα | Phe | 324 | −2.869 | −0.416 | 2.335 |
| 168 | Hα | Phe | 324 | −2.821 | −1.252 | 3.016 |
| 169 | Cβ | Phe | 324 | −3.755 | −0.794 | 1.146 |
| 170 | Hβ1 | Phe | 324 | −3.174 | −1.371 | 0.442 |
| 171 | Hβ2 | Phe | 324 | −4.103 | 0.108 | 0.664. |
| 172 | Cγ | Phe | 324 | −4.959 | −1.606 | 1.529 |
| 173 | Cδ1 | Phe | 324 | −6.237 | −1.146 | 1.253 |
| 174 | Hδ1 | Phe | 324 | −6.363 | −0.195 | 0.757 |
| 175 | Cδ2 | Phe | 324 | −4.813 | −2.828 | 2.165 |
| 176 | Hδ2 | Phe | 324 | −3.822 | −3.195 | 2.385 |
| 177 | Cε | Phe | 324 | −7.347 | −1.891 | 1.605 |
| 178 | Hε1 | Phe | 324 | −8.338 | −1.521 | 1.385 |
| 179 | Cε2 | Phe | 324 | −5.919 | −3.577 | 2.520 |
| 180 | Hε2 | Phe | 324 | −5.792 | −4.528 | 3.015 |
| 181 | CZ | Phe | 324 | −7.188 | −3.108 | 2.239 |
| 182 | HZ | Phe | 324 | −8.054 | −3.691 | 2.515 |
| 183 | C | Phe | 324 | −1.458 | −0.091 | 1.854 |
| 184 | O | Phe | 324 | −1.105 | −0.357 | 0.705 |
| 185 | N | Tyr | 325 | −0.655 | 0.484 | 2.743 |
| 186 | HN | Tyr | 325 | −0.995 | 0.669 | 3.644 |
| 187 | C═ | Tyr | 325 | 0.718 | 0.844 | 2.414 |
| 188 | Hα | Tyr | 325 | 0.761 | 1.056 | 1.356 |
| 189 | Cβ | Tyr | 325 | 1.137 | 2.096 | 3.191 |
| 190 | Hβ1 | Tyr | 325 | 0.978 | 1.925 | 4.245 |
| 191 | Hβ2 | Tyr | 325 | 0.528 | 2.928 | 2.871 |
| 192 | Cγ | Tyr | 325 | 2.587 | 2.483 | 2.995 |
| 193 | Cδ1 | Tyr | 325 | 2.937 | 3.544 | 2.170 |
| 194 | Hδ1 | Tyr | 325 | 2.158 | 4.095 | 1.664 |
| 195 | Cδ2 | Tyr | 325 | 3.604 | 1.788 | 3.638 |
| 196 | Hδ2 | Tyr | 325 | 3.348 | 0.961 | 4.283 |
| 197 | Cε1 | Tyr | 325 | 4.260 | 3.901 | 1.990 |
| 198 | Hε1 | Tyr | 325 | 4.512 | 4.729 | 1.344 |
| 199 | Cβ2 | Tyr | 325 | 4.929 | 2.139 | 3.463 |
| 200 | Hβ2 | Tyr | 325 | 5.705 | 1.587 | 3.971 |
| 201 | CZ | Tyr | 325 | 5.251 | 3.196 | 2.639 |
| 202 | OH | Tyr | 325 | 6.570 | 3.549 | 2.462 |
| 203 | HH | Tyr | 325 | 6.987 | 3.668 | 3.319 |
| 204 | C | Tyr | 325 | 1.668 | −0.310 | 2.723 |
| 205 | O | Tyr | 325 | 1.870 | −0.667 | 3.884 |
| 206 | N | Thr | 326 | 2.248 | −0.889 | 1.677 |
| 207 | HN | Thr | 326 | 2.047 | −0.560 | 0.776 |
| 208 | Cα | Thr | 326 | 3.176 | −2.003 | 1.837 |
| 209 | Hα | Thr | 326 | 3.534 | −1.991 | 2.855 |
| 210 | Cβ | Thr | 326 | 2.462 | −3.330 | 1.576 |
| 211 | Hβ | Thr | 326 | 3.086 | −4.138 | 1.930 |
| 212 | Oγ1 | Thr | 326 | 2.238 | −3.515. | 0.189 |
| 213 | Hγ1 | Thr | 326 | 2.616 | −4.354 | −0.087 |
| 214 | Cγ2 | Thr | 326 | 1.124 | −3.437 | 2.275 |
| 215 | Hγ21 | Thr | 326 | 0.340 | −3.117 | 1.604 |
| 216 | Hγ22 | Thr | 326 | 1.125 | −2.808 | 3.153 |
| 217 | Hγ23 | Thr | 326 | 0.951 | −4.463 | 2.567 |
| 218 | C | Thr | 326 | 4.367 | −1.859 | 0.895 |
| 219 | O | Thr | 326 | 4.218 | −1.928 | −0.325 |
| 220 | N | Thr | 327 | 5.547 | −1.657 | 1.471 |
| 221 | HN | Thr | 327 | 5.601 | −1.611 | 2.448 |
| 222 | Cα | Thr | 327 | 6.765 | −1.503 | 0.684 |
| 223 | Hα | Thr | 327 | 6.492 | −1.075 | −0.269 |
| 224 | Cβ | Thr | 327 | 7.738 | −0.558 | 1.392 |
| 225 | Hβ | Thr | 327 | 8.497 | −0.247 | 0.690 |
| 226 | Oγ1 | Thr | 327 | 8.375 | −1.213 | 2.474 |
| 227 | Hγ1 | Thr | 327 | 7.713 | −1.511 | 3.103 |
| 228 | Cγ2 | Thr | 327 | 7.073 | 0.688 | 1.937 |
| 229 | Hγ21 | Thr | 327 | 6.013 | 0.649 | 1.733 |
| 230 | Hγ22 | Thr | 327 | 7.499 | 1.560 | 1.463 |
| 231 | Hγ23 | Thr | 327 | 7.231 | 0.744 | 3.004 |
| 232 | C | Thr | 327 | 7.433 | −2.853 | 0.445 |
| 233 | O | Thr | 327 | 7.857 | −3.158 | −0.670 |

Column Headings:
A = atom serial no. in peptide
B = atom type/position in amino acid
C = name of amino acid residue
D = residue number in peptide sequence
X, Y, Z = Orthogonal Coordinates (in Angstroms) for X, Y and Z axes

TABLE 4

Average NMR Coordinates of V3_{IIIB}
Peptide Constrained by 447-52D Fv
³¹²KSIRIQRGPGRAFVTIG³²⁸ [SEQ ID NO:28]

| | | | | | | |
|---|---|---|---|---|---|---|
| A | B | C | D | X | Y | Z |

| | | | | | | |
|---|---|---|---|---|---|---|
| 1 | N | Lys | 312 | 28.208 | 5.289 | −4.449 |
| 2 | HN | Lys | 312 | 27.741 | 4.661 | −5.040 |
| 3 | Cα | Lys | 312 | 28.545 | 4.872 | −3.094 |
| 4 | Hα | Lys | 312 | 29.311 | 5.537 | −2.725 |
| 5 | Cβ | Lys | 312 | 27.318 | 4.975 | −2.186 |
| 6 | Hβ1 | Lys | 312 | 27.634 | 4.863 | −1.159 |
| 7 | Hβ2 | Lys | 312 | 26.634 | 4.176 | −2.433 |
| 8 | Cγ | Lys | 312 | 26.577 | 6.294 | −2.316 |
| 9 | Hγ1 | Lys | 312 | 25.568 | 6.167 | −1.952 |
| 10 | Hγ2 | Lys | 312 | 26.552 | 6.580 | −3.357 |
| 11 | Cδ | Lys | 312 | 27.256 | 7.396 | −1.519 |
| 12 | Hδ1 | Lys | 312 | 27.863 | 7.989 | −2.188 |
| 13 | Hδ2 | Lys | 312 | 27.883 | 6.947 | −0.764 |
| 14 | Cε | Lys | 312 | 26.240 | 8.301 | −0.841 |
| 15 | Hε1 | Lys | 312 | 25.993 | 7.883 | 0.124 |
| 16 | Hε2 | Lys | 312 | 25.350 | 8.344 | −1.451 |
| 17 | NZ | Lys | 312 | 26.764 | 9.682 | −0.652 |
| 18 | HZ1 | Lys | 312 | 27.502 | 9.687 | 0.081 |
| 19 | HZ2 | Lys | 312 | 25.996 | 10.319 | −0.358 |
| 20 | HZ3 | Lys | 312 | 27.171 | 10.035 | −1.541 |
| 21 | C | Lys | 312 | 29.087 | 3.446 | −3.083 |
| 22 | O | Lys | 312 | 28.655 | 2.611 | −2.287 |
| 23 | N | Ser | 313 | 30.038 | 3.175 | −3.970 |
| 24 | HN | Ser | 313 | 30.342 | 3.883 | −4.576 |
| 25 | Cα | Ser | 313 | 30.641 | 1.850 | −4.062 |
| 26 | Hα | Ser | 313 | 29.906 | 1.128 | −3.739 |
| 27 | Cβ | Ser | 313 | 31.036 | 1.546 | −5.508 |
| 28 | Hβ1 | Ser | 313 | 31.618 | 0.636 | −5.536 |
| 29 | Hβ2 | Ser | 313 | 31.625 | 2.362 | −5.898 |
| 30 | Oγ | Ser | 313 | 29.890 | 1.379 | −6.326 |
| 31 | Hγ | Ser | 313 | 29.498 | 0.519 | −6.156 |
| 32 | C | Ser | 313 | 31.86 | 31.749 | −3.157 |
| 33 | O | Ser | 313 | 32.993 | 1.960 | −3.597 |
| 34 | N | Ile | 314 | 31.629 | 1.426 | −1.889 |
| 35 | HN | Ile | 314 | 30.707 | 1.271 | −1.597 |
| 36 | Cα | Ile | 314 | 32.714 | 1.299 | −0.922 |
| 37 | Hα | Ile | 314 | 33.541 | 1.902 | −1.268 |
| 38 | Cβ | Ile | 314 | 32.302 | 1.800 | 0.480 |
| 39 | Hβ | Ile | 314 | 31.913 | 0.958 | 1.034 |
| 40 | Cγ1 | Ile | 314 | 31.217 | 2.876 | 0.382 |
| 41 | Hγ11 | Ile | 314 | 31.001 | 3.250 | 1.372 |
| 42 | Hγ12 | Ile | 314 | 30.323 | 2.440 | −0.036 |
| 43 | Cγ2 | Ile | 314 | 33.514 | 2.334 | 1.231 |
| 44 | Hγ21 | Ile | 314 | 33.648 | 1.771 | 2.144 |
| 45 | Hγ22 | Ile | 314 | 33.362 | 3.376 | 1.471 |
| 46 | Hγ23 | Ile | 314 | 34.395 | 2.232 | 0.615 |
| 47 | Cδ1 | Ile | 314 | 31.608 | 4.054 | −0.485 |
| 48 | Hδ11 | Ile | 314 | 31.114 | 4.944 | −0.127 |
| 49 | Hδ12 | Ile | 314 | 31.312 | 3.863 | −1.506 |
| 501 | Hδ13 | Ile | 314 | 32.678 | 4.194 | −0.441 |
| 51 | C | Ile | 314 | 33.174 | −0.148 | −0.800 |
| 52 | O | Ile | 314 | 32.442 | −1.076 | −1.144 |
| 53 | N | Arg | 315 | 34.388 | −0.330 | −0.296 |
| 54 | HN | Arg | 315 | 34.918 | 0.450 | −0.033 |
| 55 | Cα | Arg | 315 | 34.948 | −1.661 | −0.111 |
| 56 | Hα | Arg | 315 | 34.134 | −2.370 | −0.133 |
| 57 | Cβ | Arg | 315 | 35.932 | −1.991 | −1.235 |
| 58 | Hβ1 | Arg | 315 | 36.239 | −3.022 | −1.136 |
| 59 | Hβ2 | Arg | 315 | 36.799 | −1.356 | −1.133 |
| 60 | Cγ | Arg | 315 | 35.356 | −1.794 | −2.629 |
| 61 | Hγ1 | Arg | 315 | 35.916 | −1.019 | −3.132 |
| 62 | Hγ2 | Arg | 315 | 34.323 | −1.495 | −2.542 |
| 63 | Cδ | Arg | 315 | 35.431 | −3.070 | −3.452 |
| 64 | Hδ1 | Arg | 315 | 36.169 | −3.725 | −3.012 |
| 65 | Hδ2 | Arg | 315 | 35.730 | −2.818 | −4.459 |
| 66 | Nε | Arg | 315 | 34.148 | −3.767 | −3.500 |
| 67 | Hε | Arg | 315 | 33.453 | −3.479 | −2.873 |
| 68 | CZ | Arg | 315 | 33.875 | −4.761 | −4.343 |
| 69 | NH1 | Arg | 315 | 34.792 | −5.178 | −5.207 |
| 70 | HH11 | Arg | 315 | 35.693 | −4.746 | −5.229 |
| 71 | HH12 | Arg | 315 | 34.580 | −5.925 | −5.837 |
| 72 | NH2 | Arg | 315 | 32.682 | −5.340 | −4.321 |
| 73 | HH21 | Arg | 315 | 31.988 | −5.029 | −3.671 |
| 74 | HH22 | Arg | 315 | 32.477 | −6.086 | −4.954 |
| 75 | C | Arg | 315 | 35.648 | −1.756 | 1.239 |
| 76 | O | Arg | 315 | 36.877 | −1.743 | 1.317 |
| 77 | N | Ile | 316 | 34.856 | −1.842 | 2.300 |
| 78 | HN | Ile | 316 | 33.885 | −1.843 | 2.171 |
| 79 | Cα | lIe | 316 | 35.395 | −1.929 | 3.652 |
| 80 | Hα | Ile | 316 | 36.450 | −1.719 | 3.600 |
| 81 | Cβ | Ile | 316 | 34.752 | −0.889 | 4.590 |
| 82 | Hβ | lIe | 316 | 34.830 | −1.256 | 5.603 |
| 83 | Cγ1 | lIe | 316 | 33.277 | −0.685 | 4.244 |
| 84 | Hγ11 | Ile | 316 | 32.752 | −1.621 | 4.360 |
| 85 | Hγ12 | Ile | 316 | 33.195 | −0.353 | 3.219 |
| 86 | Cγ2 | Ile | 316 | 35.506 | 0.430 | 4.508 |
| 87 | Hγ21 | Ile | 316 | 36.499 | 0.255 | 4.122 |
| 88 | Hγ22 | Ile | 316 | 35.574 | 0.867 | 5.493 |
| 89 | Hγ23 | Ile | 316 | 34.978 | 1.106 | −3.851 |
| 90 | Cδ1 | Ile | 316 | 32.600 | 0.342 | 5.119 |
| 91 | Hδ11 | Ile | 316 | 32.099 | 1.069 | 4.498 |
| 92 | Hδ12 | lIe | 316 | 33.343 | 0.837 | 5.726 |
| 93 | Hδ13 | Ile | 316 | 31.879 | −0.146 | 5.756 |
| 94 | C | Ile | 316 | 35.200 | −3.322 | 4.240 |
| 95 | O | Ile | 316 | 34.071 | −3.782 | 4.415 |
| 96 | N | Gln | 317 | 36.309 | −3.987 | 4.543 |
| 97 | HN | Gln | 317 | 37.179 | −3.565 | 4.380 |
| 98 | Cα | Gln | 317 | 36.268 | −5.329 | 5.112 |
| 99 | Hα | Gln | 317 | 35.362 | −5.806 | 4.770 |
| 100 | Cβ | Gln | 317 | 37.473 | −6.142 | 4.630 |
| 101 | Hβ1 | Gln | 317 | 37.941 | −6.617 | 5.479 |
| 102 | Hβ2 | Gln | 317 | 38.183 | −5.470 | 4.170 |
| 103 | Cγ | Gln | 317 | 37.113 | −7.220 | 3.620 |
| 104 | Hγ1 | Gln | 317 | 36.041 | −7.227 | 3.487 |
| 105 | Hγ2 | Gln | 317 | 37.433 | −8.177 | 4.004 |
| 106 | Cδ | Gln | 317 | 37.768 | −6.998 | 2.270 |
| 107 | Oε1 | Gln | 317 | 38.241 | −7.941 | 1.634 |
| 108 | Nε2 | Gln | 317 | 37.798 | −5.748 | 1.825 |
| 109 | Hε21 | Gln | 317 | 37.402 | −5.047 | 2.384 |
| 110 | Hε22 | Gln | 317 | 38.216 | −5.576 | 0.955 |
| 111 | C | Gln | 317 | 36.246 | −5.268 | 6.638 |
| 112 | O | Gln | 317 | 36.026 | −4.207 | 7.221 |
| 113 | N | Arg | 318 | 36.473 | −6.413 | 7.280 |
| 114 | HN | Arg | 318 | 36.639 | −7.226 | 6.759 |
| 115 | Cα | Arg | 318 | 36.477 | −6.491 | 8.739 |
| 116 | Hα | Arg | 318 | 35.456 | −6.407 | 9.077 |
| 117 | Cβ | Arg | 318 | 37.040 | −7.838 | 9.195 |
| 118 | Hβ1 | Arg | 318 | 37.263 | −7.783 | 10.251 |
| 119 | Hβ2 | Arg | 318 | 37.954 | −8.035 | 8.654 |
| 120 | Cγ | Arg | 318 | 36.090 | −9.003 | 8.969 |
| 121 | Hγ1 | Arg | 318 | 35.096 | −8.616 | 8.796 |
| 122 | Hγ2 | Arg | 318 | 36.086 | −9.628 | 9.849 |
| 123 | Cδ | Arg | 318 | 36.509 | −9.842 | 7.772 |
| 124 | Hδ1 | Arg | 318 | 37.283 | −10.528 | 8.082 |
| 125 | Hδ2 | Arg | 318 | 36.896 | −9.186 | 7.006 |
| 126 | Nε | Arg | 318 | 35.392 | −10.605 | 7.221 |
| 127 | Hε | Arg | 318 | 34.960 | −10.257 | 6.413 |
| 128 | CZ | Arg | 318 | 34.934 | −11.736 | 7.752 |
| 129 | NH1 | Arg | 318 | 35.494 | −12.239 | 8.845 |
| 130 | HH11 | Arg | 318 | 36.264 | −11.769 | 9.275 |
| 131 | HH12 | Arg | 318 | 35.145 | −13.090 | 9.239 |
| 132 | NH2 | Arg | 318 | 33.912 | −12.367 | 7.188 |
| 133 | HH21 | Arg | 318 | 33.487 | −11.992 | 6.364 |
| 134 | HH22 | Arg | 318 | 33.568 | −13.217 | 7.587 |
| 135 | C | Arg | 318 | 37.292 | −5.354 | 9.351 |
| 136 | O | Arg | 318 | 36.860 | −4.714 | 10.310 |
| 137 | N | Gly | 319 | 38.470 | −5.105 | 8.789 |
| 138 | HN | Gly | 319 | 33.763 | −5.645 | 8.025 |
| 139 | Cα | Gly | 319 | 39.322 | −4.043 | 9.291 |
| 140 | Hα1 | Gly | 319 | 38.933 | −3.703 | 10.240 |
| 141 | Hα2 | Gly | 319 | 39.301 | −3.220 | 8.592 |
| 142 | C | Gly | 319 | 40.760 | −4.487 | 9.482 |
| 143 | O | Gly | 319 | 41.566 | −4.397 | 8.555 |
| 144 | N | Pro | 320 | 41.116 | −4.973 | 10.683 |
| 145 | Cα | Pro | 320 | 42.478 | −5.431 | 10.978 |
| 146 | Hα | Pro | 320 | 43.198 | −4.637 | 10.843 |
| 147 | Cβ | Pro | 320 | 42.418 | −5.824 | 12.459 |
| 148 | Hβ1 | Pro | 320 | 42.808 | −5.019 | 13.063 |
| 149 | Hβ2 | Pro | 320 | 43.003 | −6.718 | 12.619 |
| 150 | Cγ | Pro | 320 | 40.973 | −6.061 | 12.737 |
| 151 | Hγ1 | Pro | 320 | 40.759 | −5.847 | 13.774 |
| 152 | Hγ2 | Pro | 320 | 40.717 | −7.084 | 12.503 |
| 153 | Cδ | Pro | 320 | 40.222 | −5.117 | 11.844 |
| 154 | Hδ1 | Pro | 320 | 40.074 | −4.167 | 12.336 |
| 155 | Hδ2 | Pro | 320 | 39.274 | −5.546 | 11.552 |
| 156 | C | Pro | 320 | 42.883 | −6.627 | 10.124 |
| 157 | O | Pro | 320 | 42.431 | −7.749 | 10.355 |
| 158 | N | Gly | 321 | 43.736 | −6.380 | 9.136 |
| 159 | HN | Gly | 321 | 44.062 | −5.466 | 8.999 |
| 160 | Cα | Gly | 321 | 44.187 | −7.446 | 8.260 |
| 161 | Hα1 | Gly | 321 | 43.719 | −8.370 | 8.564 |
| 162 | Hα2 | Gly | 321 | 45.258 | −7.548 | 8.358 |
| 163 | C | Gly | 321 | 43.851 | −7.182 | 6.805 |
| 164 | O | Gly | 321 | 44.534 | −7.671 | 5.905 |
| 165 | N | Arg | 322 | 42.795 | −6.409 | 6.574 |
| 166 | HN | Arg | 322 | 42.290 | −6.0507 | 7.333 |
| 167 | Cα | Arg | 322 | 42.368 | −6.081 | 5.219 |
| 168 | Hα | Arg | 322 | 42.949 | −6.683 | 4.535 |
| 169 | Cβ | Arg | 322 | 40.884 | −6.413 | 5.036 |
| 170 | Hβ1 | Arg | 322 | 40.342 | −5.499 | 4.844 |
| 171 | Hβ2 | Arg | 322 | 40.513 | −6.856 | 5.948 |
| 172 | Cγ | Arg | 322 | 40.612 | −7.378 | −3.893 |
| 173 | Hγ1 | Arg | 322 | 40.874 | −6.898 | 2.961 |
| 174 | Hγ2 | Arg | 322 | 39.562 | −7.627 | 3.888 |
| 175 | Cδ | Arg | 322 | 41.421 | −8.658 | 4.034 |
| 176 | Hδ1 | Arg | 322 | 41.908 | −8.658 | 4.999 |
| 177 | Hδ2 | Arg | 322 | 42.167 | −8.685 | 3.255 |
| 178 | Nε | Arg | 322 | 40.582 | −9.850 | 3.927 |
| 179 | Hε | Arg | 322 | 39.618 | −9.738 | 4.063 |
| 180 | CZ | Arg | 322 | 41.052 | −11.065 | 3.657 |
| 181 | NH1 | Arg | 322 | 42.352 | −11.255 | 3.467 |
| 182 | HH11 | Arg | 322 | 42.985 | −10.483 | 3.526 |
| 183 | HH12 | Arg | 322 | 42.698 | −12.171 | 3.264 |
| 184 | NH2 | Arg | 322 | 40.220 | −12.095 | 3.577 |
| 185 | HH21 | Arg | 322 | 39.239 | −11.958 | 3.720 |
| 186 | HH22 | Arg | 322 | 40.572 | −13.008 | 3.374 |
| 187 | C | Arg | 322 | 42.619 | −4.608 | 4.911 |
| 188 | O | Arg | 322 | 43.323 | −3.920 | 5.651 |
| 189 | N | Ala | 323 | 42.039 | −4.129 | 3.815 |
| 190 | HN | Ala | 323 | 41.489 | −4.726 | 3.265 |
| 191 | Cα | Ala | 323 | 42.200 | −2.738 | 3.411 |
| 192 | Hα | Ala | 323 | 42.629 | −2.197 | 4.242 |
| 193 | Cβ | Ala | 323 | 43.160 | −2.639 | 2.235 |
| 194 | Hβ1 | Ala | 323 | 43.288 | −1.602 | 1.961 |
| 195 | Hβ2 | Ala | 323 | 42.758 | −3.186 | 1.395 |
| 196 | Hβ3 | Ala | 323 | 44.115 | −3.058 | 2.514 |
| 197 | C | Ala | 323 | 40.856 | −2.111 | 3.054 |
| 198 | O | Ala | 323 | 39.907 | −2.812 | 2.705 |
| 199 | N | Phe | 324 | 40.783 | −0.787 | 3.144 |
| 200 | HN | Phe | 324 | 41.574 | −0.282 | 3.427 |
| 201 | Cα | Phe | 324 | 39.554 | −0.067 | 2.830 |
| 202 | Hα | Phe | 324 | 38.757 | −0.792 | 2.749 |
| 203 | Cβ | Phe | 324 | 39.215 | 0.922 | 3.947 |
| 204 | Hβ1 | Phe | 324 | 38.270 | 1.395 | 3.723 |
| 205 | Hβ2 | Phe | 324 | 39.985 | 1.677 | 3.994 |
| 206 | Cγ | Phe | 324 | 39.106 | 0.291 | 5.307 |
| 207 | Cδ1 | Phe | 324 | 38.525 | −0.958 | 5.465 |
| 208 | Hδ1 | Phe | 324 | 38.150 | −1.482 | 4.598 |
| 209 | Cδ2 | Phe | 324 | 39.582 | 0.950 | 6.429 |
| 210 | Hδ2 | Phe | 324 | 40.037 | 1.923 | 6.318 |
| 211 | Cε1 | Phe | 324 | 38.422 | −1.537 | 6.716 |
| 212 | Hε1 | Phe | 324 | 37.966 | −2.510 | 6.826 |
| 213 | Cε2 | Phe | 324 | 39.482 | 0.376 | 7.682 |
| 214 | Hε2 | Phe | 324 | 39.858 | 0.901 | 8.548 |
| 215 | CZ | Phe | 324 | 38.901 | −0.868 | 7.826 |
| 216 | HZ | Phe | 324 | 38.822 | −1.318 | 8.805 |
| 217 | C | Phe | 324 | 39.680 | 0.676 | 1.504 |
| 218 | O | Phe | 324 | 40.666 | 1.372 | 1.262 |
| 219 | N | Val | 325 | 38.672 | 0.528 | 0.650 |
| 220 | HN | Val | 325 | 37.912 | −0.037 | 0.901 |
| 221 | Cα | Val | 325 | 38.668 | 1.190 | −0.650 |
| 222 | Hα | Val | 325 | 39.403 | 1.982 | −0.623 |
| 223 | Cβ | Val | 325 | 39.041 | 0.222 | −1.790 |
| 224 | Hβ | Val | 325 | 38.151 | −0.311 | −2.085 |
| 225 | Cγ1 | Val | 325 | 39.551 | 0.993 | −2.998 |
| 226 | Hγ11 | Val | 325 | 38.751 | 1.593 | −3.406 |
| 227 | Hγ12 | Val | 325 | 39.899 | 0.299 | −3.748 |
| 228 | Hγ13 | Val | 325 | 40.365 | 1.636 | −2.697 |
| 229 | Cγ2 | Val | 325 | 40.075 | −0.794 | −1.325 |
| 230 | Hγ21 | Val | 325 | 40.694 | −0.352 | −0.557 |
| 231 | Hγ22 | Val | 325 | 40.693 | −1.088 | −2.160 |
| 232 | Hγ23 | Val | 325 | 39.572 | −1.662 | −0.926 |
| 233 | C | Val | 325 | 37.301 | 1.795 | −0.947 |
| 234 | O | Val | 325 | 36.347 | 1.080 | −1.254 |
| 235 | N | Thr | 326 | 37.213 | 3.116 | −0.849 |
| 236 | HN | Thr | 326 | 38.003 | 3.628 | −0.598 |
| 237 | Cα | Thr | 326 | 35.966 | 3.821 | −1.101 |
| 238 | Hα | Thr | 326 | 35.164 | 3.115 | −0.975 |
| 239 | Cβ | Thr | 326 | 35.787 | 4.960 | −0.097 |
| 240 | Hβ | Thr | 326 | 34.770 | 5.322 | −0.156 |
| 241 | Oγ1 | Thr | 326 | 36.659 | 6.035 | −0.398 |
| 242 | Hγ1 | Thr | 326 | 36.475 | 6.772 | 0.190 |
| 243 | Cγ2 | Thr | 326 | 36.048 | 4.544 | 1.334 |
| 244 | Hγ21 | Thr | 326 | 35.764 | 3.510 | 1.466 |
| 245 | Hγ22 | Thr | 326 | 35.469 | 5.164 | 2.002 |
| 246 | Hγ23 | Thr | 326 | 37.099 | 4.659 | 1.556 |
| 247 | C | Thr | 326 | 35.921 | 4.368 | −2.523 |
| 248 | O | Thr | 326 | 36.757 | 5.184 | −2.912 |
| 249 | N | Ile | 327 | 34.939 | 3.914 | −3.296 |
| 250 | HN | Ile | 327 | 34.303 | 3.266 | −2.929 |
| 251 | Cα | Ile | 327 | 34.784 | 4.360 | −4.675 |
| 252 | Hα | Ile | 327 | 35.656 | 4.942 | −4.938 |
| 253 | Cβ | Ile | 327 | 34.685 | 3.171 | −5.650 |
| 254 | Hβ | Ile | 327 | 33.692 | 2.755 | −5.573 |
| 255 | Cγ1 | Ile | 327 | 35.709 | 2.093 | −5.290 |
| 256 | Hγ11 | Ile | 327 | 35.635 | 1.873 | −4.234 |
| 257 | Hγ12 | Ile | 327 | 36.700 | 2.459 | −5.508 |
| 258 | Cγ2 | Ile | 327 | 34.888 | 3.643 | −7.082 |
| 259 | Hγ21 | Ile | 327 | 34.113 | 4.348 | −7.342 |
| 260 | Hγ22 | Ile | 327 | 34.844 | 2.795 | −7.750 |
| 261 | Hγ23 | Ile | 327 | 35.853 | 4.120 | −7.170 |
| 262 | Cδ1 | Ile | 327 | 35.515 | 0.799 | −6.050 |
| 263 | Hδ11 | Ile | 327 | 35.213 | 1.019 | −7.063 |
| 264 | Hδ12 | Ile | 327 | 34.751 | 0.208 | −5.566 |
| 265 | Hδ13 | Ile | 327 | 36.443 | 0.247 | −6.063 |
| 266 | C | Ile | 327 | 33.544 | 5.232 | −4.832 |
| 267 | O | Ile | 327 | 32.442 | 4.838 | −4.448 |
| 268 | N | Gly | 328 | 33.730 | 6.418 | −5.400 |
| 269 | HN | Gly | 328 | 34.630 | 6.677 | −5.687 |
| 270 | Cα | Gly | 328 | 32.618 | 7.328 | −5.599 |
| 271 | Hα1 | Gly | 328 | 32.616 | 8.057 | −4.802 |
| 272 | Hα2 | Gly | 328 | 31.696 | 6.767 | −5.560 |
| 273 | C | Gly | 328 | 32.693 | 8.055 | −6.927 |
| 274 | O | Gly | 328 | 33.306 | 7.565 | −7.876 |

Column Headings:
A = atom serial no. in peptide
B = atom type/position in amino acid
C = name of amino acid residue
D = residue number in peptide sequence
X, Y, Z = Orthogonal Coordinates (in Angstroms) for X, Y and Z axes

TABLE 5
Average NMR Coordinates of Internally Constrained X4
Mimic Peptide 1 (“X4-M1)”

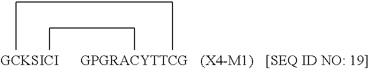

| A | B | C | D | X | Y | Z |
|---|---|---|---|---|---|---|
| 1 | N | Gly | 310 | −10.047 | 0.815 | 5.820 |
| 2 | H | Gly | 310 | −9.654 | −0.037 | 5.473 |
| 3 | Cα | Gly | 310 | −9.255 | 2.006 | 5.562 |
| 4 | 1Hα | Gly | 310 | −9.900 | 2.884 | 5.567 |
| 5 | 2Hα | Gly | 310 | −8.526 | 2.142 | 6.362 |
| 6 | Qα | Gly | 310 | −9.213 | 2.513 | 5.964 |
| 7 | C | Gly | 310 | −8.532 | 1.904 | 4.218 |
| 8 | O | Gly | 310 | −7.354 | 1.553 | 4.167 |
| 9 | N | Cys | 311 | −9.268 | 2.216 | 3.161 |
| 10 | H | Cys | 311 | −10.226 | 2.500 | 3.211 |
| 11 | Cα | Cys | 311 | −8.712 | 2.164 | 1.820 |
| 12 | Hα | Cys | 311 | −8.267 | 1.176 | 1.704 |
| 13 | Cβ | Cys | 311 | −9.799 | 2.303 | 0.752 |
| 14 | 1Hβ | Cys | 311 | −9.957 | 3.362 | 0.550 |
| 15 | 2Hβ | Cys | 311 | −9.438 | 1.853 | −0.174 |
| 16 | Qβ | Cys | 311 | −9.698 | 2.607 | 0.188 |
| 17 | Sγ | Cys | 311 | −11.405 | 1.542 | 1.187 |
| 18 | C | Cys | 311 | −7.649 | 3.257 | 1.698 |
| 19 | O | Cys | 311 | −7.905 | 4.317 | 1.128 |
| 20 | N | Lys | 312 | −6.477 | 2.962 | 2.242 |
| 21 | H | Lys | 312 | −6.277 | 2.098 | 2.704 |
| 22 | Cα | Lys | 312 | −5.374 | 3.907 | 2.201 |
| 23 | Hα | Lys | 312 | −5.707 | 4.773 | 1.630 |
| 24 | Cβ | Lys | 312 | −5.036 | 4.397 | 3.611 |
| 25 | 1Hβ | Lys | 312 | −4.445 | 3.643 | 4.130 |
| 26 | 2Hβ | Lys | 312 | −4.423 | 5.296 | 3.550 |
| 27 | Qβ | Lys | 312 | −4.434 | 4.470 | 3.840 |
| 28 | Cγ | Lys | 312 | −6.308 | 4.694 | 4.408 |
| 29 | 1Hγ | Lys | 312 | −6.942 | 3.808 | 4.431 |
| 30 | 2Hγ | Lys | 312 | −6.048 | 4.928 | 5.440 |
| 31 | Qγ | Lys | 312 | −6.495 | 4.368 | 4.936 |
| 32 | Cδ | Lys | 312 | −7.080 | 5.864 | 3.793 |
| 33 | 1Hδ | Lys | 312 | −6.871 | 6.776 | 4.353 |
| 34 | 2Hδ | Lys: | 312 | −6.739 | 6.032 | 2.772 |
| 35 | Qδ | Lys | 312 | −6.805 | 6.404 | 3.562 |
| 36 | Cε | Lys | 312 | −8.585 | 5.590 | 3.797 |
| 37 | 1Hε | Lys | 312 | −9.126 | 6.495 | 3.521 |
| 38 | 2Hε | Lys | 312 | −8.825 | 4.834 | 3.049 |
| 39 | Qε | Lys | 312 | −8.975 | 5.664 | 3.285 |
| 40 | NZ | Lys | 312 | −9.025 | 5.130 | 5.133 |
| 41 | 1HZ | Lys | 312 | −8.996 | 5.876 | 5.820 |
| 42 | 2HZ | Lys | 312 | −9.976 | 4.779 | 5.120 |
| 43 | QZ | Lys | 312 | −9.486 | 5.327 | 5.470 |
| 44 | C | Lys | 312 | −4.190 | 3.270 | 1.471 |
| 45 | O | Lys | 312 | −4.240 | 2.097 | 1.106 |
| 46 | N | Ser | 313 | −3.153 | 4.072 | 1.281 |
| 47 | H | Ser | 313 | −3.120 | 5.026 | 1.581 |
| 48 | Cα | Ser | 313 | −1.958 | 3.602 | 0.601 |
| 49 | Hα | 5cr | 313 | −2.298 | 3.258 | −0.376 |
| 50 | Cβ | Ser | 313 | −0.946 | 4.735 | 0.418 |
| 51 | 1Hβ | Ser | 313 | −1.251 | 5.593 | 1.017 |
| 52 | 2Hβ | Ser | 313 | 0.027 | 4.415 | 0.790 |
| 53 | Qβ | Ser | 313 | −0.612 | 5.004 | 0.904 |
| 54 | Oγ | Ser | 313 | −0.826 | 5.128 | −0.947 |
| 55 | Hγ | Set | 313 | 0.079 | 5.520 | −1.111 |
| 56 | C | Ser | 313 | −1.328 | 2.450 | 1.386 |
| 57 | O | Ser | 313 | −1.066 | 2.578 | 2.581 |
| 58 | N | Ile | 314 | −1.104 | 1.349 | 0.683 |
| 59 | H | Ile | 314 | −1.321 | 1.252 | −0.288 |
| 60 | Cα | Ile | 314 | −0.511 | 0.175 | 1.300 |
| 61 | Hα | Ile | 314 | −0.280 | 0.430 | 2.334 |
| 62 | Cβ | Ile | 314 | −1.515 | −0.979 | 1.332 |
| 63 | Hβ | Ile | 314 | −2.479 | −0.584 | 1.652 |
| 64 | Qγ2 | Ile | 314 | −1.757 | −1.716 | −0.396 |
| 65 | Cγ2 | Ile | 314 | −1.711 | −1.574 | −0.064 |
| 66 | 1Hγ2 | Ile | 314 | −1.156 | −2.509 | −0.142 |
| 67 | 2Hγ2 | Ile | 314 | −2.771 | −1.765 | −0.232 |
| 68 | 3Hγ2 | Ile | 314 | −1.345 | −0.872 | −0.813 |
| 69 | Cγ1 | Ile | 314 | −1.099 | −2.039 | 2.354 |
| 70 | 1Hγ1 | Ile | 314 | −1.140 | −1.617 | 3.358 |
| 71 | 2Hγ1 | Ile | 314 | −1.804 | −2.870 | 2.328 |
| 72 | Qγ1 | Ile | 314 | −1.472 | −2.244 | 2.843 |
| 73 | Qδ1 | Ile | 314 | 0.649 | −2.675 | 1.999 |
| 74 | Cδ1 | Ile | 314 | 0.313 | −2.553 | 2.067 |
| 75 | 1Hδ1 | Ile | 314 | 0.464 | −2.619 | 0.990 |
| 76 | 2Hδ1 | Ile | 314 | 1.044 | −1.866 | 2.495 |
| 77 | 3Hδ1 | Ile | 314 | 0.438 | −3.540 | 2.513 |
| 78 | C | Ile | 314 | 0.796 | −0.167 | 0.582 |
| 79 | O | Ile | 314 | 0.799 | −0.940 | −0.375 |
| 80 | N | Cys | 315 | 1.876 | 0.425 | 1.071 |
| 81 | H | Cys | 315 | 1.865 | 1.052 | 1.850 |
| 82 | Cα | Cys | 315 | 3.186 | 0.193 | 0.489 |
| 83 | Hα | Cys | 315 | 3.025 | −0.026 | −0.567 |
| 84 | Cβ | Cys | 315 | 4.069 | 1.441 | 0.566 |
| 85 | 1Hβ | Cys | 315 | 3.430 | 2.312 | 0.708 |
| 86 | 2Hβ | Cys | 315 | 4.705 | 1.364 | 1.448 |
| 87 | Qβ | Cys | 315 | 4.067 | 1.838 | 1.078 |
| 88 | Sγ | Cys | 315 | 5.129 | 1.723 | −0.899 |
| 89 | C | Cys | 315 | 3.824 | −1.002 | 1.200 |
| 90 | O | Cys | 315 | 3.879 | −1.040 | 2.428 |
| 91 | N | Ile | 316 | 4.289 | −1.948 | 0.398 |
| 92 | H | Ile | 316 | 4.240 | −1.909 | −0.600 |
| 93 | Cα | Ile | 316 | 4.921 | −3.141 | 0.936 |
| 94 | Hα | Ile | 316 | 4.470 | −3.339 | 1.908 |
| 95 | Cβ | Ile | 316 | 4.626 | −4.351 | 0.046 |
| 96 | Hβ | Ile | 316 | 4.892 | −4.094 | −0.979 |
| 97 | Qγ2 | Ile | 316 | 5.690 | −5.835 | 0.545 |
| 98 | Cγ2 | Ile | 316 | 5.486 | −5.550 | 0.449 |
| 99 | 1Hγ2 | Ile | 316 | 5.717 | −5.491 | 1.512 |
| 100 | 2Hγ2 | Ile | 316 | 4.941 | −6.472 | 0.247 |
| 101 | 3Hγ2 | Ile | 316 | 6.412 | −5.542 | −0.125 |
| 102 | Cγ1 | Ile | 316 | 3.133 | −4.687 | 0.055 |
| 103 | 1Hγ1 | Ile | 316 | 2.996 | −5.754 | −0.117 |
| 104 | 2Hγ1 | Ile | 316 | 2.713 | −4.465 | 1.036 |
| 105 | Qγ1 | Ile | 316 | 2.854 | −5.110 | 0.460 |
| 106 | Qδ1 | Ile | 316 | 2.208 | −3.703 | −1.272 |
| 107 | Cδ1 | Ile | 316 | 2.386 | −3.892 | −1.017 |
| 108 | 1Hδ1 | Ile | 316 | 1.558 | −3.351 | −0.558 |
| 109 | 2Hδ1 | Ile | 316 | 3.068 | −3.183 | −1.486 |
| 110 | 3Hδ1 | Ile | 316 | 1.998 | −4.576 | −1.772 |
| 111 | C | Ile | 316 | 6.414 | −2.876 | 1.137 |
| 112 | O | Ile | 316 | 7.137 | −3.733 | 1.643 |
| 113 | N | Gly | 319 | 6.831 | −1.685 | 0.732 |
| 114 | H | Gly | 319 | 6.236 | −0.994 | 0.322 |
| 115 | Cα | Gly | 319 | 8.225 | −1.297 | 0.862 |
| 116 | 1Hα | Gly | 319 | 8.663 | −1.785 | 1.732 |
| 117 | 2Hα | Gly | 319 | 8.784 | −1.637 | −0.010 |
| 118 | Qα | Gly | 319 | 8.723 | −1.711 | 0.861 |
| 119 | C | Gly | 319 | 8.360 | 0.221 | 1.001 |
| 120 | O | Gly | 319 | 7.402 | 0.957 | 0.768 |
| 121 | N | Pro | 320 | 9.589 | 0.656 | 1.389 |
| 122 | Cδ | Pro | 320 | 10.747 | −0.187 | 1.673 |
| 123 | Cα | Pro | 320 | 9.862 | 2.072 | 1.561 |
| 124 | Hα | Pro | 320 | 9.095 | 2.520 | 2.021 |
| 125 | Cβ | Pro | 320 | 11.131 | 2.130 | 2.396 |
| 126 | 1Hβ | Pro | 320 | 11.800 | 2.912 | 2.036 |
| 127 | 2Hβ | Pro | 320 | 10.904 | 2.358 | 3.437 |
| 128 | Qβ | Pro | 320 | 11.352 | 2.635 | 2.737 |
| 129 | Cγ | Pro | 320 | 11.777 | 0.760 | 2.267 |
| 130 | 1Hγ | Pro | 320 | 12.660 | 0.812 | 1.630 |
| 131 | 2Hγ | Pro | 320 | 12.108 | 0.401 | 3.242 |
| 132 | Qγ | Pro | 320 | 12.384 | 0.606 | 2.436 |
| 133 | 1Hδ | Pro | 320 | 11.122 | −0.663 | 0.767 |
| 134 | 2Hδ | Pro | 320 | 10.494 | −0.986 | 2.370 |
| 135 | Qδ | Pro | 320 | 10.808 | −0.824 | 1.568 |
| 136 | C | Pro | 320 | 10.004 | 2.772 | 0.208 |
| 137 | O | Pro | 320 | 10.987 | 3.471 | −0.033 |
| 138 | N | Gly | 321 | 9.008 | 2.560 | −0.639 |
| 139 | H | Gly | 321 | 8.212 | 1.989 | −0.435 |
| 140 | Cα | Gly | 321 | 9.009 | 3.161 | −1.962 |
| 141 | 1Hα | Gly | 321 | 8.218 | 3.908 | −2.027 |
| 142 | 2Hα | Gly | 321 | 9.953 | 3.682 | −2.127 |
| 143 | Qα | Gly | 321 | 9.085 | 3.795 | −2.077 |
| 144 | C | Gly | 321 | 8.808 | 2.101 | −3.047 |
| 145 | O | Gly | 321 | 9.456 | 2.146 | −4.091 |
| 146 | N | Arg | 322 | 7.908 | 1.172 | −2.762 |
| 147 | H | Arg | 322 | 7.385 | 1.143 | −1.910 |
| 148 | Cα | Arg | 322 | 7.613 | 0.103 | −3.700 |
| 149 | Hα | Arg | 322 | 8.323 | 0.243 | −4.515 |
| 150 | Cβ | Arg | 322 | 7.813 | −1.269 | −3.052 |
| 151 | 1Hβ | Arg | 322 | 8.527 | −1.187 | −2.233 |
| 152 | 2Hβ | Arg | 322 | 6.872 | −1.613 | −2.622 |
| 153 | Qβ | Arg | 322 | 7.700 | −1.400 | −2.427 |
| 154 | Cγ | Arg | 322 | 8.316 | −2.290 | −4.075 |
| 155 | 1Hγ | Arg | 322 | 7.805 | −3.241 | −3.926 |
| 156 | 2Hγ | Arg | 322 | 8.072 | −1.952 | −−5.082 |
| 157 | Qγ | Arg | 322 | 7.938 | −2.597 | −4.504 |
| 158 | Cδ | Arg | 322 | 9.827 | −2.492 | −3.950 |
| 159 | 1Hδ | Arg | 322 | 10.351 | −1.797 | −4.607 |
| 160 | 2Hδ | Arg | 322 | 10.149 | −2.273 | −2.932 |
| 161 | Qδ | Arg | 322 | 10.250 | −2.035 | −3.769 |
| 162 | Nε | Arg | 322 | 10.182 | −3.885 | −4.304 |
| 163 | Hε | Arg | 322 | 9.487 | −4.447 | −4.752 |
| 164 | CZ | Arg | 322 | 11.387 | −4.444 | −4.060 |
| 165 | NH1 | Arg | 322 | 11.595 | −5.697 | −4.419 |
| 166 | NH2 | Arg | 322 | 12.363 | −3.732 | −3.458 |
| 167 | 1HH2 | Arg | 322 | 13.253 | −4.154 | −3.281 |
| 168 | 2HH2 | Arg | 322 | 12.197 | −2.783 | −3.189 |
| 169 | QH2 | Arg | 322 | 12.725 | −3.468 | −3.235 |
| 170 | C | Arg | 322 | 6.172 | 0.217 | −4.200 |
| 171 | O | Arg | 322 | 5.636 | 1.319 | −4.313 |
| 172 | 1HH | Arg | 322 | 12.456 | −6.184 | −4.274 |
| 173 | N | Ala | 323 | 5.585 | −0.936 | −4.487 |
| 174 | H | Ala | 323 | 6.028 | −1.827 | −4.392 |
| 175 | Cα | Ala | 323 | 4.216 | −0.978 | −4.973 |
| 176 | Hα | Ala | 323 | 4.106 | −0.197 | −5.724 |
| 177 | Qβ | Ala | 323 | 3.886 | −2.657 | −5.784 |
| 178 | Cβ | Ala | 323 | 3.949 | −2.335 | −5.629 |
| 179 | 1Hβ | Ala | 323 | 4.372 | −2.342 | −6.633 |
| 180 | 2Hβ | Ala | 323 | 4.411 | −3.123 | −5.034 |
| 181 | 3Hβ | Ala | 323 | 2.874 | −2.505 | −5.686 |
| 182 | C | Ala | 323 | 3.259 | −0.693 | −3.813 |
| 183 | O | Ala | 323 | 3.056 | −1.543 | −2.948 |
| 184 | N | Cys | 324 | 2.696 | 0.506 | −3.834 |
| 185 | H | Cys | 324 | 2.867 | 1.192 | −4.542 |
| 186 | Cα | Cys | 324 | 1.766 | 0.914 | −2.795 |
| 187 | Hα | Cys | 324 | 1.653 | 0.063 | −2.124 |
| 188 | Cβ | Cys | 324 | 2.316 | 2.078 | −1.968 |
| 189 | 1Hβ | Cys | 324 | 1.634 | 2.923 | −2.058 |
| 190 | 2Hβ | Cys | 324 | 2.325 | 1.786 | −0.918 |
| 191 | Qβ | Cys | 324 | 1.980 | 2.354 | −1.488 |
| 192 | Sγ | Cys | 324 | 3.996 | 2.627 | −2.439 |
| 193 | C | Cys | 324 | 0.431 | 1.265 | −3.457 |
| 194 | O | Cys | 324 | 0.363 | 2.178 | −4.279 |
| 195 | N | Tyr | 325 | −0.596 | 0.521 | −3.074 |
| 196 | H | Tyr | 325 | −0.532 | −0.220 | −2.405 |
| 197 | Cα | Tyr | 325 | −1.924 | 0.742 | −3.620 |
| 198 | Hα | Tyr | 325 | −1.872 | 1.594 | −4.297 |
| 199 | Cβ | Tyr | 325 | −2.322 | −0.567 | −4.304 |
| 200 | 1Hβ | Tyr | 325 | −3.351 | −0.484 | −4.655 |
| 201 | 2Hβ | Tyr | 325 | −1.696 | −0.710 | −5.184 |
| 202 | Qβ | Tyr | 325 | −2.524 | −0.597 | −4.920 |
| 203 | Qδ | Tyr | 325 | −2.193 | −1.917 | −3.320 |
| 204 | Qε | Tyr | 325 | −1.996 | −3.965 | −1.827 |
| 205 | QR | Tyr | 325 | −2.094 | −2.941 | −2.574 |
| 206 | Cγ | Tyr | 325 | −2.204 | −1.800 | −3.405 |
| 207 | Cδ1 | Tyr | 325 | −0.961 | −2.321 | −3.109 |
| 208 | 1Hδ | Tyr | 325 | −0.063 | −1.854 | −3.516 |
| 209 | Cε1 | Tyr | 325 | −0.849 | −3.481 | −2.263 |
| 210 | 1Hε | Tyr | 325 | 0.126 | −3.903 | −2.022 |
| 211 | CZ | Tyr | 325 | −1.989 | −4.039 | −1.773 |
| 212 | Cε2 | Tyr | 325 | −3.229 | −3.551 | −2.045 |
| 213 | 2Hε | Tyr | 325 | −4.118 | −4.027 | −1.632 |
| 214 | Cδ2 | Tyr | 325 | −3.340 | −2.390 | −2.891 |
| 215 | 2Hδ | Tyr | 325 | −4.322 | −1.979 | −3.125 |
| 216 | OH | Tyr | 325 | −1.883 | −5.134 | −0.974 |
| 217 | HH | Tyr | 325 | −1.783 | −5.957 | −1.534 |
| 218 | C | Tyr | 325 | −2.930 | 1.049 | −2.508 |
| 219 | O | Tyr | 325 | −2.655 | 0.806 | −1.334 |
| 220 | N | Thr | 326 | −4.073 | 1.578 | −2.918 |
| 221 | H | Thr | 326 | −4.289 | 1.772 | −3.875 |
| 222 | Cα | Thr | 326 | −5.121 | 1.921 | −1.971 |
| 223 | Hα | Thr | 326 | −4.652 | 2.258 | −1.046 |
| 224 | Cβ | Thr | 326 | −5.946 | 3.061 | −2.571 |
| 225 | Hβ | Thr | 326 | −5.888 | 3.053 | −3.659 |
| 226 | Qγ2 | Thr | 326 | −7.748 | 3.036 | −1.989 |
| 227 | Oγ1 | Thr | 326 | −5.404 | 4.234 | −1.971 |
| 228 | 1Hγ | Thr | 326 | −5.722 | 5.046 | −2.460 |
| 229 | Cγ2 | Thr | 326 | −7.402 | 3.040 | −2.101 |
| 230 | 1Hγ2 | Thr | 326 | −7.432 | 2.955 | −1.015 |
| 231 | 2Hγ2 | Thr | 326 | −7.895 | 3.963 | −2.406 |
| 232 | 3Hγ2 | Thr | 326 | −7.916 | 2.189 | −2.547 |
| 233 | C | Thr | 326 | −5.954 | 0.684 | −1.627 |
| 234 | O | Thr | 326 | −6.829 | 0.287 | −2.395 |
| 235 | N | Thr | 327 | −5.653 | 0.110 | −0.471 |
| 236 | H | Thr | 327 | −4.940 | 0.440 | 0.148 |
| 237 | Cα | Thr | 327 | −6.362 | −1.073 | −0.016 |
| 238 | Hα | Thr | 327 | −7.148 | −1.301 | −0.736 |
| 239 | Cβ | Thr | 327 | −5.365 | −2.232 | 0.036 |
| 240 | Hβ | Thr | 327 | −4.689 | −2.203 | −0.818 |
| 241 | Qγ2 | Thr | 327 | −4.397 | −2.283 | 1.663 |
| 242 | Oγ1 | Thr | 327 | −6.186 | −3.395 | 0.085 |
| 243 | 1Hγ | Thr | 327 | −6.205 | −3.839 | −0.810 |
| 244 | Cγ2 | Thr | 327 | −4.582 | −2.273 | 1.350 |
| 245 | 1Hγ2 | Thr | 327 | −3.654 | −2.825 | 1.203 |
| 246 | 2Hγ2 | Thr | 327 | −4.354 | −1.256 | 1.669 |
| 247 | 3Hγ2 | Thr | 327 | −5.182 | −2.768 | 2.115 |
| 248 | C | Thr | 327 | −7.048 | −0.801 | 1.325 |
| 249 | O | Thr | 327 | −6.478 | −0.145 | 2.196 |
| 250 | N | Cys | 328 | −8.261 | −1.319 | 1.449 |
| 251 | H | Cys | 328 | −8.717 | −1.851 | 0.736 |
| 252 | Cα | Cys | 328 | −9.030 | −1.140 | 2.669 |
| 253 | Hα | Cys | 328 | −8.815 | −0.134 | 3.029 |
| 254 | Cβ | Cys | 328 | −10.535 | −1.218 | 2.405 |
| 255 | 1Hβ | Cys | 328 | −10.693 | −1.544 | 1.377 |
| 256 | 2Hβ | Cys | 328 | −10.964 | −1.983 | 3.052 |
| 257 | Qβ | Cys | 328 | −10.828 | −1.764 | 2.214 |
| 258 | Sγ | Cys | 328 | −11.443 | 0.349 | 2.669 |
| 259 | C | Cys | 328 | −8.573 | −2.190 | 3.683 |
| 260 | O | Cys | 328 | −9.136 | −2.292 | 4.772 |
| 261 | N | Gly | 328 | −7.556 | −2.943 | 3.290 |
| 262 | H | Gly | 329 | −7.104 | −2.853 | 2.403 |
| 263 | Cα | Gly | 329 | −7.017 | −3.981 | 4.152 |
| 264 | 1Hα | Gly | 329 | −7.829 | −4.470 | 4.690 |
| 265 | 2Hα | Gly | 329 | −6.531 | −4.746 | 3.545 |
| 266 | Qα | Gly | 329 | −7.180 | −4.608 | 4.118 |
| 267 | C | Gly | 329 | −6.014 | −3.400 | 5.149 |
| 268 | O | Gly | 329 | −5.932 | −2.184 | 5.315 |

Column Headings:
A = atom serial no. in peptide
B = atom type/position in amino acid
C = name of amino acid residue
D = residue number in peptide sequence
X, Y, Z = Orthogonal Coordinates (in Angstroms) for X, Y and Z axes

TABLE 6
Average HMR Coordinates of Self-Constrained R5A
Mimic Peptide-1 (“R5A-M1”)

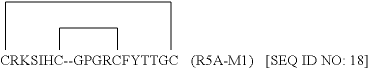

| A | B | C | D | X | Y | Z |
|---|---|---|---|---|---|---|
| 1 | Cα | Cys | 310 | 28.656 | −5.217 | −4.803 |
| 2 | Hα | Cys | 310 | 29.287 | −5.308 | −4.976 |
| 3 | Hβ1 | Cys | 310 | 28.482 | −5.354 | −4.846 |
| 4 | Hβ2 | Cys | 310 | 28.912 | −5.476 | −4.828 |
| 5 | C | Cys | 310 | 27.753 | −5.218 | −4.694 |
| 6 | O | Cys | 310 | 27.261 | −5.495 | −4.991 |
| 7 | Cβ | Cys | 310 | 28.875 | −5.381 | −4.974 |
| 8 | Sγ | Cys | 310 | 29.918 | −5.182 | −5.713 |
| 9 | N | Cys | 310 | 28.535 | −5.253 | −4.584 |
| 10 | HT1 | Cys | 310 | 28.587 | −5.033 | −4.335 |
| 11 | HT2 | Cys | 310 | 28.667 | −5.441 | −4.758 |
| 12 | HT3 | Cys | 310 | 28.207 | −5.254 | −4.600 |
| 13 | N | Arg | 311 | 27.549 | −4.855 | −−4.216 |
| 14 | HN | Arg | 311 | 27.970 | −4.649 | −3.983 |
| 15 | Cα | Arg | 311 | 26.696 | −4.756 | −4.028 |
| 16 | Hα | Arg | 311 | 26.248 | −5.036 | −4.341 |
| 17 | Cβ | Arg | 311 | 26.871 | −5.018 | −3.630 |
| 18 | Hβ1 | Arg | 311 | 26.963 | −5.418 | −3.810 |
| 19 | Hβ2 | Arg | 311 | 26.188 | −5.030 | −3.365 |
| 20 | Cγ | Arg | 311 | 27.839 | −4.686 | −3.322 |
| 21 | Hγ1 | Arg | 311 | 27.724 | −4.478 | −3.252 |
| 22 | Hγ2 | Arg | 311 | 28.235 | −4.698 | −3.389 |
| 23 | Cδ | Arg | 311 | 28.518 | −4.657 | −3.114 |
| 24 | Hδ1 | Arg | 311 | 28.796 | −4.608 | −2.814 |
| 25 | Hδ2 | Arg | 311 | 28.599 | −4.471 | −3.289 |
| 26 | Nε | Arg | 311 | 28.726 | −4.923 | −3.127 |
| 27 | Hε | Arg | 311 | 28.795 | −5.005 | −3.108 |
| 28 | CZ | Arg | 311 | 28.672 | −5.013 | −3.035 |
| 29 | NH1 | Arg | 311 | 28.514 | −4.950 | −3.061 |
| 30 | HH11 | Arg | 311 | 28.482 | −4.690 | −3.092 |
| 31 | HH12 | Arg | 311 | 28.501 | −4.981 | −2.998 |
| 32 | NH2 | Arg | 311 | 28.850 | −5.297 | −2.993 |
| 33 | HH21 | Arg | 311 | 28.950 | −5.351 | −3.067 |
| 34 | HH22 | Arg | 311 | 28.755 | −5.367 | −2.961 |
| 35 | C | Arg | 311 | 26.243 | −4.032 | −3.806 |
| 36 | O | Arg | 311 | 26.576 | −3.640 | −4.028 |
| 37 | N | Lys | 312 | 25.471 | −3.866 | −3.364 |
| 38 | HN | Lys | 312 | 25.238 | −4.201 | −3.211 |
| 39 | Cα | Lys | 312 | 24.975 | −3.186 | −3.121 |
| 40 | Hα | Lys | 312 | 25.313 | −2.820 | −3.392 |
| 41 | Cβ | Lys | 312 | 24.036 | −3.489 | −3.317 |
| 42 | Hβ1 | Lys | 312 | 23.630 | −3.170 | −3.080 |
| 43 | Hβ2 | Lys | 312 | 23.943 | −3.964 | −3.391 |
| 44 | Cγ | Lys | 312 | 23.822 | −3.517 | −3.758 |
| 45 | Hγ1 | Lys | 312 | 24.108 | −3.584 | −3.837 |
| 46 | Hγ2 | Lys | 312 | 23.655 | −3.364 | −3.598 |
| 47 | Cδ | Lys | 312 | 23.474 | −3.645 | −4.253 |
| 48 | Hδ1 | Lys | 312 | 23.363 | −3.427 | −4.362 |
| 49 | Hδ2 | Lys | 312 | 23.213 | −3.759 | −4.448 |
| 50 | Cε | Lys | 312 | 23.618 | −3.874 | −4.420 |
| 51 | Hε1 | Lys | 312 | 23.734 | −4.014 | 4.372 |
| 52 | Hε2 | Lys | 312 | 23.776 | −3.803 | −4.463 |
| 53 | NZ | Lys | 312 | 23.468 | −4.102 | −4.411 |
| 54 | HZ1 | Lys | 312 | 23.432 | −4.257 | −4.251 |
| 55 | HZ2 | Lys | 312 | 23.248 | −4.091 | −4.671 |
| 56 | HZ3 | Lys | 312 | 23.415 | −4.020 | −4.287 |
| 57 | C | Lys | 312 | 24.890 | −2.697 | −2.294 |
| 58 | O | Lys | 312 | 24.444 | −2.740 | −2.133 |
| 59 | N | Ser | 313 | 25.335 | −2.231 | −1.792 |
| 60 | HN | Ser | 313 | 25.676 | −2.221 | −1.954 |
| 61 | Cα | Ser | 313 | 25.308 | −1.729 | −0.986 |
| 62 | Hα | Ser | 313 | 24.642 | −1.598 | −0.953 |
| 63 | Cβ | Ser | 313 | 25.523 | −2.090 | −0.503 |
| 64 | Hβ1 | Ser | 313 | 25.976 | −1.849 | −0.297 |
| 65 | Hβ2 | Ser | 313 | 25.560 | −2.577 | −0.620 |
| 66 | Oγ | Ser | 313 | 25.102 | −2.094 | −0.183 |
| 67 | Hγ | Ser | 313 | 25.066 | −2.059 | −0.349 |
| 68 | C | Ser | 313 | 25.974 | −1.030 | −0.660 |
| 69 | O | Ser | 313 | 27.019 | −1.288 | −0.738 |
| 70 | N | Ile | 314 | 25.367 | −0.147 | −0.289 |
| 71 | HN | Ile | 314 | 24.537 | 0.013 | −0.243 |
| 72 | Cα | Ile | 314 | 25.898 | 0.584 | 0.046 |
| 73 | Hα | Ile | 314 | 26.853 | 0.315 | −0.113 |
| 74 | Cβ | Ile | 314 | 25.558 | 1.490 | −0.437 |
| 75 | Hβ | Ile | 314 | 25.539 | 1.779 | −0.079 |
| 76 | Cγ1 | Ile | 314 | 24.805 | 1.745 | −0.842 |
| 77 | Hγ11 | Ile | 314 | 24.493 | 1.668 | −0.837 |
| 78 | Hγ12 | Ile | 314 | 24.849 | 1.589 | −0.972 |
| 79 | Cγ2 | Ile | 314 | 26.056 | 1.736 | −1.031 |
| 80 | Hγ21 | Ile | 314 | 26.233 | 1.810 | −1.102 |
| 81 | Hγ22 | Ile | 314 | 26.050 | 1.578 | −1.164 |
| 82 | Hγ23 | Ile | 314 | 26.065 | 1.878 | −1.087 |
| 83 | Cδ1 | Ile | 314 | 24.355 | 2.304 | −1.129 |
| 84 | Hδ11 | Ile | 314 | 24.351 | 2.512 | −1.125 |
| 85 | Hδ12 | Ile | 314 | 24.243 | 2.491 | −1.018 |
| 86 | Hδ13 | Ile | 314 | 24.255 | 2.262 | −1.335 |
| 87 | C | Ile | 314 | 25.526 | 0.847 | 1.104 |
| 88 | O | Ile | 314 | 24.510 | 0.913 | 1.594 |
| 89 | N | His | 315 | 26.347 | 1.000 | 1.456 |
| 90 | HN | His | 315 | 27.142 | 0.939 | 1.030 |
| 91 | Cα | His | 315 | 26.104 | 1.255 | 2.456 |
| 92 | Hα | His | 315 | 25.179 | 1.360 | 2.815 |
| 93 | Cβ | His | 315 | 26.722 | 0.508 | 2.845 |
| 94 | Hβ1 | His | 315 | 27.220 | 0.688 | 3.083 |
| 95 | Hβ2 | His | 315 | 27.116 | 0.192 | 2.349 |
| 96 | Cγ | His | 315 | 26.162 | −0.037 | 3.590 |
| 97 | Nδ1 | His | 315 | 25.785 | −0.417 | 4.067 |
| 98 | Hδ1 | His | 315 | 25.840 | −0.283 | 4.124 |
| 99 | Cδ2 | His | 315 | 25.901 | −0.342 | 3.854 |
| 100 | Hδ2 | His | 315 | 26.074 | −0.203 | 3.576 |
| 101 | Cε1 | His | 315 | 25.352 | −0.788 | 4.726 |
| 102 | Hε1 | His | 315 | 24.954 | −1.220 | 5.190 |
| 103 | Nε2 | His | 315 | 25.356 | −0.912 | 4.523 |
| 104 | Hε2 | His | 315 | 25.104 | −1.150 | 4.866 |
| 105 | C | His | 315 | 26.481 | 2.111 | 2.624 |
| 106 | O | His | 315 | 27.345 | 1.998 | 2.253 |
| 107 | N | Cys | 316 | 25.827 | 2.973 | 3.189 |
| 108 | HN | Cys | 316 | 25.150 | 3.014 | 3.463 |
| 109 | Cα | Cys | 316 | 26.096 | 3.847 | 3.405 |
| 110 | Hα | Cys | 316 | 26.977 | 3.735 | 3.080 |
| 111 | Cβ | Cys | 316 | 25.517 | 4.621 | 2.991 |
| 112 | Hβ1 | Cys | 316 | 25.149 | 5.210 | 3.477 |
| 113 | Hβ2 | Cys | 316 | 24.990 | 4.410 | 2.736 |
| 114 | Sγ | Cys | 316 | 26.241 | 4.953 | 2.116 |
| 115 | Hγ | Cys | 316 | 25.833 | 4.949 | 1.952 |
| 116 | C | Cys | 316 | 25.770 | 4.148 | 4.479 |
| 117 | O | Cys | 316 | 24.944 | 4.246 | 4.982 |
| 118 | N | Gly | 319 | 26.415 | 4.297 | 4.824 |
| 119 | HN | Gly | 319 | 27.061 | 4.207 | 4.389 |
| 120 | Cα | Gly | 319 | 26.182 | 4.583 | 5.835 |
| 121 | Hα1 | Gly | 319 | 26.325 | 3.936 | 6.075 |
| 122 | Hα2 | Gly | 319 | 25.442 | 4.894 | 5.934 |
| 123 | C | Gly | 319 | 26.725 | 5.330 | 6.444 |
| 124 | O | Gly | 319 | 27.175 | 6.000 | 6.155 |
| 125 | N | Pro | 320 | 26.692 | 5.236 | 7.298 |
| 126 | Cα | Pro | 320 | 27.192 | 5.917 | 7.943 |
| 127 | Hα | Pro | 320 | 26.882 | 6.687 | 8.050 |
| 128 | Cβ | Pro | 320 | 26.914 | 5.566 | 8.788 |
| 129 | Hβ1 | Pro | 320 | 26.412 | 6.152 | 9.142 |
| 130 | Hβ2 | Pro | 320 | 27.562 | 5.400 | 9.098 |
| 131 | Cγ | Pro | 320 | 26.445 | 4.651 | 8.625 |
| 132 | Hγ1 | Pro | 320 | 25.748 | 4.633 | 9.043 |
| 133 | Hγ2 | Pro | 320 | 27.006 | 4.172 | 8.731 |
| 134 | Cδ | Pro | 320 | 26.175 | 4.460 | 7.700 |
| 135 | Hδ1 | Pro | 320 | 25.331 | 4.488 | 7.759 |
| 136 | Hδ2 | Pro | 320 | 26.488 | 3.780 | 7.306 |
| 137 | C | Pro | 320 | 28.373 | 5.830 | 7.683 |
| 138 | O | Pro | 320 | 28.662 | 5.648 | 7.907 |
| 139 | N | Gly | 321 | 29.012 | 5.967 | 7.206 |
| 140 | HN | Gly | 321 | 28.742 | 6.114 | 7.042 |
| 141 | Cα | Gly | 321 | 30.154 | 5.903 | 6.920 |
| 142 | Hα1 | Gly | 321 | 30.578 | 5.319 | 7.296 |
| 143 | Hα2 | Gly | 321 | 30.327 | 6.695 | 7.221 |
| 144 | C | Gly | 321 | 30.539 | 5.565 | 5.709 |
| 145 | O | Gly | 321 | 31.137 | 5.720 | 5.290 |
| 146 | N | Arg | 322 | 30.193 | 5.096 | 5.166 |
| 147 | HN | Arg | 322 | 29.717 | 4.992 | 5.542 |
| 148 | Cα | Arg | 322 | 30.509 | 4.734 | 3.991 |
| 149 | Hα | Arg | 322 | 30.590 | 5.187 | 3.737 |
| 150 | Cβ | Arg | 322 | 31.338 | 4.094 | 3.556 |
| 151 | Hβ1 | Arg | 322 | 31.730 | 3.964 | 3.092 |
| 152 | Hβ2 | Arg | 322 | 31.272 | 3.800 | 3.470 |
| 153 | Cγ | Arg | 322 | 31.663 | 4.027 | 3.868 |
| 154 | Hγ1 | Arg | 322 | 31.874 | 4.503 | 4.173 |
| 155 | Hγ2 | Arg | 322 | 31.971 | 3.991 | 3.713 |
| 156 | Cδ | Arg | 322 | 31.291 | 3.325 | 3.973 |
| 157 | Hδ1 | Arg | 322 | 30.911 | 2.959 | 3.659 |
| 158 | Hδ2 | Arg | 322 | 30.992 | 3.329 | 4.335 |
| 159 | Nε | Arg | 322 | 31.828 | 3.133 | 3.984 |
| 160 | Hε | Arg | 322 | 31.984 | 2.928 | 3.564 |
| 161 | CZ | Arg | 322 | 32.133 | 3.224 | 4.519 |
| 162 | NH1 | Arg | 322 | 31.936 | 3.506 | 5.094 |
| 163 | HH11 | Arg | 322 | 31.569 | 3.648 | 5.116 |
| 164 | HH12 | Arg | 322 | 32.170 | 3.570 | 5.498 |
| 165 | NH2 | Arg | 322 | 32.614 | 3.040 | 4.493 |
| 166 | HH21 | Arg | 322 | 32.762 | 2.828 | 4.060 |
| 167 | HH22 | Arg | 322 | 32.843 | 3.106 | 4.899 |
| 168 | C | Arg | 322 | 29.911 | 4.382 | 3.562 |
| 169 | O | Arg | 322 | 29.449 | 4.324 | 4.091 |
| 170 | N | Cys | 323 | 29.923 | 4.142 | 2.585 |
| 171 | HN | Cys | 323 | 30.308 | 4.203 | 2.207 |
| 172 | Cα | Cys | 323 | 29.383 | 3.792 | 2.079 |
| 173 | Hα | Cys | 323 | 28.784 | 3.625 | 2.617 |
| 174 | Cβ | Cys | 323 | 28.895 | 4.474 | 1.641 |
| 175 | Hβ1 | Cys | 323 | 29.056 | 4.537 | 1.325 |
| 176 | Hβ2 | Cys | 323 | 28.938 | 4.650 | 1.734 |
| 177 | Sγ | Cys | 323 | 28.026 | 4.903 | 1.536 |
| 178 | Hγ | Cys | 323 | 28.297 | 5.141 | 1.250 |
| 179 | C | Cys | 323 | 30.121 | 2.974 | 1.248 |
| 180 | O | Cys | 323 | 30.870 | 3.069 | 0.487 |
| 181 | N | Phe | 324 | 29.906 | 2.177 | 1.399 |
| 182 | HN | Phe | 324 | 29.299 | 2.143 | 2.022 |
| 183 | Cα | Phe | 324 | 30.542 | 1.336 | 0.664 |
| 184 | Hα | Phe | 324 | 31.179 | 1.386 | 0.101 |
| 185 | Cβ | Phe | 324 | 30.869 | 0.795 | 1.184 |
| 186 | Hβ1 | Phe | 324 | 31.080 | 1.131 | 1.020 |
| 187 | Hβ2 | Phe | 324 | 31.006 | 0.204 | 1.385 |
| 188 | Cγ | Phe | 324 | 30.773 | 0.594 | 1.672 |
| 189 | Cδ1 | Phe | 324 | 30.237 | 0.248 | 1.581 |
| 190 | Hδ1 | Phe | 324 | 29.885 | 0.147 | 1.136 |
| 191 | Cδ2 | Phe | 324 | 31.221 | 0.709 | 2.237 |
| 192 | Hδ2 | Phe | 324 | 31.644 | 0.970 | 2.310 |
| 193 | Cε1 | Phe | 324 | 30.140 | 0.050 | 2.016 |
| 194 | Hε1 | Phe | 324 | 29.717 | −0.211 | 1.941 |
| 195 | Cε2 | Phe | 324 | 31.126 | 0.512 | 2.674 |
| 196 | Hε2 | Phe | 324 | 31.479 | 0.613 | 3.117 |
| 197 | CZ | Phe | 324 | 30.589 | 0.178 | 2.583 |
| 198 | HZ | Phe | 324 | 30.514 | 0.023 | 2.928 |
| 199 | C | Phe | 324 | 30.059 | 0.958 | 0.138 |
| 200 | O | Phe | 324 | 28.994 | 1.011 | 0.169 |
| 201 | N | Tyr | 325 | 30.837 | 0.567 | −0.358 |
| 202 | HN | Tyr | 325 | 31.678 | 0.545 | −0.354 |
| 203 | Cα | Tyr | 325 | 30.490 | 0.179 | −0.891 |
| 204 | Hα | Tyr | 325 | 29.516 | 0.253 | −0.819 |
| 205 | Cβ | Tyr | 325 | 30.783 | 0.378 | −1.544 |
| 206 | Hβ1 | Tyr | 325 | 31.135 | 0.065 | −1.566 |
| 207 | Hβ2 | Tyr | 325 | 30.932 | 0.635 | −1.636 |
| 208 | Cγ | Tyr | 325 | 30.443 | 0.692 | −2.006 |
| 209 | Cδ1 | Tyr | 325 | 30.383 | 1.093 | −1.851 |
| 210 | Hδ1 | Tyr | 325 | 30.585 | 1.188 | −1.375 |
| 211 | Cδ2 | Tyr | 325 | 30.181 | 0.584 | −2.613 |
| 212 | Hδ2 | Tyr | 325 | 30.226 | 0.277 | −2.738 |
| 213 | Cε1 | Tyr | 325 | 30.070 | 1.396 | −2.268 |
| 214 | Hε1 | Tyr | 325 | 30.026 | 1.703 | −2.141 |
| 215 | Cε2 | Tyr | 325 | 29.868 | 0.885 | −3.032 |
| 216 | Hε2 | Tyr | 325 | 29.665 | 0.789 | −3.507 |
| 217 | CZ | Tyr | 325 | 29.817 | 1.277 | −2.871 |
| 218 | OH | Tyr | 325 | 29.508 | 1.570 | −3.289 |
| 219 | HH | Tyr | 325 | 29.457 | 1.335 | −3.209 |
| 220 | C | Tyr | 325 | 31.113 | −0.550 | −0.912 |
| 221 | O | Tyr | 325 | 32.225 | −0.733 | −0.875 |
| 222 | N | Thr | 326 | 30.413 | −0.967 | −0.976 |
| 223 | HN | Thr | 326 | 29.531 | −0.790 | −1.004 |
| 224 | Cα | Thr | 326 | 30.894 | −1.679 | −1.005 |
| 225 | Hα | Thr | 326 | 31.891 | −1.765 | −0.952 |
| 226 | Cβ | Thr | 326 | 30.223 | −1.982 | −0.482 |
| 227 | Hβ | Thr | 326 | 30.084 | −2.172 | −0.262 |
| 228 | Oγ1 | Thr | 326 | 29.681 | −2.120 | −0.153 |
| 229 | Hγ1 | Thr | 326 | 29.263 | −1.941 | −0.026 |
| 230 | Cγ2 | Thr | 326 | 30.322 | −1.949 | −0.367 |
| 231 | Hγ21 | Thr | 326 | 30.358 | −1.716 | −0.503 |
| 232 | Hγ22 | Thr | 326 | 30.535 | −2.067 | −0.544 |
| 233 | Hγ23 | Thr | 326 | 30.188 | −2.063 | −0.052 |
| 234 | C | Thr | 326 | 30.742 | −1.968 | −1.649 |
| 235 | O | Thr | 326 | 29.811 | −2.011 | −1.913 |
| 236 | N | Thr | 327 | 31.653 | −2.177 | −1.912 |
| 237 | HN | Thr | 327 | 32.370 | −2.128 | −1.676 |
| 238 | Cα | Thr | 327 | 31.621 | −2.467 | −2.525 |
| 239 | Hα | Thr | 327 | 30.678 | −2.525 | −2.633 |
| 240 | Cβ | Thr | 327 | 32.133 | −2.101 | −3.046 |
| 241 | Hβ | Thr | 327 | 32.393 | −2.418 | −3.305 |
| 242 | Oγ1 | Thr | 327 | 33.035 | −1.863 | −2.840 |
| 243 | Hγ1 | Thr | 327 | 33.259 | −1.764 | −2.995 |
| 244 | Cγ2 | Thr | 327 | 31.393 | −1.563 | −3.455 |
| 245 | Hγ21 | Thr | 327 | 31.592 | −1.478 | −3.800 |
| 246 | Hγ22 | Thr | 327 | 31.186 | −1.249 | −3.294 |
| 247 | Hγ23 | Thr | 327 | 30.878 | −1.588 | −3.533 |
| 248 | C | Thr | 327 | 32.313 | −3.060 | −2.514 |
| 249 | O | Thr | 327 | 33.292 | −3.130 | −2.446 |
| 250 | N | Gly | 328 | 31.830 | −3.490 | −2.581 |
| 251 | HN | Gly | 328 | 31.050 | −3.406 | −2.634 |
| 252 | Cα | Gly | 328 | 32.413 | −4.069 | −2.573 |
| 253 | Hα1 | Gly | 328 | 32.281 | −4.276 | −2.088 |
| 254 | Hα2 | Gly | 328 | 33.228 | −3.760 | −2.670 |
| 255 | C | Gly | 328 | 32.141 | −4.793 | −3.081 |
| 256 | O | Gly | 328 | 31.537 | −5.412 | −2.865 |
| 257 | N | Cys | 329 | 32.574 | −4.725 | −3.754 |
| 258 | HN | Cys | 329 | 33.050 | −4.215 | −3.892 |
| 259 | Cα | Cys | 329 | 32.370 | −5.385 | −4.293 |
| 260 | Hα | Cys | 329 | 31.705 | −5.808 | −4.056 |
| 261 | Hβ1 | Cys | 329 | 32.255 | −4.977 | −5.333 |
| 262 | Hβ2 | Cys | 329 | 31.585 | −4.831 | −4.940 |
| 263 | Cβ | Cys | 329 | 31.880 | −5.107 | −5.043 |
| 264 | Sγ | Cys | 329 | 31.143 | −5.418 | −5.662 |
| 265 | C | Cys | 329 | 33.548 | −5.774 | −4.421 |
| 266 | OT1 | Cys | 329 | 33.922 | −6.290 | −4.413 |
| 267 | OT2 | Cys | 329 | 34.074 | −5.551 | −4.524 |

Column Headings:
A = atom serial no. in peptide
B = atom type/position in amino acid
C = name of amino acid residue
D = residue number in peptide sequence
X, Y, Z = Orthogonal Coordinates (in Angstroms) for X, Y and Z axes

TABLE 7
Protein Database Search Results

| | Patterm | | | |
|---|---|---|---|---|
| —S_ali | mismatch | RMSD | N | Definition |
| 1bko_a.ali | 0 | 0.701713 | 1 | thymidylate synthase a |
| 1kh2_a.ali | 0 | 0.724791 | 2 | Argininosuccinate synthetase |
| 1kh2_c.ali | 0 | 0.736369 | 3 | Argininosuccinate synthetase |
| 1kh1_a.ali | 0 | 0.737859 | 4 | Argininosuccinate synthetase |
| 1bko_c.ali | 0 | 0.746757 | 5 | thymidylate synthase a |
| 1kor_c.ali | 0 | 0.752247 | 6 | Argininosuccinate synthetase |
| 1kor_a.ali | 0 | 0.754154 | 7 | Argininosuccinate synthetase |
| 1cc1_l.ali | 0 | 0.759915 | 8 | hydrogenase (small subunit) |
| 1bkp_a.ali | 0 | 0.772811 | 9 | thymidylate synthase a |
| 1aor_a.ali | 0 | 0.800599 | 10 | aldehyde ferredoxin oxidoreductase prot. cplxd with molybdopterin |
| 1ko6_a.ali | 0 | 0.811169 | 11 | nuclear pore complex protein nup98 |
| 1f32_a.ali | 0 | 0.815431 | 12 | major pepsin inhibitor pi-3 |
| 2fok_b.ali | 0 | 0.833307 | 13 | foki restriction endonuclease |
| 1h0m_c.ali | 0 | 0.833952 | 14 | transcriptional activator protein trar |
| 1ig0_a.ali | 0 | 0.928751 | 15 | thiamin pyrophosphokinase |
| 1j9o_a.ali | 0 | 0.997041 | 16 | Lymphotactin |
| 1rto_a.ali | 0 | 1.039156 | 17 | RANTES (nmr, min avg structure) |
| 1dky_a.ali | 0 | 1.048116 | 18 | Dnak |
| 1af2_a.ali | 0 | 1.05136 | 19 | cytidine deaminase |
| 1j8i_a.ali | 0 | 1.059596 | 20 | lymphotactin |
| 1ctt_m.ali | 0 | 1.063211 | 21 | cytidine deaminase (e.c.3.5.4.5) cplx with 3,4-dihydrozebularine |
| 1aln_m.ali | 0 | 1.069355 | 22 | cytidine deaminase |
| 1rtn_a.ali | 0 | 1.071115 | 23 | RANTES, nmr, 20 structures |
| 1mt3_a.ali | 0 | 1.092868 | 24 | Proline iminopeptidase |
| 1cer_o.ali | 0 | 1.130544 | 25 | holo-d-glyceraldehyde-3-phosphate dehydrogenase |
| 1lb1_g.ali | 0 | 1.138062 | 26 | guanine nucleotide exchange factor dbs |
| 1l0o_b.ali | 0 | 1.138922 | 27 | anti-sigma f factor |
| 1lb1_a.ali | 0 | 1.139799 | 28 | guanine nucleotide exchange factor dbs |
| 1b53_a.ali | 0 | 1.140268 | 29 | MIP-1α |
| 1lb1_c.ali | 0 | 1.146098 | 30 | Guanine nucleotide exchange factor dbs |
| 1lb1_e.ali | 0 | 1.151823 | 31 | Guanine nucleotide exchange factor dbs |
| 1hfg_a.ali | 0 | 1.188755 | 32 | viral macrophage inflamm. protein-ii (syn: vmip-ii, vmip-1b) |
| 1l3l_c.ali | 0 | 1.189487 | 33 | transcriptional activator protein trar |
| 1g91_a.ali | 0 | 1.194075 | 34 | myeloid progenitor inhibitory factor-1 |
| 1h0m_a.ali | 0 | 1.219714 | 35 | transcriptional activator protein trar |
| 1l3;_a.ali | 0 | 1.233692 | 36 | transcriptional activator protein trar |
| 1g9a_a.ali | 0 | 1.237861 | 37 | botulinum neurotoxin type b |
| 1epw_a.ali | 0 | 1.253715 | 38 | botulinum neurotoxin type b |
| 1ile_a.ali | 0 | 1.26135 | 39 | botulinum neurotoxin type b |
| 1hyu_a.ali | 0 | 1.264085 | 40 | alkyl hydroperoxide reductase subunit f |
| 1cja_a.ali | 0 | 1.301841 | 41 | actin-fragmin kinase |
| 1k5y_j.ali | 0 | 1.34896 | 42 | 5.8 s/25 s ribosomal RNA |
| 1hrj_a.ali | 0 | 1.397013 | 43 | human RANTES |
| 1cer_q.ali | 0 | 1.418202 | 44 | holo-d-glyceraldehyde-3-phosphate dehydrogenase |
| 1kh1_c.ali | 0 | 1.498184 | 45 | argininosuccinate synthetase |

TABLE 8
α Defensin pattern search

| | Pattern | | | |
|---|---|---|---|---|
| —S_ali | Mismatch | RMSD | N | Definition |
| 1gpi_a.ali | 0 | 0.761068 | 1 | “exoglucanase i” |
| 1e9y_b.ali | 0 | 1.02693 | 2 | “urease alpha subunit” |
| 1e9z_b.ali | 0 | 1.128919 | 3 | “urease alpha subunit” |
| 1flf_a.ali | 0 | 1.17168 | 4 | “pap-specific phosphatase” |
| 1j8c_a.ali | 0 | 1.219165 | 5 | “ubiquitin-like protein |
| | | | | hplic-2” |
| 1dfn_a.ali | 0 | 1.37678 | 6 | “defensin/hnp-3” |

TABLE 9
IMMUNIZATION PROTOCOL WITH V3/α-DEFENSIN

| | | |
|---|---|---|
| Priming (x3) | Boosting (x2) | Expected Result* |

CONTROL GROUPS

| | | |
|---|---|---|
| empty DNA vector | V3-FP_{JR-CSF} | ± |
| empty DNA vector | Chimeric V3/α-defensin-tt | ± |
| empty DNA vector + tt | Chimeric V3/α-defensin-tt | ± |
| empty DNA vector | Gp120_{JR-FL} | ± |
| empty DNA vector | Adjuvant and tt | −− |

EXPERIMENTAL GROUPS:

| | | |
|---|---|---|
| gp120 DNA vaccine | V3-FP_{JR-CSF} | +++ |
| gp120 DNA vaccine | chimeric V3/α-defensin-tt | +++ |
| gp120 DNA vaccine + tt | chimeric V3/α-defensin-tt | +++++ |
| gp120 DNA vaccine | gp120_{JR-FL} | + |
| gp120 DNA vaccine | adjuvant and tt | −− |

*relative level or titer of broadly neutralizing anti-V3 antibodies

TABLE 10
IMMUNIZATION WITH V3/BBI

| | | |
|---|---|---|
| Priming (x3) | Boosting (x2) | Expected Result* |

CONTROL GROUPS:

| | | |
|---|---|---|
| empty DNA vector | V3-FP_{JR-CSF} | ± |
| empty DNA vector | chimeric V3/BBI-tt | ± |
| empty DNA vector + tt | chimeric V3/BBI-tt | ± |
| empty DNA vector | gp120_{JR-FL} | ± |
| empty DNA vector | adjuvant and tt | −− |

EXPERIMENTAL GROUPS:

| | | |
|---|---|---|
| gp120 DNA vector | V3-FP_{JR-CSF} | +++ |
| gp120 DNA vector | chimeric V3/BBI-tt | +++ |
| gp120 DNA vector + tt | chimeric V3/BBI-tt | +++++ |
| gp120 DNA vector | gp120_{JR-FL} | + |
| gp120 DNA vector | adjuvant and tt | −− |

*relative level or titer of broadly neutralizing anti-V3 antibodies

1-55. (canceled)

56. A composition comprising an isolated peptide molecule or an isostere or a non-peptidic molecular mimetic thereof, which peptide, isostere, or mimetic mimics the three dimensional (3D) atomic structural conformation of a V3 loop peptide of HIV-1 envelope glycoprotein gp120 that is bound to, and constrained by, human monoclonal antibody (mAb) 447-52D, murine mAb 0.5β or an antigen binding fragment of the mAb, wherein the constrained V3 loop peptide differs in conformation from the same V3 loop peptide when it is in free form.

57. The composition of claim 56 wherein the conformation of the peptide, isostere, or mimetic mimics the 3D atomic structural conformation of the V3 loop peptide bound to and constrained by said mAb 447-52D or its antigen binding fragment.

58. The composition of claim 57, wherein:
the 3D conformation is a solution structure determined by nuclear magnetic resonance (NMR) spectroscopy, and
said conformation is defined by a set of NMR structure coordinates having a root mean square deviation (rmsd) of not more than about 2 Å in the backbone atoms from the sets of structure coordinates in Table 3 or Table 4.

59. The composition of claim 58 wherein the conformation is defined by a set of NMR structure coordinates having a rmsd of not more than about 1.8 Å in the backbone atoms from the sets of structure coordinates in Table 3 or Table 4.

60. The composition of claim 59 wherein the conformation is defined by a set of NMR structure coordinates having a rmsd of not more than about 1.5 Å in the backbone atoms from the sets of structure coordinates in Table 3 or Table 4.

61. The composition of claim 56 wherein the V3 loop peptide has the amino acid sequence of a segment within the V3 loop of the gp120 protein of HIV-1_{MN} or HIV-1_{IIIB}.

62. The composition of claim 56 wherein the isolated peptide has an amino acid sequence that is
(a) a substitution variant of no more than 6 substituted amino acid residues of KSIHIGPGRAFYTT (SEQ ID NO:17), KRIHIGPGRAFYTT (SEQ ID NO:20) or KSIRIQRGPGRAFVTIG (SEQ ID NO:28), or, further, when said isolated peptide is a cyclic peptide, up two or four additional substitutions or additions of Cys residues; or
(b) a terminal or internal addition variant of no more than about 20 added amino acid residues of KSIHIGPGRAFYTT (SEQ ID NO:17), KRIHIGPGRAFYTT (SEQ ID NO:20), KSIRIQRGPGRAFVTIG (SEQ ID NO:28), or of said substitution variant of (a); or
(c) a deletion variant of no more than 3 deleted residues at one or both termini of KSIHIGPGRAFYTT (SEQ ID NO:17), KRIHIGPGRAFYTT (SEQ ID NO:20) or KSIRIQRGPGRAFVTIG (SEQ ID. NO:28).

63. The composition of claim 58 wherein the isolated peptide has an amino acid sequence that is
(a) a substitution variant of no more than 6 substituted amino acid residues of KSIHIGPGRAFYTT (SEQ ID NO:17), KRIHIGPGRAFYTT (SEQ ID NO:20) or KSIRIQRGPGRAFVTIG (SEQ ID NO:28) or, further, when said isolated peptide is a cyclic peptide, up two or four additional substitutions or additions of Cys residues; or
(b) a terminal or internal addition variant of no more than about 20 added amino acid residues of KSIHIGPGRAFYTT (SEQ ID NO:17), KRIHIGPGRAFYTT (SEQ ID NO:20), KSIRIQRGPGRAFVTIG (SEQ ID NO:28), or of said substitution variant of (a); or
(c) a deletion variant of no more than 3 deleted residues at one or both termini of KSIHIGPGRAFYTT (SEQ ID NO:17), KRIHIGPGRAFYTT (SEQ ID NO:20) or KSIRIQRGPGRAFVTIG (SEQ ID NO:28).

64. The composition of claim 56 wherein the isolated peptide is a cyclic peptide.

65. The composition of claim 58 wherein the isolated peptide is a cyclic peptide.

66. The composition of claim 62 wherein the isolated peptide is a cyclic peptide.

67. The composition of claim 64 wherein the cyclic peptide is constrained by one or two internal disulfide bridges.

68. The composition of claim 66 wherein the cyclic peptide is constrained by one or two internal disulfide bridges.

69. The composition of claim 67 wherein the isolated peptide is any one of:

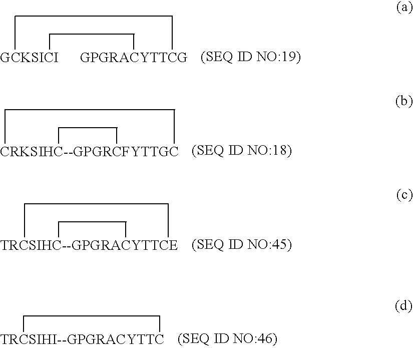

70. The composition of claim 58 wherein the isolated peptide binds selectively to CCR5 (R5) chemokine receptors.

71. The composition of claim 62 wherein the isolated peptide binds selectively to R5 chemokine receptors.

72. The composition of claim 67 wherein the isolated peptide binds selectively to R5 chemokine receptors.

73. The composition of claim 68 wherein the isolated peptide binds selectively to R5 chemokine receptors.

74. The composition of claim 58 wherein the isolated peptide binds selectively to CXCR4 (X4) chemokine receptors.

75. The composition of claim 62 wherein the isolated peptide binds selectively to X4 chemokine receptors.

76. The composition of claim 67 wherein the isolated peptide binds selectively to X4 chemokine receptors.

77. The composition of claim 68 wherein the isolated peptide binds selectively to X4 chemokine receptors.

78. The composition of claim 57 wherein the isolated peptide binds to mAb 447-52D or an antigen binding fragment thereof with an affinity characterized by a dissociation constant (K_{d}) of about 100 nM or lower.

79. An antibody-complexed composition comprising a complex between
(a) human mAb 447-52D or an antigen binding fragment thereof and
(b) the peptide, isostere or mimetic according to claim 57.

80. The antibody-complexed composition of claim 80 wherein the 3D structure is a solution structure determined by NMR spectroscopy and the peptide, isostere or mimetic is defined by a set of NMR structure coordinates having a rmsd of not more than about 2 Å in the backbone atoms from the set of structure coordinates in Table 3 or Table 4.

81. The antibody-complexed composition of claim 80 wherein the peptide has an amino acid sequence that is
(a) a substitution variant of no more than 6 substituted amino acid residues of KRIHIGPGRAFYTT (SEQ ID NO:20) or KSIRIQRGPGRAFVTIG (SEQ ID NO:28) or, further, when said isolated peptide is a cyclic peptide, up two or four additional substitutions or additions of Cys residues; or
(b) an addition variant of no more than about 20 added amino acid residues of KRIHIGPGRAFYTT (SEQ ID NO:20) or KSIRIQRGPGRAFVTIG (SEQ ID NO:28); or of said substitution variant of (a); or
(c) a deletion variant of no more than 3 deleted residues at one or both termini of KRIHIGPGRAFYTT (SEQ ID NO:20) or KSIRIQRGPGRAFVTIG (SEQ ID NO:28).

82. A method of identifying from among a plurality of existing compounds a molecule that is useful as an HIV-1 V3 loop immunogen or as an inhibitor of binding of HIV-1 to a chemokine receptor/HIV-1 co-receptor on the surface of a receptor-bearing target cell, which method comprises:
(a) either or both of:
(i) subjecting to NMR analysis and solving the structure of a peptide that is specifically bound to mAb 447-52D and/or mAb 0.5β, or to an antigen binding fragment of said mAb with an affinity characterized by a K_{d} of up to 1 μM; and
(ii) employing the NMR coordinates listed in any of Tables 3-6; and
(b) screening said plurality of compounds for a compound that has the following characteristics:
(i) a conformation defined by NMR structure coordinates having a rmsd of not more than about 2 Å in the backbone atoms from NMR coordinates determined in step (a)(i) or the NMR coordinates listed in any of Tables 3-6;
(ii) specifically binds to mAb 447-52D or 0.5β with an affinity characterized by a K_{d} of up to about 100 nM;
(iii) selectively binds to either or both of the chemokine receptors R5 and X4 with an affinity characterized by a K_{d} of up to 1 μM as measured in a receptor binding assay;
which screening steps and characteristic determination are performed by either computational means, experimental means, or both, and
said molecule is identified as being a useful immunogen or inhibitor if it has the characteristics of (b)(i), (ii) and (iii), and
(1) if the molecule is a natural peptide comprising L-amino acids, the molecule does not have a sequence that is a native, uninterrupted sequence of
(A) an HIV V3 loop peptide epitope, or
(B) a chemokine selected from MIP-1α, MIP-1β, RANTES, SDF-1, or a fragment of said chemokine, that binds to said receptor,
or
(2) the molecule is a partial peptide or non-peptidic peptidomimetic compound.

83. The method of claim 82 wherein the screening step (b) screens for selective binding to R5 chemokine receptors.

84. The method of claim 82 wherein the screening step (b) screens for selective binding to X4 chemokine receptors.

85. The method of claim 82 wherein the screening step (b) screens for specific binding to said mAbs with an affinity characterized by a K_{d} of about 10 nM or lower.

86. A method for designing a molecule that is a peptide, isostere or mimetic according to claim 56, and that is useful as an HIV-1 V3 loop immunogen or as an inhibitor of binding of HIV-1 to a chemokine receptor/HIV-1 co-receptor on the surface of a receptor-bearing target cell, which method comprises:
(a) either or both of:
(i) subjecting to NMR analysis and solving the structure of a peptide that is specifically bound by mAb 447-52D and/or mAb 0.5β or an antigen binding fragment of said mAb with an affinity characterized by a K_{d} of up to about 1 μM; and
(ii) employing the NMR coordinates listed in any of Tables 3-6; and
(b) determining conformational parameters of the molecule being designed such that the molecule has a β-hairpin structure and a hydrogen bonding network that result in a conformation defined by NMR structure coordinates having a rmsd of not more than about 2 Å in the backbone atoms from NMR coordinates determined in step (a)(i) or listed in any one of Tables 3-6; and
(c) if the molecule being designed is a peptide or partial peptide, choosing amino acids and a sequence that will result in a molecule with the conformational parameters set out in (b),
thereby designing said molecule.

87. A method for making a molecule that is useful as an HIV-1 V3 loop immunogen or as an inhibitor of binding of HIV-1 to a chemokine receptor/HIV-1 co-receptor on the surface of a receptor-bearing target cell, which method comprises:
(a) designing the molecule in accordance with the method of claim 86;
(b) selecting a synthetic process that will stabilize said β-hairpin structure and hydrogen bonding network; and
(c) employing the synthetic process of (b) to synthesize the molecule.

88. The method of claim 87, further comprising:
(d) testing the molecule produced in step (c) for one or both of:
(i) specific binding to an anti-HIV-1 V3-specific neutralizing mAb or antigen binding fragment thereof, and
(ii) selective binding to either or both R5 and X4 chemokine receptors.

89. The method of claim 88 wherein neutralizing mAb of step (d)(i) is human mAb 447-52D or murine mAb 0.5β.

90. The method of claim 88 further comprising selecting as useful a molecule with following characteristics:
(i) specifically binds to said neutralizing mAb or fragment with an affinity characterized by a K_{d} of up to about 100 nM; and
(ii) selectively binds to said chemokine receptor with an affinity characterized by a K_{d} of up to 1 μM as measured in a receptor binding assay.

91. The method of claim 87, wherein said β-hairpin structure is stabilized by internal disulfide linkages between Cys residues, internal hydrazone linkages or backbone cyclization using disubstituted amino acids.

92. The method of claim 87, further comprising the step of testing the molecule for one or more of the following activities:
(a) inhibition of binding of gp120 or said V3 loop peptide to said mAb or fragment;
(b) inhibition of binding to said chemokine receptor of a chemokine selected from MIP-1α, MIP-1β, RANTES, SDF-1, or a fragment of said chemokine;
(c) inhibition of gp120 or HIV-1 binding or attachment to said chemokine receptors/HIV co-receptors or to target cells expressing said receptors/co-receptors; and
(d) inhibition of HIV-1 infectivity of cells in culture; and
selecting as useful a molecule that scores positive for one of more of said inhibitory activities (a)-(d).

93. A composition that is useful as an HIV-1 V3 loop immunogen or as an inhibitor of binding of HIV-1 to a chemokine receptor/HIV-1 co-receptor on the surface of a receptor-bearing target cell, comprising a molecule designed in accordance with claim 86.

94. An immunogenic composition for induction of an anti-HIV-1 antibody response specific for a V3 loop epitope, comprising
(a) the composition of claim 56; and
(b) an immunologically acceptable excipient.

95. The immunogenic composition of claim 94 wherein the isolated peptide molecule, isostere or non-peptidic molecular mimetic is fused or conjugated to an immunogenic carrier.

96. The immunogenic composition of claim 94 that further comprises an adjuvant.

97. A pharmaceutical composition useful for blocking the interaction of HIV-1 with an R5 or X4 co-receptor and thereby inhibiting HIV-1 infectivity, comprising
(a) the composition of claim 56; and
(b) a pharmaceutically acceptable carrier or excipient.

98. A method for inducing in a subject an anti-HIV-1 neutralizing antibody response specific for a V3 loop epitope, comprising administering to the subject the immunogenic composition of claim 94.

99. A method for inducing in a subject an anti-HIV-1 neutralizing antibody response specific for a V3 loop epitope, comprising administering to the subject an immunogenic composition comprising the composition of claim 59 and an immunologically acceptable excipient.

100. A method for inducing in a subject an anti-HIV-1 neutralizing antibody response specific for a V3 loop epitope, comprising administering to the subject an immunogenic composition comprising the composition of claim 62 and an immunologically acceptable excipient.

101. A method for inducing in a subject an anti-HIV-1 neutralizing antibody response specific for a V3 loop epitope, comprising administering to the subject an immunogenic composition comprising the composition of claim 67 and an immunologically acceptable excipient.

102. A method for inducing in a subject an anti-HIV-1 neutralizing antibody response specific for a V3 loop epitope, comprising administering to the subject an immunogenic composition comprising the composition of claim 62 and an immunologically acceptable excipient.

103. The method of claim 98 wherein said subject if infected with, or at risk of infection with, HIV-1.

104. A method of inhibiting infection by HIV-1, comprising providing to cells at risk for said infection and infection-inhibiting effective amount of the composition of claim 56.

105. A method of inhibiting infection by HIV-1, comprising providing to cells at risk for said infection and infection-inhibiting effective amount of the composition of claim 58.

106. A method of inhibiting infection by HIV-1, comprising providing to cells at risk for said infection and infection-inhibiting effective amount of the composition of claim 62.

107. The method of claim 137 wherein said providing and inhibiting is in vivo.

108. A method of preventing an HIV-1 infection in an uninfected subject at risk for such infection or for inhibiting viral spread and disease progression in an infected subject, comprising administering to a subject in need of said prevention or inhibition an effective amount of the pharmaceutical composition of claim 97.

109. A method of preventing an HIV-1 infection in an uninfected subject at risk for such infection or for inhibiting viral spread and disease progression in an infected subject, comprising administering to a subject in need of said prevention or inhibition an effective amount of a pharmaceutical composition comprising the composition of claim 62 and a pharmaceutically acceptable carrier or excipient.

110. A method of preventing an HIV-1 infection in an uninfected subject at risk for such infection or for inhibiting viral spread and disease progression in an infected subject, comprising administering to a subject in need of said prevention or inhibition an effective amount of a pharmaceutical composition comprising the composition of claim 69 and a pharmaceutically acceptable carrier or excipient.

111. A computing platform for generating a 3D model of a conformationally constrained HIV V3 loop peptide as described in claim 56, which computing platform comprises:
(a) a data storage device storing data comprising a set of structural coordinates defining the structure of the constrained V3 loop peptide, and
(b) a data processing unit for generating the 3D model from the data stored in said data-storage device.

112. A computer generated model representing the conformationally constrained structure of a V3 loop peptide as described in claim 56, the computer generated model having a 3D atomic structure defined by a set of NMR coordinates set out in any of Tables 3-6.

113. A computer readable medium comprising, in a retrievable format, data that includes a set of structure coordinates defining a 3D structure of a conformationally constrained V3 loop peptide as described in claim 56.

114. The computer readable medium of claim 113, wherein said structure coordinates defining a said 3D structure correspond to a set of NMR coordinates which have a rmsd of not more than about 2 Å in the backbone atoms from the sets of structure coordinates in any of Tables 3-6.
